SEQUENCE LISTING (141 sequences)

SEQ ID NO: 1 - DNA, 1314 bp, Mycobacterium smegmatis
atgaccacattcaaagaacgcgagacgtccacagcggaccgcaagctcaccctggccgagatcctcgagatcttcgccgcgggtaaggagccgctgaagttcactgcgtacgacggcagctcggccggtcccgaggacgccacgatgggtctggacctcaagaccccgcgtgggaccacctatctggccacggcacccggcgatctgggcctggcccgtgcgtatgtctccggtgacctggagccgcacggcgtgcatcccggcgatccctacccgctgctgcgcgccctggccgaacgcatggagttcaagcgcccgcctgcgcgtgtgctggcgaacatcgtgcgctccatcggcatcgagcacctcaagccgatcgcaccgccgccgcaggaggcgctgccccggtggcgccgcatcatggagggcctgcggcacagcaagacccgcgacgccgaggccatccaccaccactacgacgtgtcgaacacgttctacgagtgggtgctgggcccgtcgatgacctacacgtgcgcgtgctaccccaccgaggacgcgaccctcgaagaggcccaggacaacaagtaccgcctggtgttcgagaagctgcgcctgaagcccggtgaccggttgctcgacgtgggctgcggctggggcggcatggtccgctacgcggcccgccacggcgtcaaggcgctcggtgtcacgctcagccgcgaacaggcgacgtgggcgcagaaggccatcgcccaggaaggtctcaccgatctggccgaggtgcgtcacggtgattaccgcgacgtcatcgaatccgggttcgacgcggtgtcctcgatcgggctgaccgagcacatcggcgtgcacaactacccggcgtacttcaacttcctcaagtcgaagctgcgcaccggtggcctgctgctcaaccactgcatcacccgcccggacaaccggtcggcgccatcggccggcgggttcatcgacaggtacgtgttccccgacggggagctcaccggctcgggccgcatcatcaccgaggcccaggacgtgggccttgaggtgatccacgaggagaacctacgcaatcactatgcgatgacgctgcgcgactggtgccgcaacctggtcgagcactgggacgaggcggtcgaagaggtcgggctgcccaccgcgaaggtgtggggcctgtacatggccggctcacgtctgggcttcgagaccaatgtggttcagctgcaccaggttctggcggtcaagcttgacgatcagggcaaggacggcggactgccgttgcggccctggtggtccgcctag

SEQ ID NO: 2 - protein, 437 aa, Mycobacterium smegmatis
MTTFKERETSTADRKLTLAEILEIFAAGKEPLKFTAYDGSSAGPEDATMGLDLKTPRGTTYLATAPGDLGLARAYVSGDLEPHGVHPGDPYPLLRALAERMEFKRPPARVLANIVRSIGIEHLKPIAPPPQEALPRWRRIMEGLRHSKTRDAEAIHHHYDVSNTFYEWVLGPSMTYTCACYPTEDATLEEAQDNKYRLVFEKLRLKPGDRLLDVGCGWGGMVRYAARHGVKALGVTLSREQATWAQKAIAQEGLTDLAEVRHGDYRDVIESGFDAVSSIGLTEHIGVHNYPAYFNFLKSKLRTGGLLLNHCITRPDNRSAPSAGGFIDRYVFPDGELTGSGRIITEAQDVGLEVIHEENLRNHYAMTLRDWCRNLVEHWDEAVEEVGLPTAKVWGLYMAGSRLGFETNVVQLHQVLAVKLDDQGKDGGLPLRPWWSA

SEQ ID NO: 3 - DNA, 1254 bp, Agromyces subbeticus
atcctcgagatcgtcgtcgccggtcggctgccgctgaggttcaccgcctacgacgggagctcggcggggccgcctgacgccctgttcggcctcgacctgaagactccgcgaggaacgacctatctcgccaccggccgcggcgatctcggcctcgcccgcgcctacatcgcgggcgacctcgagatacagggggtgcaccccggagacccctacgagctgctcaaggcactcgccgacagcctggtcttcaagctgccaccgccgcgggtgatgacccagatcatccgttcgatcggcgtcgaacatctgcggccgatcgcgccgccgccgcaagaggtgccgccccggtggcgccgcatcgccgaggggctccgacacagcaagggccgcgacgccgaagcgatccaccaccactacgacgtgtcgaacaccttctacgaatgggtgctcgggccgtcgatgacctacacgtgcgcgtgctacccgggcctcgacgcatccctcgacgaggcgcagcagaacaagtaccggctcgtgttcgagaagctgcggctgaagccgggcgaccgactgctcgacgtcggctgcgggtggggcggcatggtgcgctacgccgcgcgccacggcgtgcaggcgttgggcgtgaccctgtcgcgagagcagacggcgtgggcgcagcaggcgatcgccgtcgagggcctcgccgacctcgccgaggtgcgctacggcgactaccgcgacatcgccgaagacggcttcgatgcggtgtcatcgatcgggctgctcgagcacatcggcgtgcgcaactacgcttcgtatttcggctttctgcagtcgcgcttgcggcccgggggactcttgctcaaccactgcatcacccggcccgacaatcgctccgagccgtcggcgcgcggcttcatcgaccggtacgtgttccccgacggagagctcaccggctcgggccgcatcatcaccgaggcgcaggatgtcggcttcgaagtgctgcacgaagagaacctgcgtcagcattatgcactgacactgcgcgattggtgcgccaacctcgtcgcgcactgggaagaggcggtcgccgaggtcgggctgccgaccgcgaaggtgtggggcctctacatggccgggtcacggctcgcgttcgagagcggcggcatccagttgcaccaggtgctggcggtcagaccagacgatcgcagcgacgccgcccagctgccgctgcggccgtggtggacgccatag

SEQ ID NO: 4 - protein, 417 aa, Agromyces subbeticus
ILEIVVAGRLPLRFTAYDGSSAGPPDALFGLDLKTPRGTTYLATGRGDLGLARAYIAGDLEIQGVHPGDPYELLKALADSLVFKLPPPRVMTQIIRSIGVEHLRPIAPPPQEVPPRWRRIAEGLRHSKGRDAEAIHHHYDVSNTFYEWVLGPSMTYTCACYPGLDASLDEAQQNKYRLVFEKLRLKPGDRLLDVGCGWGGMVRYAARHGVQALGVTLSREQTAWAQQAIAVEGLADLAEVRYGDYRDIAEDGFDAVSSIGLLEHIGVRNYASYFGFLQSRLRPGGLLLNHCITRPDNRSEPSARGFIDRYVFPDGELTGSGRIITEAQDVGFEVLHEENLRQHYALTLRDWCANLVAHWEEAVAEVGLPTAKVWGLYMAGSRLAFESGGIQLHQVLAVRPDDRSDAAQLPLRPWWTP

SEQ ID NO: 5 - DNA, 1311 bp, Amycolicicoccus subflavus
atgaaggcagtgttgacggcgtttacggctccccaactcgaaaggatgaacgtcgctgagatactcagcgcggtactcgggcgagatttcccgatccggttcactgcgtacgacggcagcgcgctcggccccgaaaccgcccgctacggcttgcacctcacgacgccgcgcgggctgacctacctcgctaccgcgcccggtgatctcgggctcgcacgcgcgtacgtgtccggcgacctcgaggtcagtggggttcatcagggtgacccgtacgagataatgaagatcctcgcgcatgacgtccgggtgcggcggccctcgccagcaacgatcgcttcgatcatgcggtccctcggctgggaacgcttgcgaccggtcgcgccgcccccgcaagagaacatgccccgttggcgccggatggcccttggcctgctgcactcgaagagccgtgatgctgcggcaatccaccatcattacgacgtgtcgaacgagttttacgagcacatcctcggcccgtcgatgacgtacacatgcgcggcctaccccagcgcagacagttccctggaggaagcacaggacaacaagtaccgactcgtcttcgagaaacttggcctgaaagccggggatcgcctgcttgacgtcgggtgcgggtggggcggcatggtgcggttcgccgctaagcgcggcgttcatgtcatcggtgcgacattgtcccgcaaacaggcggaatgggctcagaagatgattgcccatgaaggattgggcgatctggcggaagtccgtttctgcgactaccgcgatgtcacagaggcgggcttcgacgcagtgtcgtcgatcggcctcactgaacacatcggtttggcgaactacccgtcgtacttcggcttcctgaaggacaagttgcggccaggcggacgactgctgaaccattgcatcactcgcccgaacaaccttcaaagcaaccgcgcaggtgacttcattgaccggtacgttttccctgacggagagctcgccggacctggcttcatcatttcagctgtccacgacgccggtttcgaggtgcggcacgaagagaacctccgcgagcactacgcactgacgctgcgggactggaaccgcaacctcgctcgcgactgggacgcgtgtgtgcacgcctccgacgagggcaccgcccgcgtctggggactgtacatttccggttcacgagtcgcgtttgaaacgaactcgattcagctgcaccaggtcctggcggtcaaaaccgcgcggaatggcgaagcgcaggtcccgttgggtcagtggtggacccgctga

SEQ ID NO: 6 - protein, 436 aa, Amycolicicoccus subflavus
MKAVLTAFTAPQLERMNVAEILSAVLGRDFPIRFTAYDGSALGPETARYGLHLTTPRGLTYLATAPGDLGLARAYVSGDLEVSGVHQGDPYEIMKILAHDVRVRRPSPATIASIMRSLGWERLRPVAPPPQENMPRWRRMALGLLHSKSRDAAAIHHHYDVSNEFYEHILGPSMTYTCAAYPSADSSLEEAQDNKYRLVFEKLGLKAGDRLLDVGCGWGGMVRFAAKRGVHVIGATLSRKQAEWAQKMIAHEGLGDLAEVRFCDYRDVTEAGFDAVSSIGLTEHIGLANYPSYFGFLKDKLRPGGRLLNHCITRPNNLQSNRAGDFIDRYVFPDGELAGPGFIISAVHDAGFEVRHEENLREHYALTLRDWNRNLARDWDACVHASDEGTARVWGLYISGSRVAFETNSIQLHQVLAVKTARNGEAQVPLGQWWTR

SEQ ID NO: 7 - DNA, 1308 bp, Corynebacterium glutamicum
atgagtaacgccgtagcgcaggacctcatgaccatcgccgacatcgtcgaggccacgaccactgcacccatcccattccacatcactgccttcgatggaagcttcactggccctgaagatgctccctaccagctgtttgttgccaacacggatgcagtatcctacatcgcaacagcgccaggagatttgggtttggcacgtgcctacctcatgggagacctcatcgtggaaggtgagcatcccggccatccttatgggatctttgatgcgttgaaggagttctaccgctgcttcaaacgcccagatgcatccaccaccttgcagatcatgtggactctgcggaaaatgaatgccttaaaattccaggaaattccaccaatggaacaagcccctgcatggcgtaaagcactgatcaacgggctagcatccaggcactcgaaatcccgcgacaagaaagccattagctaccactacgacgtgggcaatgagttctactccctgtttttagatgattccatgacctatacctgcgcgtattatccaacgccagaatcaagtttggaagaagcccaagaaaacaaataccgcctcatctttgaaaaactgcgtctgaaagaaggcgatcgcctcctagacgtgggatgcggttggggaggcatggtccgctacgccgccaaacacggtgtgaaagccatcggagttacgctgtctgaacagcaatatgagtggggtcaagcagagatcaaacgccaaggtttggaagacctcgcggaaattcgcttcatggattaccgcgatgttccagaaactggattcgatgcgatctcagcaatcggcatcattgaacacatcggtgtgaacaactatcccgactactttgaattgctcagcagcaaactcaaaacaggcggactgatgctcaaccacagcatcacctacccagacaaccgcccccgccacgcaggtgcatttattgatcgctacattttccccgacggtgaactcactggctctggcaccctgatcaagcacatgcaggacaacggtttcgaagtgctgcacgaagaaaacctccgctttgattaccaacgcaccctgcacgcgtggtgcgaaaacctcaaagaaaattgggaggaagcagttgaactcgccggtgaacccactgcacgactctttggcctgtacatggcaggttcggaatggggatttgcccacaacatcgtccagctgcaccaagtactgggtgtgaaactcgatgagcagggaagtcgcggagaagttcctgaaagaatgtggtggactatctaa

SEQ ID NO: 8 - protein, 435 aa, Corynebacterium glutamicum
MSNAVAQDLMTIADIVEATTTAPIPFHITAFDGSFTGPEDAPYQLFVANTDAVSYIATAPGDLGLARAYLMGDLIVEGEHPGHPYGIFDALKEFYRCFKRPDASTTLQIMWTLRKMNALKFQEIPPMEQAPAWRKALINGLASRHSKSRDKKAISYHYDVGNEFYSLFLDDSMTYTCAYYPTPESSLEEAQENKYRLIFEKLRLKEGDRLLDVGCGWGGMVRYAAKHGVKAIGVTLSEQQYEWGQAEIKRQGLEDLAEIRFMDYRDVPETGFDAISAIGIIEHIGVNNYPDYFELLSSKLKTGGLMLNHSITYPDNRPRHAGAFIDRYIFPDGELTGSGTLIKHMQDNGFEVLHEENLRFDYQRTLHAWCENLKENWEEAVELAGEPTARLFGLYMAGSEWGFAHNIVQLHQVLGVKLDEQGSRGEVPERMWWTI

SEQ ID NO: 9 - DNA, 1368 bp, Corynebacterium glyciniphilium
atgagcaggggattcacgccgctgacggtgggacagatcgtggacaaggtcatcacaccgccggcaccgttccgggtgaccgctttcgacggatccaccgcggggccggcagacgcggaactggcactggagatcacatcgccggacgccctggcctatatcgtgaccgcgccgggcgacctcggactggcacgcgcctacatcaccggaagcctccgcgtcaccggtgacgagcccggccacccgtacctcgtctttgaccacctccagcacctttacgaccagatccgacgcccctcggcgaaggacctgctggatatcgcccgctcgctgaaggccatgggggcgatcaaggtgcagccggcaccggagcaggagaccctcccgggctggaagagggccatactcgaggggctgtcccggcactctccggaacgggacaaggaggtcgtgagccgccactacgacgtgggcaatgacttctacgagctcttcctcggcgattccatggcctacacctgtgcctactatcccgagtttgacggtgagaaccaggtcaccggtcccaccggcgggtggcggtacgacgactgggagaaagggccgaccgccaacgggccgttgacccaggcgcaggacaacaagcatcgcctggtcttcgacaagctgcgactcaacccgggtgaccggttgttggacgtcggctgcgggtggggcggtatggtgcggtacgccgcccgccacggcgtgaaggccatcggtgtcacgctgtcccgagagcagtacgagtggggtaaggcgaagatcgaggaggagggtctgcaggacctcgccgaggtccggtgtatggactaccgtgacgtgccggagtccgacttcgacgcggtcagtgccatcggcatcctcgagcacatcggcgtgcccaactacgaggactacttcacccgcctgttcgccaagctgcgcccgggcggtcggatgctgaaccactgcatcacccgtccgcacaaccggaagacgaagaccggccagttcatcgaccgctacatcttccccgacggtgagctgaccggctcgggccggatcatcacgatcatgcaggacaccggattcgacgtcgtccacgaggagaatctgcgaccgcactaccagcgcacgttgcatgactggtgtgaactgttggccaccaactgggaccaggccgtccatctcgtgggcgaggagacggctcgtctgttcggcctgtacatggcggggtcggaatggggtttcgaacacaacgtgatccagctccaccaggttctcggcgtgaagccggacgcggcaggcagttccggggtgccggtccgccagtggtggaggtcctga

SEQ ID NO: 10 - protein, 455 aa, Corynebacterium glyciniphilium
MSRGFTPLTVGQIVDKVITPPAPFRVTAFDGSTAGPADAELALEITSPDALAYIVTAPGDLGLARAYITGSLRVTGDEPGHPYLVFDHLQHLYDQIRRPSAKDLLDIARSLKAMGAIKVQPAPEQETLPGWKRAILEGLSRHSPERDKEVVSRHYDVGNDFYELFLGDSMAYTCAYYPEFDGENQVTGPTGGWRYDDWEKGPTANGPLTQAQDNKHRLVFDKLRLNPGDRLLDVGCGWGGMVRYAARHGVKAIGVTLSREQYEWGKAKIEEEGLQDLAEVRCMDYRDVPESDFDAVSAIGILEHIGVPNYEDYFTRLFAKLRPGGRMLNHCITRPHNRKTKTGQFIDRYIFPDGELTGSGRIITIMQDTGFDVVHEENLRPHYQRTLHDWCELLATNWDQAVHLVGEETARLFGLYMAGSEWGFEHNVIQLHQVLGVKPDAAGSSGVPVRQWWRS

SEQ ID NO: 11 - DNA, 1284 bp, Knoella aerolata
atgagccacacgaccgatgagatccgcacggtcgccgacctcgtcgacgaggtggtcgtcggcccgctgccggtgcgggtcacggcctacgacgggtcgaagacggggccggacagcgccccgcgaaccatccacatcgccaaccagcgagcggtcgcctacctcgccaccgcgcccggggacctcggcatggcccgcgcctacaccaccggtgacctcgtcgtcgagggcgtgcacccgggcaacccctacgaggccctggtcgacctcgaacgtgtgcacttccgccgcccggacccgcggctgctcctcgacctcgcgcgcatcgtcgggccacgcaacctcgcgcccccgcccccgccgccgcaggaggctgtgccgaggtggcggcgggtggccgagggcctgcgccactcgtacgggcgggacagcgaggcgatccgccaccactacgacgtctccaaccacttctacgagcaggtgctcggcccgagcatgacctacacctgcgcggtcttccccgaccacgacaccgggctcgacgaggcgcaggaggagaagtaccgcctcgtcttcgagaagctcgcgctgcgtcccggtgaccggttgctcgacatcggctgcgggtggggcgggatggtccggtacgccgcacggcggggggtgcgagcgctcggcgtgacactgtccggtgagcaggcggcgtgggcacaggtcgccatcgcccgcgaggggctgggggagctcgccgccgtccggcacgaggactaccgccacgtcgccgagaccgggttcgacgccatctcctcgatcggcatcaccgagcacatcggggtgcgcaactaccccacgtacttcgactggatgctccaccacgtcaagccgggagggctcgtgctcaaccactgcatcaccagacccgagaaccgggccaagagcgtcggccggttcatcgaccgctacatcttccccgacggcgagctcaccgggtccggccggatcatcacgaccatgcaggacaacggtttcgaggtcgtgcactccgagaacctgcgagagcactacgccctcaccctggcggcctggggcgagaacctcgtcgagcactgggcctcctgcgtggccgacgtgggggaggggacggcgaaggtctggggcctctacctcgcgggctcgcgtcgtggcttcgagcgcaacgtcgtccagctgcaccaggtgctggccgcgaggccggtgccgtcccgactcccgcaggtgccgctgcgccagtggtggacctcgtga

SEQ ID NO: 12 - protein, 427 aa, Knoella aerolata
MSHTTDEIRTVADLVDEVVVGPLPVRVTAYDGSKTGPDSAPRTIHIANQRAVAYLATAPGDLGMARAYTTGDLVVEGVHPGNPYEALVDLERVHFRRPDPRLLLDLARIVGPRNLAPPPPPPQEAVPRWRRVAEGLRHSYGRDSEAIRHHYDVSNHFYEQVLGPSMTYTCAVFPDHDTGLDEAQEEKYRLVFEKLALRPGDRLLDIGCGWGGMVRYAARRGVRALGVTLSGEQAAWAQVAIAREGLGELAAVRHEDYRHVAETGFDAISSIGITEHIGVRNYPTYFDWMLHHVKPGGLVLNHCITRPENRAKSVGRFIDRYIFPDGELTGSGRIITTMQDNGFEVVHSENLREHYALTLAAWGENLVEHWASCVADVGEGTAKVWGLYLAGSRRGFERNVVQLHQVLAARPVPSRLPQVPLRQWWTS

SEQ ID NO: 13 - DNA, 1323 bp, Mycobacterium austroafricanum
ttgacgacatttcgggacggcgcggccgacaccggcctgcacggagaccgcaagctcaccctggcggaggtcttggaggtcttcgcctcgggccgactgcctctgaagttcacggcgtacgacggcagcagcgcgggcccggacgacgccacgctcgggctggacctgctgaccccccgcgggaccacgtacctcgcaacggctcccggcgatctcggcctggcccgggcctacgtctccggtgacctgcagttgcagggggtgcaccctggcgacccgtacgacctgctcaacgcactggtgcagaaactggacttcaagcgaccgtccgcccgggtgctggcgcaggtcgtccgatcgatcgggatcgagcacctgaaaccgatcgcgccaccgccgcaggaggcgctgccgcggtggcggcgcatcgcagaaggactgcggcacagcaagacccgtgacgccgacgcgatccaccaccattacgatgtctccaacaccttctacgagtgggtgctcgggccgtcgatgacctacacctgcgcctgctacccgcatcccgacgccaccctcgaggaggcgcaggagaacaaatatcggctggtgttcgagaaactgcgcctcaagccgggcgaccgccttctcgacgtgggttgcgggtggggcggaatggtgcgctacgcggcccgtcacggcgtcaaggcgatcggggtgacgctgtccagggagcaggcgcagtgggcacgcgccgccatcgaacgggacggcctgggtgacctcgccgaggtccgccacagcgactaccgcgatgtgcgcgagtcccagttcgacgccgtgtcttcgctggggctcaccgagcacatcggggtcgccaactatccgtcgtacttccggttcctcaagtcgaagttgcgcccgggcggcctactgctcaaccactgcatcacccggcacaacaatcgcaccggccccgccgccgggggattcatcgaccggtatgtgttcccggacggggagctgaccggatcgggccggatcatcaccgagatccaggacgtcggtttggaggtgatgcacgaagagaacctgcgccggcactatgcgctgacacttcgggactggtgccggaatctggtgcagcactgggacgaagcggtcgcagaggtcggcctgcccaccgccaaggtgtggggtctgtacatggctgcctcgcgggtcggcttcgagcagaacagcattcagctgcatcaggtactggcggtgaagctcgacgaacgtggcggggacggcggtttgccgttgcggccctggtggaccgcgtag

SEQ ID NO: 14 - protein, 440 aa, Mycobacterium austroafricanum
LTTFRDGAADTGLHGDRKLTLAEVLEVFASGRLPLKFTAYDGSSAGPDDATLGLDLLTPRGTTYLATAPGDLGLARAYVSGDLQLQGVHPGDPYDLLNALVQKLDFKRPSARVLAQVVRSIGIEHLKPIAPPPQEALPRWRRIAEGLRHSKTRDADAIHHHYDVSNTFYEWVLGPSMTYTCACYPHPDATLEEAQENKYRLVFEKLRLKPGDRLLDVGCGWGGMVRYAARHGVKAIGVTLSREQAQWARAAIERDGLGDLAEVRHSDYRDVRESQFDAVSSLGLTEHIGVANYPSYFRFLKSKLRPGGLLLNHCITRHNNRTGPAAGGFIDRYVFPDGELTGSGRIITEIQDVGLEVMHEENLRRHYALTLRDWCRNLVQHWDEAVAEVGLPTAKVWGLYMAASRVGFEQNSIQLHQVLAVKLDERGGDGGLPLRPWWTA

SEQ ID NO: 15 - DNA, 1323 bp, Mycobacterium qilvum
atgacgacttttcgggaacataccgacagttcggcgtccgacccggatcggaaactcactttggcagaggtgttggagatcttcgccgcgggtcgccgtccgctgaagttcaccgcctatgacggaagtagttgcgggcctgaggatgcgacactgggcctcgacctgctgaccccgcggggcacgacctacctggccacggcgccgggtgatctcggcctggcgcgggcctacatcgccggcgatctgcgcctcagtggtgtgcatcccggcgatccccatgacctgctcacggcgctgacggaacgcctggagtacaggcgtccgccggtgcgagtgctggccaatgttctgcgctccatcgggatcgagcacctcaagcccgtcgcgccgccaccccaggagcacctgccgcggtggcggcggatcgcagaggggttgcggcacagcaagacccgtgacgctgaggccatccagcaccactacgacgtctcgaacacgttctactcatgggtcctgggtccgtcgatgacctacacctgcgcctgctatccacacccggatgccacgctggaggaggcgcaggagaacaagtaccggctggtgttcgagaagcttcgactcaagcccggtgaccggctgctcgacgtcggttgcggctggggcggaatggtccgctacgccgcccggcacggggtcaaggtcctgggggtgacgctgtcgaaggagcaggcgcagtgggcggccgacgcagtcgagcgggacggcctgggtgagttggccgaggtccgccacggcgactaccgcgacgtgcgcgagtcgcacttcgacgcagtgtcctcgctcgggctcaccgagcacatcggcgtcgcgaactacccgtcgtacttccgcttcctgaagtcgaaactgcggccgggtggcctgctgctcaaccactgcatcacccgaaacaacaaccggagtcacgccaccgcaggcggattcatcgatcgctatgtctttcccgacggggagctgacggggtcggggcgaatcatcaccgaaatgcaggacgtcggactcgaggtcgtgcacgaggagaatctgcgtcaccactacgcgctgacgctgcgcgactggagccgcaacctggtcgcgcactgggacgacgcggtgaccgaggtcggtctgccgactgccaaggtgtggggcctctacatcgccgcgtcgcgagtcggcttcgagcagaacgccattcagctgcaccaggtgctgtcggtcaagctcgacgagcgtggctcggacggcggactgccgttacgaccctggtggaacgcctag

SEQ ID NO: 16 - protein, 436 aa, Mycobacterium qilvum
MTTFREHTDSSASDPDRKLTLAEVLEIFAAGRRPLKFTAYDGSSCGPEDATLGLDLLTPRGTTYLATAPGDLGLARAYIAGDLRLSGVHPGDPHDLLTALTERLEYRRPPVRVLANVLRSIGIEHLKPVAPPPQEHLPRWRRIAEGLRHSKTRDAEAIQHHYDVSNTFYSWVLGPSMTYTCACYPHPDATLEEAQENKYRLVFEKLRLKPGDRLLDVGCGWGGMVRYAARHGVKVLGVTLSKEQAQWAADAVERDGLGELAEVRHGDYRDVRESHFDAVSSLGLTEHIGVANYPSYFRFLKSKLRPGGLLLNHCITRNNNRSHATAGGFIDRYVFPDGELTGSGRIITEMQDVGLEVVHEENLRHHYALTLRDWSRNLVAHWDDAVTEVGLPTAKVWGLYIAASRVGFEQNAIQQVLSVKLDERGSDGPLRPWWNA

SEQ ID NO: 17 - DNA, 1263 bp, Mycobacterium indicus pranii
atggccgagatcctggaggtcttcgccgccaccggccgacatccgctgaagttcaccgcctacgacggcagcatcgccggcaacgaggacgccgaactgggcctggaccttcgcagcccccgcggcgccacctatctggcgaccgcccccggcgaactcggcctcgcccgcgcctacgtgtcgggcgacctgcaggcctacggcgtccatcccggcgacccgtaccaactgctcaagacgctcaccgatcgggtggaattcaagcggcccccggtgcgggtgctggccaacgtcgtgcggtcgctggggttcgagcggttgctgccggtcgcgccgcccccgcaggaggcgctgccccggtggcggcgcatcgccgacgggctgatgcacacgaggacccgcgacgccgaggccatccaccaccactacgacgtgtccaacaccttctacgaattggtgttggggccgtcgatgacctacacctgcgcggtgtatcccgatgccgacgcgacactcgaacaggcgcaggagaacaagtaccggctgatcttcgagaagctgcggctgaaggcgggcgaccggctgctcgacgtcggctgcggctggggcggcatggtgcgctacgcggcccggcgcggcgtccgggccaccggcgccaccctgtcggccgaacaggcgaagtgggcgcagaaggcgatcgccgaggaaggccttgcggacctggccgaggtgcgccacaccgactatcgggacgtgggcgaggcggcgttcgacgccgtgtcctcgatcgggctgaccgagcacatcggcgtcaagaattaccccgcctacttcggcttcttgaagtcgaagctgcgcaccggcggcctgctgctcaatcactgcatcacccgccacgacaacacgtcgacgtcgttcgcgggcggattcaccgatcgctatgtcttcccggacggggagctgaccggctcgggccgcatcacctgcgacgtccaggactgcggcttcgaggtgctgcacgcggagaacttccgccaccactacgcgatgacgctgcgcgactggtgccgcaatctggtcgagaactgggacgccgcggtcagcgaggtcggcctaccgaccgcgaaggtctggggcctgtacatggcggcgtcacgggttgcgttcgagcagaacaaccttcagctgcatcacgtgctggcggccaagaccgacgcgcggggcgacgacgacctgccgctgcggccgtggtggacggcctga

SEQ ID NO: 18 - protein, 420 aa, Mycobacterium indicus pranii
MAEILEVFAATGRHPLKFTAYDGSIAGNEDAELGLDLRSPRGATYLATAPGELGLARAYVSGDLQAYGVHPGDPYQLLKTLTDRVEFKRPPVRVLANVVRSLGFERLLPVAPPPQEALPRWRRIADGLMHTRTRDAEAIHHHYDVSNTFYELVLGPSMTYTCAVYPDADATLEQAQENKYRLIFEKLRLKAGDRLLDVGCGWGGMVRYAARRGVRATGATLSAEQAKWAQKAIAEEGLADLAEVRHTDYRDVGEAAFDAVSSIGLTEHIGVKNYPAYFGFLKSKLRTGGLLLNHCITRHDNTSTSFAGGFTDRYVFPDGELTGSGRITCDVQDCGFEVLHAENFRHHYAMTLRDWCRNLVENWDAAVSEVGLPTAKVWGLYMAASRVAFEQNNLQLHHVLAAKTDARGDDDLPLRPWWTA

SEQ ID NO: 19 - DNA, 1314 bp, Mycobacterium phlei
atgacggcgatcaaagagaacccggtcctgacttcggccaggaagctgtccctggccgagattctggaaatccttgccgggggcgaactcccggtgcgtttcacggcctacgacggcagctcggcgggcccggcggactccccgctcggcctggagctgctgaccccgcgcggcaccacctatctggccaccgccccgggcgatctcgggctggcacgcgcctacatcgccggtgacctgcagccgcacggcgtgcatccgggcgatccgtacgagctgctcaaggccctgtcggagaagatggagttcaagcggccgcccgcgaaggtgctggccaacatcgtgcgctccatcggtatcgagcacctcaagccgatcgcaccgccgccgcaggaggcgcagccgcgctggcgccggatcgcggaagggttgcggcacagcaagactcgcgacgccgaggcgatccaccaccactacgacgtgtccaacacgttctacgagtgggtgctcggcccgtcgatgacctacacctgcgcgtgctacccggacgtcgacgcaaccctggagcaggcgcaggagaacaagtaccgcctggtgttcgagaagctgcgcctgaagccgggcgaccggctgctcgacgtgggctgcggctggggcggcatggtgcgctacgccgcccagcacggggtcaaggccatcggcgtcacgctgtctcgggagcaggcgacgtgggcgcagaaggcgatcgccgagcaggggctcagcgatctggccgaggtccgccacggcgactaccgcgacattcgcgagtccgggttcgacgcggtgtcctcgatcgggctgaccgagcacatcggcgtggccaactacccgtcgtacttccggttcctgcagtccaagctgcgtgtcggcgggctgctgctcaaccactgcatcacccggccggacaacaagtcgcaggccagcgcgggcgggttcatcgaccgctacgtgttccccgacggggagctcaccgggtccggccgcatcatcgccgcggcccaggacgtcggcctcgaggtggtgcacgaggagaacctgcgccagcactacgcgatgacgctgcgcgactggtgccgcaacctcgtcgagcactgggacgaggcggtcgccgaggtcggcctggaacgcgccaagatctggggcctgtacatggccggctcccggctcggcttcgagacgaacatcgtgcagctgcaccaggtgctggcggtcaagctggaccgcaggggcggcgacggcgggctgccgttgcgcccgtggtggacgccctag

SEQ ID NO: 20 - protein, 437 aa, Mycobacterium phlei
MTAIKENPVLTSARKLSLAEILEILAGGELPVRFTAYDGSSAGPADSPLGLELLTPRGTTYLATAPGDLGLARAYIAGDLQPHGVHPGDPYELLKALSEKMEFKRPPAKVLANIVRSIGIEHLKPIAPPPQEAQPRWRRIAEGLRHSKTRDAEAIHHHYDVSNTFYEWVLGPSMTYTCACYPDVDATLEQAQENKYRLVFEKLRLKPGDRLLDVGCGWGGMVRYAAQHGVKAIGVTLSREQATWAQKAIAEQGLSDLAEVRHGDYRDIRESGFDAVSSIGLTEHIGVANYPSYFRFLQSKLRVGGLLLNHCITRPDNKSQASAGGFIDRYVFPDGELTGSGRIIAAAQDVGLEVVHEENLRQHYAMTLRDWCRNLVEHWDEAVAEVGLERAKIWGLYMAGSRLGFETNIVQLHQVLAVKLDRRGGDGGLPLRPWWTP

SEQ ID NO: 21 - DNA, 1263 bp, Mycobacterium tuberculosis
atggccgagatcctggagatcttcaccgcgaccgggcaacacccgctgaagttcaccgcgtatgacggcagcaccgcgggacaagacgacgccacactgggcctggatcttcggacgccccgcggcgccacctacttagctaccgctcccggcgaactcggcctggcccgcgcttatgtgtcgggtgacctacaggcacacggagtacatcccggcgatccgtacgaactgctcaaaacgctgaccgaaagggtcgacttcaaacggccgtcggcgcgggtgctggctaatgtggtgcgctcgatcggcgttgagcacatactgcccatcgcgccgccaccccaggaggcgcgaccccggtggcgtcgaatggctaatggcttgctgcacagcaagacccgtgacgccgaggctatccatcaccactacgacgtctccaacaacttctacgagtgggtgctcgggccatcgatgacctacacgtgcgcggtgtttccgaacgctgaggcttcgctggagcaggcccaagagaacaaataccgactcattttcgaaaagctacggctagagccgggtgaccggctactcgacgtcggctgcggctggggcggcatggtgcgctacgccgcccgacgcggtgtccgggtgatcggcgccacgctctcggccgagcaggccaagtggggccagaaagcagtcgaggacgagggattgagcgacctcgcgcaggtgcggcattccgactaccgcgacgtagccgagaccggtttcgacgccgtttcttcgatcgggctaaccgagcacatcggcgtcaagaattacccgttctacttcgggtttctcaagtcgaagttgcgcaccggcggcttgctgctcaatcactgcatcacccgccacgacaacaggtcgacgtcctttgccggcgggttcaccgaccgttacgttttccccgacggggagctgacgggctcgggacgtattaccaccgagatccagcaggtcggcttggaagtgctgcacgaggagaacttccgccatcactacgcgatgacgctgcgcgactggtgcggcaacctcgtcgaacactgggacgacgcggtcgccgaggtcggtctgccgaccgccaaggtgtggggcctgtacatggcggcttcgcgggtggccttcgaacgaaacaacctgcagctacatcacgtattggcgaccaaggtggacccccggggcgacgacagcttgccactgcggccctggtggcagccctag

SEQ ID NO: 22 - protein, 420 aa, Mycobacterium tuberculosis
MAEILEIFTATGQHPLKFTAYDGSTAGQDDATLGLDLRTPRGATYLATAPGELGLARAYVSGDLQAHGVHPGDPYELLKTLTERVDFKRPSARVLANVVRSIGVEHILPIAPPPQEARPRWRRMANGLLHSKTRDAEAIHHHYDVSNNFYEWVLGPSMTYTCAVFPNAEASLEQAQENKYRLIFEKLRLEPGDRLLDVGCGWGGMVRYAARRGVRVIGATLSAEQAKWGQKAVEDEGLSDLAQVRHSDYRDVAETGFDAVSSIGLTEHIGVKNYPFYFGFLKSKLRTGGLLLNHCITRHDNRSTSFAGGFTDRYVFPDGELTGSGRITTEIQQVGLEVLHEENFRHHYAMTLRDWCGNLVEHWDDAVAEVGLPTAKVWGLYMAASRVAFERNNLQLHHVLATKVDPRGDDSLPLRPWWQP

SEQ ID NO: 23 - DNA, 1323 bp, Mycobacterium vanbaalenii; identical to SEQ ID NO: 13

SEQ ID NO: 24 - protein, 440 aa, Mycobacterium vanbaalenii; identical to SEQ ID NO: 14

SEQ ID NO: 25 - DNA, 1302 bp, Rhodococcus opacus
atgacaactctgaaagcttcacgctcccaggaccacaagctgaccatcgcagagattctcgaaactctgtccgacggcatgctccccctgcggttctccgcctacgacggcagcgccgccggcccggaggacgccccctacggtctccacctcaagacgacccgaggcaccacctacctggcgaccgcccccggcgacctcggcatggcccgggcctacgtgtccggcgacctcgaggcccgcggcgtccaccccggcgacccgtacgagatcctccgcgtgatgggcgacgaactgcacttccgccgtccgtccgcgctcacgctcgccgccatcacgcgctcgctcggctgggatctgctgcgccccatcgcccctcccccgcaggagcatctcccgcggtggcgtcgagtcgcggaagggttgcggcactccaagtcccgcgacgccgaggtcatccaccaccactacgacgtctcgaacaccttctacgagtatgtcctcggcccgtccatgacgtacacgtgcgcctgctacgagaacgccgagcagaccctcgaagaggcacaggacaacaagtaccgcctcgtcttcgagaagctcggcctccagcccggcgaccgactgctcgacatcggttgcggctggggatcgatggtccggtacgccgcccgccgcggcgtcaaggtcatcggcgccaccctgtcccgagagcaggccgaatgggcacagaaggccatcgccgaagaaggactgtccgacctcgccgaggtccggttctccgactaccgtgacgtccccgagaccggattcgacgccatctcctcgatcggcctgaccgagcacatcggcgtcggcaactaccccgcctacttcggactgctgcagagcaagctccgcgagggcggccggctgctgaaccactgcatcacccggcccgacaaccagagtcaggcacgcgcgggcggcttcatcgaccggtacgtcttccccgacggcgaactcaccggctccggacgcatcatcaccgagatccagaacgtcggactcgaggtgcggcacgaggagaatctgcgcgagcactacgcactcaccctcgccggctggtgccagaacctcgtcgacaactgggacgcctgcgtcgccgaggtcggcgaaggcaccgcacgtgtgtggggtctctacatggccgggtcgcgactgggcttcgaacgcaacgtcgttcagctgcaccaggtcctcgccgtcaagctcggacccaagggcgaggcgcatgtgccgctgcgtccgtggtggaagtag

SEQ ID NO: 26 - protein, 433 aa, Rhodococcus opacus
MTTLKASRSQDHKLTIAEILETLSDGMLPLRFSAYDGSAAGPEDAPYGLHLKTTRGTTYLATAPGDLGMARAYVSGDLEARGVHPGDPYEILRVMGDELHFRRPSALTLAAITRSLGWDLLRPIAPPPQEHLPRWRRVAEGLRHSKSRDAEVIHHHYDVSNTFYEYVLGPSMTYTCACYENAEQTLEEAQDNKYRLVFEKLGLQPGDRLLDIGCGWGSMVRYAARRGVKVIGATLSREQAEWAQKAIAEEGLSDLAEVRFSDYRDVPETGFDAISSIGLTEHIGVGNYPAYFGLLQSKLREGGRLLNHCITRPDNQSQARAGGFIDRYVFPDGELTGSGRIITEIQNVGLEVRHEENLREHYALTLAGWCQNLVDNWDACVAEVGEGTARVWGLYMAGSRLGFERNVVQLHQVLAVKLGPKGEAHVPLRPWWK

SEQ ID NO: 27 - DNA, 1317 bp, Streptomyces regensis
ttggcgtcgtcggggccaccgctgcccgccagggcggggtcccgatcggctgactcgacggcgttggacgcgatcctgcgccgcgtgctcggggacgacccgcccgtggccgtgaccgcgttcgacggcacggtggtcggtgacccggactcggcgctgcagctgcacatccgcacgccgacggccctgagctacgtgctcaccgcgcccaacgaactcgggttggcgcgggcctacgtcacgggacatctcgacgtgaccggcgacgtctaccaggtgctgcgcgcactgacgagcgtggccgagaacctcacgacggccgatcggatgtggctggccggccgtctcgcacgggacttcaccgaccggctgcggccggtgccgatccccgtcgaggaggcgccgtcgcggctccgcaggaccgcacgtggcctccggcattccaaggcgcgcgacagcgacgcgatctcccggcactacgacgtctcgaaccgcttctacgagctggtgctcggcccgtcgatggcctacacgtgcgcctgctacccggaggatgcggccacgctggagcaggcacagttccacaagttcgacctcgtgtgccgaaagctcggtctgaagccggggatgcgcctgctcgacgtgggctgcggttggggcggcatggtcgcccacgccgtggagcactacggggtgcgggcgatcggcgtcaccctctcgcgccagcaggcggagtggggacagcgggacctcgaggccaggggcctggccgatcgcggcgagatccgccatctggactaccgcgacgtgcccgagaccgggttcgacgcggtgtcgtccatcgggctcaccgaacacatcggcgcgcggaacctgccgtcgtacttccgcttcctgcactcgaagttgcgtcccggcggacggttgctcaaccactgcatcgtgcgcccgcacacctacgactcccatcggacgggcccgttcatcgaccgctacgtcttcccggacggcgaactcgagggcgtcgggacgatcgtgtcggcgatgcaggaccacgggttcgaggtacggcacgcggagaacctgcgggaacactacgggcgcaccctcgcggcgtggtgcgccaatctcgacgcgcactgggaggcggcggtggccgaggcgggcgtgcagcgggccagggtgtgggcgctgtacatggcggcctcccggctgtcgttcgaacgtcatgagctcgagctgcagcaggtgctcggcgtgaaacccgacgccgcgggcgggtcgtcgatgccgcttcgcccggactggggggtgtga

SEQ ID NO: 28 - protein, 438 aa, Streptomyces regensis
LASSGPPLPARAGSRSADSTALDAILRRVLGDDPPVAVTAFDGTVVGDPDSALQLHIRTPTALSYVLTAPNELGLARAYVTGHLDVTGDVYQVLRALTSVAENLTTADRMWLAGRLARDFTDRLRPVPIPVEEAPSRLRRTARGLRHSKARDSDAISRHYDVSNRFYELVLGPSMAYTCACYPEDAATLEQAQFHKFDLVCRKLGLKPGMRLLDVGCGWGGMVAHAVEHYGVRAIGVTLSRQQAEWGQRDLEARGLADRGEIRHLDYRDVPETGFDAVSSIGLTEHIGARNLPSYFRFLHSKLRPGGRLLNHCIVRPHTYDSHRTGPFIDRYVFPDGELEGVGTIVSAMQDHGFEVRHAENLREHYGRTLAAWCANLDAHWEAAVAEAGVQRARVWALYMAASRLSFERHELELQQVLGVKPDAAGGSSMPLRPDWGV

SEQ ID NO: 29 - DNA, 1272 bp, Thermobifida fusca
atgcgactggcggaggtattcgaacgtgtcgtcggacccgatgcgcccgtccacttccgggcctacgacggcagcactgcgggagatccacgcagtgaagtcgctatcgtggttcgccacccggcagccgtcaactacatcgtccaagcgccgggagcactcggtttgacccgcgcctacgtggcgggatacctcgacgtcgaaggggacatgtacaccgcgctgcgggcaatggccgacgtggtgttccaggaccggccgcggctgtcccccggggaactgctgcggatcatccgcgggatcgggtgggtgaagttcgtcaaccggcttccaccgccgccgcaggaggtgcgccagtcccgcctcgccgccctgggctggcgccactccaagcagcgcgacgccgaagccatccagcaccactacgacgtctccaacgccttctacgccctggtcttgggcgagtcgatgacctacacctgcgcggtctacccgaccgagcaggccacgctggagcaggcacagttcttcaagcacgagctgatcgcccgcaagctcggtcttgcccctgggatacgactgctggatgtggggtgcggctggggcggcatggtcatccacgcggcccgggagcacggggtcaaagccctgggggtgaccctgtccaaagagcaggctgagtgggcgcagaagcggatcgcccacgagggcctgggcgacctggcagaagtccggcacatggactaccgggacctgcccgacggcgagtacgacgcgatcagctcgatcgggttgaccgagcacgtcggcaaaaagaacgtgcccgcctacttcgcgtcgctgtaccgcaagctcgtcccgggaggccgcctgctcaaccactgcatcacccggccccgcaacgacctgccgcccttcaaacgcggcggggtgatcaaccgctacgtcttccccgatggggagctggaagggcccggctggctgcaggcggcgatgaacgacgccgggttcgaaatccgccaccaggagaacctgcgggagcactacgcacggaccctgcgggactggctggccaacctggaccgcaactgggatgccgcggtgcgggaagtgggggagggcacggcccgagtgtggcggctctacatggccgggtgcgtgctcggcttcgaacgcaacgtggtgcaactgcaccagatcctcggggtgaagctcgacgggaccgaggcgcggatgccgctgcgccccgacttcgaaccgccgctgccttaa

SEQ ID NO: 30 - protein, 423 aa, Thermobifida fusca
MRLAEVFERVVGPDAPVHFRAYDGSTAGDPRSEVAIVVRHPAAVNYIVQAPGALGLTRAYVAGYLDVEGDMYTALRAMADVVFQDRPRLSPGELLRIIRGIGWVKFVNRLPPPPQEVRQSRLAALGWRHSKQRDAEAIQHHYDVSNAFYALVLGESMTYTCAVYPTEQATLEQAQFFKHELIARKLGLAPGIRLLDVGCGWGGMVIHAAREHGVKALGVTLSKEQAEWAQKRIAHEGLGDLAEVRHMDYRDLPDGEYDAISSIGLTEHVGKKNVPAYFASLYRKLVPGGRLLNHCITRPRNDLPPFKRGGVINRYVFPDGELEGPGWLQAAMNDAGFEIRHQENLREHYARTLRDWLANLDRNWDAAVREVGEGTARVWRLYMAGCVLGFERNVVQLHQILGVKLDGTEARMPLRPDFEPPLP

SEQ ID NO: 31 - DNA, 1263 bp, Thermomonospora curvata
atgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtgtga

SEQ ID NO: 32 - protein, 420 aa, Thermomonospora curvata
MTLAKVFEELVGADAPVELTAYDGSRAGRLGSDLRVHVKSPYAVSYLVHSPSALGLARAYVAGHLDAYGDMYTLLREMTQLTEALTPKARLRLLAGVLQDPLLRAAASRRLPPPPQEVRTGRTSWFRHTKRRDAKAISHHYDVSNTFYEWVLGPSMTYTCACFPTEDATLEEAQFHKHDLVAKKLGLRPGMRLLDVGCGWGGMVMHAAKHYGVRALGVTLSKQQAEWAQKAIAEAGLSDLAEVRHQDYRDVTEGDFDAISSIGLTEHIGKANLPSYFGFLYGKLKPGGRLLNHCITRPDNTQPAMKKDGFINRYVFPDGELEGPGYLQTQMNDAGFEIRHQENLREHYARTLAGWCRNLDEHWDEAVAEVGEGTARVWRLYMAGSRLGFELNWIQLHQILGVKLGERGESRMPLRPDWGV

SEQ ID NO: 33 - DNA, 1323 bp, Mycobacterium sp. NAZ190054
atgaccacgttccgggaacgtacaagcgagccggccctgtccggcgatcgcaagctcaccctggcagaagtgctggagatcttcgccgcgggccggctaccgctgaagttcaccgcctatgacggcagcagcaccggccctgacgacgccacgctcggcctggatctcctgaccccgcgcggaaccacctacctggcaaccgctcccggcgatctcgggctggcgcgggcctacatcgccggagacctgcggctgaccggtgtacacccgggcgatccctacgagctgctcaccgcgctggcggagaagctcgaatacaagcggccgtccgcgcgggtgctggccaacgtcgtccggtcgatcgggatcgagcacctcaagccgatcgcgccaccaccccaggaggcgatgccgcggtggcgccgcatcgcagaaggactgcgccacagcaagacccgtgatgcggaggccatccaccaccattacgacgtctcgaacacgttctacgagtgggtgctcggcccgtcgatgacgtacacctgcgcgtgctacccgcatcccgacgccaccctcgaggaggcgcaggagaacaagtaccgtctggtcttcgagaagctgcgcctcaagccgggtgaccgtctgctggacgtgggctgcggctggggcggcatggtgcgctacgcggcccgtcacggcgtcaaagcggtcggggtgacgctgtcgacggagcaggcacggtgggcccgtgccgcgatcgaccgggacgggctcggtgacctcgccgaggtccgccacggtgactaccgcgacgtgcgtgaatcgcacttcgacgcggtgtcctcgatcggtctcaccgagcacatcggcgtggccaactacccgtcgtacttccggttcctgaagtccaagcttcgccccggtggcctgctgctcaaccactgcatcacccgacatcacaaccggagtcacgccgccgcaggcggattcatcgaccgttacgtgttccccgacggagagctgaccgggtcgggccggatcatcaccgagatccaggacgtcggccttgaggtgctgcacgaggagaatctgcgccaccactatgcgatgacgctgcgcgactggtgccggaatctggtggagtactgggacgacgcggtcgccgaggtcggcctgcccaccgcgaaggtgtggggtctgtacatggccgcgtcgcgggtcggcttcgagcagaacggcattcagctgcatcaggtgctggcggtcaagctggacgaacgcggcggcgacggcggattgccgttgcgtcagtggtggagcgcctga

SEQ ID NO: 34 - protein, 440 aa, Mycobacterium sp. NAZ190054
MTTFRERTSEPALSGDRKLTLAEVLEIFAAGRLPLKFTAYDGSSTGPDDATLGLDLLTPRGTTYLATAPGDLGLARAYIAGDLRLTGVHPGDPYELLTALAEKLEYKRPSARVLANVVRSIGIEHLKPIAPPPQEAMPRWRRIAEGLRHSKTRDAEAIHHHYDVSNTFYEWVLGPSMTYTCACYPHPDATLEEAQENKYRLVFEKLRLKPGDRLLDVGCGWGGMVRYAARHGVKAVGVTLSTEQARWARAAIDRDGLGDLAEVRHGDYRDVRESHFDAVSSIGLTEHIGVANYPSYFRFLKSKLRPGGLLLNHCITRHHNRSHAAAGGFIDRYVFPDGELTGSGRIITEIQDVGLEVLHEENLRHHYAMTLRDWCRNLVEYWDDAVAEVGLPTAKVWGLYMAASRVGFEQNGIQLHQVLAVKLDERGGDGGLPLRQWWSA

SEQ ID NO: 35 - DNA, 1266 bp, Marinactinospora thermotolerans DSM 45154
atgcggctggcggagatcttcgagcgggtcgtcggggctgacgcccctgttcggttccgcgcctacgacggcagcacctcgggagatcccgaaagcgagatcaccctcgtcgtgcgtaccccggtggcgctcaactacctcgcccagtccccgggggcgctcggcctgacccgggcctacgtcgcggggcacatcgatctcgaaggggacatgtacaccgcgctcaagcggatgtcggagatcgccttcggtgacggggtcaggcttcgcgacttcctcgccatcgccaaggacatcggctgggtcaagttcgtcaaccgcgtaccgccgcctccacaggagatgcgccgctcccggctgctggagcgtgggcggcggcattcgaaggagcgcgacgccgaggtcatccaccaccactacgacgtctccaacgccttctacgagatggtgctggggccgtccatgacctatacctgcgccgtctaccccacaccggtcgcctcccttgaggaggcccagttccacaagtacgagctggtctcgcgcaaactcgggctggccaaggggatgcggctgctggacgtgggctgtggctggggcggcatggtcatccacgccgcccgtgagcacggcgtcaaggcgctgggggtgacgctgtccaaggagcaggccgagtgggcgcagaagaagatcgcccaggaggggctgggcgacctcgccgaggtgcgccacatggactaccgcaacgtccccgacggcgtcttcgaccggatcagctccatcggcctgaccgagcacatcggcaagaagaacgtgccggactacttcgcctcgctgtacgcgaagctgaagcccggcggccggctgctcaaccactgcatcacgcgcccccgcaacgaccttcccgcgatggtgccgggaggggtgatcaaccgctatgtcttccccgacggggagctggaggggcccgggtggctccaggcgcagatgaacgacgccgggttcgagatccggcatcaggagaacctgcgtgagcactacgcgctgacgttgcgtgactggtcggccaacctggaccgcaactgggacgcggcggtcgccgaggtcggcgaggggaccgcgcgggtgtggcggctgtacatggcgggctgtgtgctggggttcgagaagaacgtggtccagctccaccagatcctcggcgtcaagctggacggtgacgactcgggcatgccgctgcgcccggacttcgagccgcggcgcggctga

SEQ ID NO: 36 - protein, 421 aa, Marinactinospora thermotolerans DSM 45154
MRLAEIFERVVGADAPVRFRAYDGSTSGDPESEITLVVRTPVALNYLAQSPGALGLTRAYVAGHIDLEGDMYTALKRMSEIAFGDGVRLRDFLAIAKDIGWVKFVNRVPPPPQEMRRSRLLERGRRHSKERDAEVIHHHYDVSNAFYEMVLGPSMTYTCAVYPTPVASLEEAQFHKYELVSRKLGLAKGMRLLDVGCGWGGMVIHAAREHGVKALGVTLSKEQAEWAQKKIAQEGLGDLAEVRHMDYRNVPDGVFDRISSIGLTEHIGKKNVPDYFASLYAKLKPGGRLLNHCITRPRNDLPAMVPGGVINRYVFPDGELEGPGWLQAQMNDAGFEIRHQENLREHYALTLRDWSANLDRNWDAAVAEVGEGTARVWRLYMAGCVLGFEKNVVQLHQILGVKLDGDDSGMPLRPDFEPRRG

SEQ ID NO: 37 - DNA, 1401 bp, Mycobacterium smegmatis
gtgtctgtggttactactgacgcacaggctgcccatgccgccggcgtctcgcgtcttctggccagctaccgggcgatcccgcccagcgcgacagtgcgccttgcgaaaccgacgtccaacctgttccgcgcccgcgcccgcaccaatgtgaagggtctcgacgtctcgggcctgaccggtgtgatcggtgtcgacccggacgcgcgcaccgccgatgtggcgggcatgtgcacctacgaggacctggtggcggccacgcttccgtacggccttgccccactggtggtgccgcagctcaagaccatcacgctcggtggcgcggtcaccggtctgggcatcgagtccacgtcgttccgcaacggtctgccgcacgaaagtgtcctggagatggacatcttgaccggttcgggcgagatcgtcacggcctcaccggatcagcactcggatctgttccatgcgttccccaattcatatggaacccttggttattccacccggctgcgcatcgaactggagcccgtgcacccgtttgtggcgttgcgccacctgcgctttcactcgatcaccgatctggtcgcggcgatggaccggatcatcgagaccggcgggctggacggtgaacccgtcgactacctcgacggcgtggtgttcagcgcgactgagagttacctgtgtgttggcttcaagacgaaaacgccggggccggtcagcgattacacaggtcagcagatcttctaccggtcgatccagcatgacggcgacaccggcgccgagaaacacgaccggctgaccatccacgactacctgtggcgctgggacaccgactggttctggtgctcacgggcattcggcgctcagcatccggtgatccgcaggttctggccgcggcggctgcgccgcagcagcttctactggaagctggtggcctacgaccagcggtacgacatcgccgaccgtatcgagaagcgcaacgggcgcccgccgcgcgagcgggtggtccaggacgtcgaggtgcccatcgagcggtgcgcggacttcgtcgagtggttcctgcagaatgtgccgatcgagccgatctggctgtgccccctacggttgcgtgacagcgccgacggcggtgcctcgtggcccctgtatccgctgaaggcgcaccacacctacgtcaacatcggtttctggtcatcagtgccggtgggccccgaggagggccacaccaaccgcctcatcgagaaaaaagtcgcggagctggacgggcacaaatctttgtactcggacgcttattacacacgtgacgaattcgacgagctgtacggcggtgaggtctacaacaccgtcaagaagacgtacgacccggattcacgtctgctagacctgtattcgaaggcggtgcaaagacaatga

SEQ ID NO: 38 - protein, 466 aa, Mycobacterium smegmatis
VSVVTTDAQAAHAAGVSRLLASYRAIPPSATVRLAKPTSNLFRARARTNVKGLDVSGLTGVIGVDPDARTADVAGMCTYEDLVAATLPYGLAPLVVPQLKTITLGGAVTGLGIESTSFRNGLPHESVLEMDILTGSGEIVTASPDQHSDLFHAFPNSYGTLGYSTRLRIELEPVHPFVALRHLRFHSITDLVAAMDRIIETGGLDGEPVDYLDGVVFSATESYLCVGFKTKTPGPVSDYTGQQIFYRSIQHDGDTGAEKHDRLTIHDYLWRWDTDWFWCSRAFGAQHPVIRRFWPRRLRRSSFYWKLVAYDQRYDIADRIEKRNGRPPRERVVQDVEVPIERCADFVEWFLQNVPIEPIWLCPLRLRDSADGGASWPLYPLKAHHTYVNIGFWSSVPVGPEEGHTNRLIEKKVAELDGHKSLYSDAYYTRDEFDELYGGEVYNTVKKTYDPDSRLLDLYSKAVQRQ

SEQ ID NO: 39 - DNA, 1380 bp, Agromyces subbeticus
gtgtccgctcctgcgaccgatgcacgaaccgcccacgccgacggcgtggagcgattgctcgagagttatcgggcggtgccggcggccgcatcggtgcggctcgccaagcgcacctcgaacctcttccggtcccgagcggcgacggatgcccctggcctcgacacctccggcctgacccacgtcatcgcggtcgaccccggggcgcgcacggccgacgtcgccggcatgtgcacctacgacgacctcgtcgccgcgacactgccgcatgggctcgcgccactcgtggtgccgcaactgaagaccatcaccctcgggggcgccgtaacgggactcggcatcgagtcgacgtcgttccgcaacggtctgccgcacgagtcggtgctcgagatcgacgtgctcaccggcgcaggcgagatcatcacggcgtcgccgatcgagcacgcagagctgttccgcgccttccccaactcgtacggcaccctcggctacgccgtgcgcctgcgcatcgagctcgagccggtcgagccgttcgtcgcactcacgcaccttcggttccatgcgctcaccgacctcatcgaggcaatggagcgcatcatcgagaccggtcgactcgacggggttgccgtcgattccctcgacggcgtggtgttcagcgctgaagagagctacctgtgcgtcggcacgcagaccgcggcatccggcccggtcagcgactacacccgccagcagatcttctatcgctccatccagcatgacgacggtgcgaagcacgaccggctcaccatgcacgactacctgtggcgctgggacgccgactggttctggtgctcgcaggcgttcggcgcgcagcatccgctgattcgccggttctggccgcggcgataccggcgcagccgctcgtactcgacgctcatgcgcctcgaacggcgattcgacctcggcgatcgcctcgagaagctcaagggccggccggcgcgcgaacgcgtgatccaagacgtcgaggtgccgatcgggcgcaccgtcggcttcctcgaatggttcctcgcgaacgtgccgatcgagccgatctggttgtgcccgctgcgcctgcggggcgaccgcggctggcctctctacccgatccggccgcagcagacctacgtcaacatcggcttctggtcgacggttccggtgggcggctccgagggcgagacgaaccgctcgatcgagcgcgccgtgagcgagttcgacggacacaagtcgctgtactccgactcgtactactcgcgcgaggagttcgaggagctctacggcggcgaggcgtaccgggccgtgaagcggcgatacgaccccgactctcgactgctcgacctctatgcgaaggcggtgcaacggcgatga

SEQ ID NO: 40 - protein, 459 aa, Agromyces subbeticus
VSAPATDARTAHADGVERLLESYRAVPAAASVRLAKRTSNLFRSRAATDAPGLDTSGLTHVIAVDPGARTADVAGMCTYDDLVAATLPHGLAPLVVPQLKTITLGGAVTGLGIESTSFRNGLPHESVLEIDVLTGAGEIITASPIEHAELFRAFPNSYGTLGYAVRLRIELEPVEPFVALTHLRFHALTDLIEAMERIIETGRLDGVAVDSLDGVVFSAEESYLCVGTQTAASGPVSDYTRQQIFYRSIQHDDGAKHDRLTMHDYLWRWDADWFWCSQAFGAQHPLIRRFWPRRYRRSRSYSTLMRLERRFDLGDRLEKLKGRPARERVIQDVEVPIGRTVGFLEWFLANVPIEPIWLCPLRLRGDRGWPLYPIRPQQTYVNIGFWSTVPVGGSEGETNRSIERAVSEFDGHKSLYSDSYYSREEFEELYGGEAYRAVKRRYDPDSRLLDLYAKAVQRR

SEQ ID NO: 41 - DNA, 1428 bp, Amycolicicoccus subflavus
atgacgcctgaagctagtgcggcggcgcacgccgctgcggtggatcgcctcatccatagctatcgggcgattcctgatgacgcgccggtgcggctggcgaagaagacgtcaaacctattccgccacagggaaaagacttctgctcctgggcttgacgtatccggcctggctcgcgtgattgggatcgactcagacactcgcactgccgacgttggcggcatgtgcacatacgaggaccttgtcgcggcgacgctcgaatacgatctggtccccctggtcgtcccgcaactcaaaacgatcactctcggcggcgcggtgacgggcctgggaattgagtccacctcgttccgcaatgggcttccccatgaatctgttctcgaaatggatatcctgacgggcgccggggaggtcgtcacggccggcccggaaggcccccatagcgatttgtactgggggtttccgaattcgtacggcacgctcggctatgcgacgcgcctgcgcatcgaactagaaccggtcgagccgtacgtcgaactcaggcacctgcggttcactagcctcgatgagcttcaggagacacttgacaccgtttcgtacgaacacacgtatgacggggaacccgttcattacgtcgatggagtcatgttctcagccacggaaagctacctcacgcttggccgtcagacgagcgaacccggcccggtcagcgactacaccggaaaccagatctactaccgttcaatacagcacggtggcgctgaaactcccgtcgtcgaccggatgaccattcatgactatctatggcgctgggatactgactggttctggtgctcgcgtgccttcggaacgcaacacccagtggtccggagattctggccacgccgctatcgccgcagcagcttctactggaagctgatcgcgcttgaccgccaggttgggctcgcggacttcatcgaacaacggaagggcaacctcccccgggaacgcgtagtccaggacatcgaggtcccgatcgagaacactgcgagcttcttgcggtggttcttggcgaacgtgccgatcgagccggtatggctatgcccgctgcgcctgcgaaaaacacgcagccccggcctgccttcgccgacgtccccggcttcacgcccatggcccctctatccgctcgagcctcagcgcacatacgtcaatgttggcttctggtcagcggtgccggtcgtggccggccagcccgaggggcacaccaaccggatgatcgagaacgaagtcgatcgccttgacggtcacaaatcgctgtactcagatgcgttttacgagcgaaaagagtttgacgcgctgtacggcggcgatacctatagagaactcaaagagacctacgacccaaacagccggttacttgatctctatgcaaaggcggtgcaaggacgatga

SEQ ID NO: 42 - protein, 475 aa, Amycolicicoccus subflavus
MTPEASAAAHAAAVDRLIHSYRAIPDDAPVRLAKKTSNLFRHREKTSAPGLDVSGLARVIGIDSDTRTADVGGMCTYEDLVAATLEYDLVPLVVPQLKTITLGGAVTGLGIESTSFRNGLPHESVLEMDILTGAGEVVTAGPEGPHSDLYWGFPNSYGTLGYATRLRIELEPVEPYVELRHLRFTSLDELQETLDTVSYEHTYDGEPVHYVDGVMFSATESYLTLGRQTSEPGPVSDYTGNQIYYRSIQHGGAETPVVDRMTIHDYLWRWDTDWFWCSRAFGTQHPVVRRFWPRRYRRSSFYWKLIALDRQVGLADFIEQRKGNLPRERVVQDIEVPIENTASFLRWFLANVPIEPVWLCPLRLRKTRSPGLPSPTSPASRPWPLYPLEPQRTYVNVGFWSAVPVVAGQPEGHTNRMIENEVDRLDGHKSLYSDAFYERKEFDALYGGDTYRELKETYDPNSRLLDLYAKAVQGR

SEQ ID NO: 43 - DNA, 1548 bp, Corynebacterium glutamicum
atgagcggattagttgacccggatagtacttttttaaagaccatcggaaaactgagcaacagcttgtccattggtcgtggagtagatcaaaaagaggtaatccccaaaggctggaacgcccattgggaggcaattacaaagcttaagagaagctttgacgcgattcctgctggggagcgggtgcgtttagctaagaaaacctccaacctgttccgtggacgctccgatgcaggtcacggcctagatgtggcagcgcttgggggagtgattgccattgatccggtcaatgccaccgccgatgtacagggcatgtgcacgtatgaagacctggtagatgccactttaagttatggtctgatgccgttggttgtgcctcaactgaaaaccatcacgcttggtggcgcagtgaccggaatgggcgtggaatccacatccttccgcaacggtttgccacacgaatcagtgctggagatggatatttttaccggcactggtgagatcgtgacttgctcgcccacagaaaatgtcgacctttacagaggttttcccaactcttatggttcgctgggatacgcggtgcggctaaaaattgagctggaaccagtgcaagattacgtccagctgcgccacgtgcgcttcaacgatttagagtctttgaccaaagcgattgaggaagtcgcgtcttctctggagtttgataaccaacccgtcgattaccttgacggcgtggtgttttcacccacggaagcctacttagttcttggcacgcaaacctcacaacctggccccaccagcgattacaccagggatttaagctactaccgctccctgcaacacccagagggcatcacctatgaccgcctgacaatccgcgattacatctggcgctgggacaccgactggttctggtgttcacgcgcattcggcacccaaaaccccgtggtgcgcaaactctggcccagggatctgctgcgctcgagtttctattggaagatcatcggctgggatcgaaaatactccatcgctgatcgcctggaagagcgcaaaggccgcccggctagggaacgggtggtccaagacgtggaagttacgattgataaactgccagaatttttgaaatggttctttgaaagcagcgacatcgagccgctgtggctgtgcccgatcaagcttcgggaggtaccaggtagttcggttggtgctggagaaattttgagctccgctgaagcaatcgactccggtgctgctgaacacccttggccgctgtatcccttgaagaaggacgtgctgtgggtcaacatcggattctggtcctcagtgccggttgatctgatgggctccgatgcaccagagggagcatttaacagagaaatcgaacgcgtcatggcagagctaggcggacataaatcgctgtactccgaagcgttctacaccagggaagactttgaaaaactttatggcggaaccatcccggcgctgctaaaaaagcagtgggatccccacagccgattccccggtttgtatgaaaagacagtaaaaggcgcctag

SEQ ID NO: 44 - protein, 515 aa, Corynebacterium glutamicum
MSGLVDPDSTFLKTIGKLSNSLSIGRGVDQKEVIPKGWNAHWEAITKLKRSFDAIPAGERVRLAKKTSNLFRGRSDAGHGLDVAALGGVIAIDPVNATADVQGMCTYEDLVDATLSYGLMPLVVPQLKTITLGGAVTGMGVESTSFRNGLPHESVLEMDIFTGTGEIVTCSPTENVDLYRGFPNSYGSLGYAVRLKIELEPVQDYVQLRHVRFNDLESLTKAIEEVASSLEFDNQPVDYLDGVVFSPTEAYLVLGTQTSQPGPTSDYTRDLSYYRSLQHPEGITYDRLTIRDYIWRWDTDWFWCSRAFGTQNPVVRKLWPRDLLRSSFYWKIIGWDRKYSIADRLEERKGRPARERVVQDVEVTIDKLPEFLKWFFESSDIEPLWLCPIKLREVPGSSVGAGEILSSAEAIDSGAAEHPWPLYPLKKDVLWVNIGFWSSVPVDLMGSDAPEGAFNREIERVMAELGGHKSLYSEAFYTREDFEKLYGGTIPALLKKQWDPHSRFPGLYEKTVKGA

SEQ ID NO: 45 - DNA, 1458 bp, Corynebacterium glyciniphilium
gtgaccgtcgccggcaggatcactgacgcggtacgcataggaaatggacttgaccagcgagatctagcccccgtcgggtggtacgcacacgaacaggccgtggcgcgactgaaggccagtttcgacgcggtccccgccgggcgtcgcgtgcggctggcgaagaagacgtccaaccttttccgcgggcgttccggcgaggcagtcgggctcgacgtgtcggggctgcacggcgtcatcgccgtcgaccccgttgagggcaccgctgacgtccagggcatgtgcacgtacgaggacctggtggacgtcctgctgccctacggtctggcgcccaccgtcgttccgcagctgaagaccatcactctcggcggtgcggtgaccggcatgggggtggaatccacctccttccgcaacggcctgccgcacgaagccgtcctggaaatggatgtgctcaccggtaccggagacatcctcacctgttcgccgacccagaacaccgacctctaccgcggcttccccaactcctacggttccctgggatacagcgtgcggctgaaggtgcggtgcgaacgggtggaaccctacgtcgacctgcggcatgtacgcttcgatgacgttcagtcgctcaccgacgccctcgacaacatcgtcgtggacaaggagtacgagggtgaacgggtcgactatctcgacggtgtggtcttcagcctggaggagagctacctcgtcctgggacgggcgaccagcgaggccggccccgttagcgactacacccgcgagcgcagttactaccgttctctgcagcatccgtcgggggtcctgcgcgacaagttgaccatccgcgactacctctggcggtgggacgtcgactggttctggtgcaaccgggccttcggtacccagaaccccaccatccgtactctgtggccgcgggatctcctgcggtcgagcttctactggaagatcatcggctgggaccgacgcttcgacatcgcggaccggatcgaggcacacaacgggcgccccgcacgcgagcgcgtcgtccaggacatcgaggtcacccccgacaacctgccggagttcctcacgtggttcttcacccactgcgagatcgagccggtgtggctgtgccccattcgactggccgacgactcgggcgagcggacaccgtggcccctgtacccgctgtcacccggcgacacctgggtcaacgtgggattctggagctcggtgcccgccgacctgatggggaaggacgccccgaccggagccttcaaccgggaggtggagagagtcgtctcggacctcggcggacacaagtcgttgtactccgaggcattctattctgaggaacagttcgccgccctctacggcggtgaacgtcccgcacaactcaaggcggtcttcgacccggatgaccggttccccgggttgtacgagaagaccgtgggcggcgtctga

SEQ ID NO: 46 - protein, 485 aa, Corynebacterium glyciniphilium
VTVAGRITDAVRIGNGLDQRDLAPVGWYAHEQAVARLKASFDAVPAGRRVRLAKKTSNLFRGRSGEAVGLDVSGLHGVIAVDPVEGTADVQGMCTYEDLVDVLLPYGLAPTVVPQLKTITLGGAVTGMGVESTSFRNGLPHEAVLEMDVLTGTGDILTCSPTQNTDLYRGFPNSYGSLGYSVRLKVRCERVEPYVDLRHVRFDDVQSLTDALDNIVVDKEYEGERVDYLDGVVFSLEESYLVLGRATSEAGPVSDYTRERSYYRSLQHPSGVLRDKLTIRDYLWRWDVDWFWCNRAFGTQNPTIRTLWPRDLLRSSFYWKIIGWDRRFDIADRIEAHNGRPARERVVQDIEVTPDNLPEFLTWFFTHCEIEPVWLCPIRLADDSGERTPWPLYPLSPGDTWVNVGFWSSVPADLMGKDAPTGAFNREVERVVSDLGGHKSLYSEAFYSEEQFAALYGGERPAQLKAVFDPDDRFPGLYEKTVGGV

SEQ ID NO: 47 - DNA, 1395 bp, Knoella aerolata
atgagcatggaccggaccggaccggccagggtgcggaccgtgggggagcggcggctgctcgagagcttcgccgccgtccccccgggcgaacgcgtgcggctggccaagcgcacgtccaacctcttccgcgcccgggagggcacctcgacacgcgggctcgacacgagcggactgaccggcgtgcgcgtggtcgacgcaggcaccctcacggccgacgtcgacggaatgtgcacgtacgaggacctcgtcgccgcaacgctgccgctcgggctcgcgccgctcgtcgtgccccagctgcggaccatcaccgtcggcggggcggtcaccggtctcgggatcgagtcgacgtcgttccgcaacgggttgccgcacgagtccgtcctcgagatggacgtcctcacgggtgccggcgagatcgtcactgccacagcggacaacgagcacgccgacctcttccgcggcttccccaactcctacgggtcgctgggctacgcgacgtgcctgcgcatcgagctcgagcgtgtgggtacctgtgtggaggtgaggcacgtccgcttccacgacctcgacgccctgtgcgccgccatcgccgaggtcgtggcgacgagatcgcacgagggcgaggaggtcgaccacgtggacggggtggtcttctcccgcgacgaggcgtacctcacgctgggtcgtcactccgaccggaccggaccgaccagcgactacaccgggcagcaggtctactaccggtcgatccagcacgacggcccctctccacggcgcgacctgctcaccactcacgactacctctggcgctgggacaccgactggttctggtgctcgcgcgccttcggggcccaggacccgcgcgtccggcggtggtggccgcgccggtggcgccggtcgagcgtgtactggaggctcgtggcggcggaccggcgcgtcgggttctcggaccgcctcgaggcacgtcggggcaacccgccgcgggagcgggtggtccaggacgtcgagatcccgctcgggcagaccgcggccttcctccactggttcctcgacgaggtgccgatcgaaccgatctggctgtgcccgttgcgtcttcgcgaccatcagaggtggccgctctatccgctcgagcccggacgcacctacgtcaacgtggggttctggtcgaccgtgccggggcccggaccgggcgaggagctgggcgccaccaaccgcgccatcgagcgccgtgtcgacgaggtcggcggccacaagtccctgtactccgactcctactactcccggtccgacttcgacgccctctacggcggggacgcgtatgccgtgctgaaggccacctacgacccggacgggcggttccctcacctctacgacaaggcggtgcgacacgcatga

SEQ ID NO: 48 - protein, 464 aa, Knoella aerolata
MSMDRTGPARVRTVGERRLLESFAAVPPGERVRLAKRTSNLFRAREGTSTRGLDTSGLTGVRVVDAGTLTADVDGMCTYEDLVAATLPLGLAPLVVPQLRTITVGGAVTGLGIESTSFRNGLPHESVLEMDVLTGAGEIVTATADNEHADLFRGFPNSYGSLGYATCLRIELERVGTCVEVRHVRFHDLDALCAAIAEVVATRSHEGEEVDHVDGVVFSRDEAYLTLGRHSDRTGPTSDYTGQQVYYRSIQHDGPSPRRDLLTTHDYLWRWDTDWFWCSRAFGAQDPRVRRWWPRRWRRSSVYWRLVAADRRVGFSDRLEARRGNPPRERVVQDVEIPLGQTAAFLHWFLDEVPIEPIWLCPLRLRDHQRWPLYPLEPGRTYVNVGFWSTVPGPGPGEELGATNRAIERRVDEVGGHKSLYSDSYYSRSDFDALYGGDAYAVLKATYDPDGRFPHLYDKAVRHA

SEQ ID NO: 49 - DNA, 1392 bp, Mycobacterium austroafricanum
gtgtctgttccttcgaccgacgcacgttctgctcacgccgacggcgtgcagcggcttctcgccagctatcgggcgattccccaagacgccacggtccggctggccaaacccacgtcgaacctcttccgtgcccgcgcgaaaaccaggaccaagggtctggacacgtctgggttgacgaacgtgatcgcggtcgacgcggaggcacgcaccgccgatgtggcagggatgtgcacctacgaagacctggtcgcggccacgctgccgcatggactttcgccgctggtggtgccgcagttgaagacgatcaccctcggcggggcggtcaccggactcgggatcgagtccgcctcgttccgcaacggcctgccacacgaatcggttctcgagatggacgtcctcaccggcaccggtgatgtcgtgcgcgcctcccccgacgagaaccctgacctgtttcgggcgtttccgaattcctatggcacgttgggctattcggttcggctcaagatcgagctggaaccggtgaagccgttcgtcgcgctgcgccacctccgtttccattcgctgtcggctctcatcgaggcgatggaccgcatcgtcgaaaccggcggcctcaacggcgaaccggtggactacctcgacggcgtcgtgttcagtgccgaggagagttacctgtgcgtggggcagcgctccgcgacaccgggcccggtcagcgactacacgggcaagcagatctactaccgctcgattcagcacgacggcccgaccgatggcgccgagaagcacgaccggctgaccatccacgactacctgtggcgctgggacaccgactggttctggtgctcaagggcattcggcgcgcagaacccgcggatccggcgctggtggccgcgccggtaccggcgcagcagtgtgtactggaagctgatcggctacgaccggcgtttcggtatcgccgatcgcatcgagaagcgcaacggccgacccccgcgcgagcgggtggtccaggacatcgaggtgcccatcgagcggaccgtcgagtttctgcagtggtttctcgacaccgtgcccatcgaaccgatctggttgtgcccgttgcggctccgcgacgaccgcgattggcccctgtatccgatccgaccccaccacacctacgtcaacgtgggtttctggtcgtcggtgccggtgggcccggaggagggctacaccaacaggatgatcgaacggaaagtcagcgacctcgacggtcacaaatcgctgtattccgatgcgtactactcgccggaagagtttgattcgctctatggcggggagacgtacaagacggtgaagaagacatacgacccagactctcgtttcctggacctgtacggcaaagcagtggggcggcaatga

SEQ ID NO: 50 - protein, 463 aa, Mycobacterium austroafricanum
VSVPSTDARSAHADGVQRLLASYRAIPQDATVRLAKPTSNLFRARAKTRTKGLDTSGLTNVIAVDAEARTADVAGMCTYEDLVAATLPHGLSPLVVPQLKTITLGGAVTGLGIESASFRNGLPHESVLEMDVLTGTGDVVRASPDENPDLFRAFPNSYGTLGYSVRLKIELEPVKPFVALRHLRFHSLSALIEAMDRIVETGGLNGEPVDYLDGVVFSAEESYLCVGQRSATPGPVSDYTGKQIYYRSIQHDGPTDGAEKHDRLTIHDYLWRWDTDWFWCSRAFGAQNPRIRRWWPRRYRRSSVYWKLIGYDRRFGIADRIEKRNGRPPRERVVQDIEVPIERTVEFLQWFLDTVPIEPIWLCPLRLRDDRDWPLYPIRPHHTYVNVGFWSSVPVGPEEGYTNRMIERKVSDLDGHKSLYSDAYYSPEEFDSLYGGETYKTVKKTYDPDSRFLDLYGKAVGRQ

SEQ ID NO: 51 - DNA, 1392 bp, Mycobacterium gilvum
gtgtctgttgccgtaaccgacgcacgatccgcctacgcccacggcgtgcagcggctggtcgcgagttaccgcgccatccccgccggcgccaccgtccgcctggccaaacccacgtccaacctgttccgcgccagggcgaagagcaccgcggcgggcctcgacacctccggcctgacacatgtgatcgccgtggaccccgagacgcgcaccgccgaggtcgcggggatgtgcacctacgaggacctggtggcggcgacgctgccccacgggctttcaccgctggtggtcccgcaactcaagacgatcaccctcggcggcgccgtcaccgggctcggcatcgagtcggcgtcgttccgcaacggccttccgcacgaatcggtcctggagatggacatcctcaccgggaccggcgacatcgtgcgcgccgcgcccgacgagaatcccgaccttttccgcaccttcccgaattcttatggaacgctgggttactcggttcggctgaagatcgagctggagccggtgaagccgttcgtggcgttacgccatctccgcttccactcactgtcgacactcatcgcgacgatggaccgcatcgtcgacaccgggagtctcgacggtgagcaggtcgactatctcgacggagtggtgttcagcgccgaggagagctacctgtgcgtcggaacacgttccgcgacaccgggtcctgtcagcgactacaccggcgagcacatcttctaccggtcgatccagcacgattgcccgaccgaaggcggacagaagcacgaccggctgacggcgcacgactacttctggcgctgggacaccgactggttctggtgctcaagggcattcggcgcgcagaacccgaaggtccgtcggtggtggccccgacggctccggcgcagcagcttctactggaagctcgtcggctacgaccagcgtttcggcatcgccgaccggatcgagaaacaccacggccggccaccgcgcgaacgcgtcgtccaggacgtcgaggtccccatcgagcgcaccgtcgaattcctgcagtggttcctcgacacgatcccgatagagccgctctggttgtgcccgttgcgacttcgcgatgacaacagctggtcgctgtacccgctccggccccatcgcacgtatgtcaacgtgggattctggtcgtcggtgcccgtcgggccggaggagggtcacaccaacaagctgatcgaacgcaggatcagcgagctggagggacacaagtcgctgtactccgacgccttctattcggccgacgagttcgacgcgctgtacggcggcgagatctaccggaccgtgaagaagacctacgacccagattctcgtttcctcgacctctatgcgaaggcggtgcgacggcaatga

SEQ ID NO: 52 - protein, 463 aa, Mycobacterium gilvum
VSVAVTDARSAYAHGVQRLVASYRAIPAGATVRLAKPTSNLFRARAKSTAAGLDTSGLTHVIAVDPETRTAEVAGMCTYEDLVAATLPHGLSPLVVPQLKTITLGGAVTGLGIESASFRNGLPHESVLEMDILTGTGDIVRAAPDENPDLFRTFPNSYGTLGYSVRLKIELEPVKPFVALRHLRFHSLSTLIATMDRIVDTGSLDGEQVDYLDGVVFSAEESYLCVGTRSATPGPVSDYTGEHIFYRSIQHDCPTEGGQKHDRLTAHDYFWRWDTDWFWCSRAFGAQNPKVRRWWPRRLRRSSFYWKLVGYDQRFGIADRIEKHHGRPPRERVVQDVEVPIERTVEFLQWFLDTIPIEPLWLCPLRLRDDNSWSLYPLRPHRTYVNVGFWSSVPVGPEEGHTNKLIERRISELEGHKSLYSDAFYSADEFDALYGGEIYRTVKKTYDPDSRFLDLYAKAVRRQ

SEQ ID NO: 53 - DNA, 1425 bp, Mycobacterium indicus pranii
atgcacgggctgttgtcgaagactagggtatatgtggtgcctgtccttggatctgcactctcggcccacaagtcgggcgttgaccggctgctggcaagctatcgatccattcccgcaacgtccgcggtccggctggccaaaccgacgtcaaacctgttccgcgcccgcaccaaacgtgacgcgcccggcttggacacctcggggctgaccggcgtcctgagcgtggatcccgaaacccgcaccgcggacgtcgccggcatgtgcacctacgcggacctggtggccgcaacgctgccctacggcctgtcgccgctggtcgtcccgcagctgaagaccatcaccctcggcggggcggtcagcggcctggggatcgagtcggcgtcgtttcgcaacgggctgccgcacgaatcggtgctggagatggatatcctcaccggcgctggcgatttgctcaccgcatcacgtacccagcacccggacctgttccgcgccttcccgaattcctatgggacactggggtattcgacccggcttcggatcgagctggaacccgtcgcaccgttcgtcgcgctgcgccacatccgcttccgctcgctgcccgcgctgatcgccgcggccgaacgcatcgtcgacaccggcgggcagggcggaaccccggtcgactacctcgacggggtggtcttcagcgccgacgaaagctacctgtgcgtgggccggcggaccaccacccccggcccggtcagcgactacaccggcaaggacatctactaccagtccatccggcacgacgccccgggcctggaggcgaccaaggatgaccggctgaccatgcacgactacttctggcgctgggacaccgattggttctggtgctcgcgcgcgttcggcgtgcaggacccgcgggtgcgacgcttctggccgcgccgttatcggcgcagcagcttctactggaagctgatttccctggaccggcgcttcgggatctccgaccgcatcgaggcgcgcaacgggcggcccccacgcgaacgggtggtgcaagacatcgagattccaatcgaacggacctgcgacttcctggagtggttcctggacaacgtgccaatcacgccgatctggttgtgcccgttgcgccttcgcgaccgcgacggctggccgttgtacccgatgcggccggatcacacgtacgtcaacgtcggcttctggtcgtcggtgccggggggcgcgaccgagggcgccgccaaccggatgatcgaagaaaaggtgagcgaactcgacgggcacaagtccctgtactccgattccttctactcccgcgaggacttcgacgagctgtacggcggcgagacctacaacaccgtcaagaaaacctacgaccccgattctcgtttactcgacctctacgcaaaggcggtgcaacggcgatga

SEQ ID NO: 54 - protein, 474 aa, Mycobacterium indicus pranii
MHGLLSKTRVYVVPVLGSALSAHKSGVDRLLASYRSIPATSAVRLAKPTSNLFRARTKRDAPGLDTSGLTGVLSVDPETRTADVAGMCTYADLVAATLPYGLSPLVVPQLKTITLGGAVSGLGIESASFRNGLPHESVLEMDILTGAGDLLTASRTQHPDLFRAFPNSYGTLGYSTRLRIELEPVAPFVALRHIRFRSLPALIAAAERIVDTGGQGGTPVDYLDGVVFSADESYLCVGRRTTTPGPVSDYTGKDIYYQSIRHDAPGLEATKDDRLTMHDYFWRWDTDWFWCSRAFGVQDPRVRRFWPRRYRRSSFYWKLISLDRRFGISDRIEARNGRPPRERVVQDIEIPIERTCDFLEWFLDNVPITPIWLCPLRLRDRDGWPLYPMRPDHTYVNVGFWSSVPGGATEGAANRMIEEKVSELDGHKSLYSDSFYSREDFDELYGGETYNTVKKTYDPDSRLLDLYAKAVQRR

SEQ ID NO: 55 - DNA, 1380 bp, Mycobacterium phlei
gtgtctgaaccccgaaccgacgcacgtgttgttcaggccgcgggcgtgcacaagctgctggagagctaccgcgcgatcccgcccgaggccaccgtccggctggccaaacccacctcgaacctgttccgggcgcgcgccaagacctcggtcaagggtctcgatgtctcgggcctgacccatgtgatctccgtcgaccccgacgagcgcaccgctgaggtggccgggatgtgcacctacgaggacctggtcgccgcgacgctgccgtacgggctgtcaccgctggtggtgccgcagctcaagaccatcaccctcggcggcgccgtgacgggtctgggcatcgagtcggcgtcgttccgtaacggcctgccgcacgagtcggtgctggagatggacatcctcaccggatcgggcgagatcctcaccgcctcccgcgaccagcaccccgacctgttccgggcgttcccgaactcctatggcacgctgggctattcggtgcggctgaagatcgagttggagaccgtcaaaccgttcgtcgcggtccgtcacctgcggttccacgacatcgaggacctggtcgccgagatggaccgcattgtcgagaccggcggctacgacggcaccccggtcgactatctcgacggtgtggtgttctcggcccgcgagagctacctgacgctgggcttccagaccgccaccccgggcccggtcagcgactacaccggccagcagatctactaccgctcgatccagcacgaggacggcgtcaaggacgaccggctgacgatccacgactacttctggcgctgggacaccgactggttctggtgctcgcgggcgttcggcgtgcagaacccgacgatccgccggttctggccgcgccggctcaagcgcagcagcttctactggaagctggtcgcctacgaccgcaagttcaacatcgccgatcgcatcgagatgcacaacggccgcccgccccgcgagcgcgtcgtgcaggacatcgaggtgccgatcgagcgggtcgccgagtttttgggctggttcctcgacaacgtgccgatcgagccgatctggctgtgcccgttgcgtcttcgcgacgacgccggctggccgctgtacccgatccgggcgcagcacacctacgtcaacgtggggttctggtcctcggtgccggtggggcccaccgaggggcacacgaaccggctgatcgagcgcaaggtcagcgagctcgacgggcacaagtcgctgtactcggacgcgtactactcgcgcgacgagttcgaccagctctacggcggcgaaatctacaaaaccgttaaaaaggcctacgatccagattcacgactgctcgacctgtacgcgaaggcggtgcagcgccagtga

SEQ ID NO: 56 - protein, 459 aa, Mycobacterium phlei
VSEPRTDARVVQAAGVHKLLESYRAIPPEATVRLAKPTSNLFRARAKTSVKGLDVSGLTHVISVDPDERTAEVAGMCTYEDLVAATLPYGLSPLVVPQLKTITLGGAVTGLGIESASFRNGLPHESVLEMDILTGSGEILTASRDQHPDLFRAFPNSYGTLGYSVRLKIELETVKPFVAVRHLRFHDIEDLVAEMDRIVETGGYDGTPVDYLDGVVFSARESYLTLGFQTATPGPVSDYTGQQIYYRSIQHEDGVKDDRLTIHDYFWRWDTDWFWCSRAFGVQNPTIRRFWPRRLKRSSFYWKLVAYDRKFNIADRIEMHNGRPPRERVVQDIEVPIERVAEFLGWFLDNVPIEPIWLCPLRLRDDAGWPLYPIRAQHTYVNVGFWSSVPVGPTEGHTNRLIERKVSELDGHKSLYSDAYYSRDEFDQLYGGEIYKTVKKAYDPDSRLLDLYAKAVQRQ

SEQ ID NO: 57 - DNA, 1413 bp, Mycobacterium tuberculosis
atgcaggggcagttgtcgaggactagggtatatacggtgcctgtccctggatctgcacagtcggcttacgcctgcggcgtcgagcggttgctggcgagctatcgatccatccccgcgactgcatccatccggcttgccaagcccacctcaaatctgttccgcgcccgcgtcaaacacgatgcacgcggcctggacgcatcgggactgaccggtgtcatcggtatcgatcccgaggcccgcaccgccgacgtggccggcatgtgcacatacgaggacctaatcgccgcgacactgcactacggtctgtcaccattggtggttccgcagctgaggacgatcacattgggcggagcggtcaccggcttgggtatcgagtcggcgtcgttccgcaacggcctgccccacgagtcggtgctggagatggatatcctcaccggcgcaggagaacttctcaccgtctcgcccggacagcactccgacttgtaccgtgcattccctaactcgtatgggacactgggctattcaacccggcttcgaatccagctggagccggtccggccgtttgtcgcgctgcggcacatccgatttagctcgttgacggcgatggtggccgcaatggagcgcatcatcgacaccggcggactggacggcgaatcggtggactatctcgacggggtggttttcagcgctgacgaaagctacctgtgcatcggcatgcagacgagcgtaccgggcccggtcagcgactacaccggacaagacatctactaccggtcgatccaacacgaggcggggatcaaggaagaccggttgaccatccacgattacttctggcgctgggacaccgattggttctggtgctcacgatcgtttggtgcccaaaacccgcggctgcgccgctggtggccgcggcgctaccggcgtagcagtgtctactggaggttgatggcgctcgatcagcgcttcgggatcgccgaccggttcgagaacagcaggggtcgtcccgcgcgtgaacgggtggtgcaggatatcgaagtgccgatcgaacggacctgcgagtttctggagtggttcggggaaaacgtgcccatttcgccaatctggttgtgcccgttgcggctacgcgatcacgccggctggccgctgtacccgatccggcctgaccgtagctatgtcaacatcgggttctggtcgtcggtgccggttggcgccaccgagggcgccaccaaccgcaagatcgagaacaaggtgagtgcgctcgacgggcacaagtcgctctactccgactccttctatacccgcgaggagttcgacgagctctacggcggcgagacttacaacactgtgaagaaagcctacgatcccgattcgcgtctcctcgatctttacgcaaaggcggtgcaacgacgatga

SEQ ID NO: 58 - protein, 470 aa, Mycobacterium tuberculosis
MQGQLSRTRVYTVPVPGSAQSAYACGVERLLASYRSIPATASIRLAKPTSNLFRARVKHDARGLDASGLTGVIGIDPEARTADVAGMCTYEDLIAATLHYGLSPLVVPQLRTITLGGAVTGLGIESASFRNGLPHESVLEMDILTGAGELLTVSPGQHSDLYRAFPNSYGTLGYSTRLRIQLEPVRPFVALRHIRFSSLTAMVAAMERIIDTGGLDGESVDYLDGVVFSADESYLCIGMQTSVPGPVSDYTGQDIYYRSIQHEAGIKEDRLTIHDYFWRWDTDWFWCSRSFGAQNPRLRRWWPRRYRRSSVYWRLMALDQRFGIADRFENSRGRPARERVVQDIEVPIERTCEFLEWFGENVPISPIWLCPLRLRDHAGWPLYPIRPDRSYVNIGFWSSVPVGATEGATNRKIENKVSALDGHKSLYSDSFYTREEFDELYGGETYNTVKKAYDPDSRLLDLYAKAVQRR

SEQ ID NO: 59 - DNA, 1392 bp, Mycobacterium vanbaalenii; identical to SEQ ID NO: 49

SEQ ID NO: 60 - protein, 463 aa, Mycobacterium vanbaalenii; identical to SEQ ID NO: 50

SEQ ID NO: 61 - DNA, 1452 bp, Rhodococcus opacus
atgcgggagggtggacgccccttccgtgcgcatcgcactctgcccgtcaccgggatcgacgctcaccgcgccggcgtcgaacggcttctcgcgtcctaccgcgcgattcccacggacgccaccgtgcgactcgcgaagaagacgtccaacctgttccgggcgcgggcccagaccagcgcacccggcctcgacgtctccgggctcggcggagtcatctcggtcgacgagcaggaccggaccgcggatgtcgccggaatgtgcacgtacgaagacctggtggacgccaccctcccgtacgggctggcgccgctggtggttccgcaactcaagaccatcacactcggcggcgcggtcaccggcctcggcatcgagtcgacgtcgttccgcaacgggctcccccacgaatcggtcctcgagatcgacgtcctgaccggaagcggcgacatcgtcaccgcgagaccggaaggcgagaactccgacctgttctgggggttccccaactcctacggaaccctcggctactccacccgactgcgcatccagctcgaacccgtcaaacggtatgtggcactgcgccatctgcgtttcgactccctggacgagctgcagtcggcaatggatcgcatcgtcaccgagcgcgtccacgacggcatccccgtcgactatctggacggcgtcgtgttcaccgcgtccgagagttacctgacactgggccatcagaccgacgagggcggccccgtcagcgactacaccgggcagaacatcttctaccggtccatccagcacagttccgtgaaccaccccaaaacggacaaactcaccatccgagactacctgtggcgctgggacaccgactggttctggtgctcgcgcgccttcggcgcccagaaccccaccatccgccggctgtggccgaagaacctcctccgcagcagcttctactggaagctcatcgccctcgaccacaagtacgacatcggcgaccgactcgagaagcgcaagggcaacccgccacgcgaacgcgtcgtgcaggacgtcgaagtgcccatcgagcgcaccgcggacttcgtccgctggttcctcgacgaaatcccgatcgaaccgctgtggctgtgcccgttgcggttgcgggaacctgcccccgccggcgcgtcctcgcaacgcccctggcccctgtaccccctcgaaccgaaacgcacgtacgtgaacatcggattctggtcatcggtgcccatcgttccgggccgacccgagggggccgcgaatcggctgatcgaagacaaggtcagtgacttcgacggacacaagtccctctactccgattcgtactattcacgcgaagatttcgaacgcctctactacggcggcgatcgatacacggaactgaaaaaacgctacgacccgaaatcacgattactggaccttttctccaaggcggtgcaacgtcgatga

SEQ ID NO: 62 - protein, 483 aa, Rhodococcus opacus
MREGGRPFRAHRTLPVTGIDAHRAGVERLLASYRAIPTDATVRLAKKTSNLFRARAQTSAPGLDVSGLGGVISVDEQDRTADVAGMCTYEDLVDATLPYGLAPLVVPQLKTITLGGAVTGLGIESTSFRNGLPHESVLEIDVLTGSGDIVTARPEGENSDLFWGFPNSYGTLGYSTRLRIQLEPVKRYVALRHLRFDSLDELQSAMDRIVTERVHDGIPVDYLDGVVFTASESYLTLGHQTDEGGPVSDYTGQNIFYRSIQHSSVNHPKTDKLTIRDYLWRWDTDWFWCSRAFGAQNPTIRRLWPKNLLRSSFYWKLIALDHKYDIGDRLEKRKGNPPRERVVQDVEVPIERTADFVRWFLDEIPIEPLWLCPLRLREPAPAGASSQRPWPLYPLEPKRTYVNIGFWSSVPIVPGRPEGAANRLIEDKVSDFDGHKSLYSDSYYSREDFERLYYGGDRYTELKKRYDPKSRLLDLFSKAVQRR

SEQ ID NO: 63 - DNA, 1428 bp, Streptomyces regensis
atgatcacactggcaggccgggccggtgcgcgcgatcatgggtgtatggccttcggtgccgccatccccacggggtcgggacacgccgggtacgccgagcgcgtcgcaacccttcgcgcccacctggccgacctcccggaggggacgccggtccggctggcgaagggcacctcgaacctgttccggccgcggtcccgcgccacggcggggctcgacgtgtcggccttcgaccacgtgctgtcgatcgatccgcagaaccggaccgccgacgtcgagggcatggtcacctacgagcggctcgtcgacgcgacgttgccgcacggcctgatgccgctcgtcgttccgcagctcaagacgatcacgctgggcggggcggtcacgggactgggcatcgagtcgtcgtcgttccgcgagggcatgccccacgaatccgtggtggagatggacatcctcacgggtgcgggagacgtggtgaccgcgaccccggacggcgagcacagcgacctgttcttcgggttccccaactcctacggaacgctgggatacgcgctgcgcctgcggatcgaactcgcgccggtgcgcccgtacgtacgactcgaacacctgcgtttctccgatccggcacgctacttcgagcgcctggcgcgtgcgtgccgcgaccgggaggccgacttcgtcgacggcaccgtcttcgctcccgacgagctgtacctgacgttggccacgttcagcggcgagcccgacgaggtcagcgactacacgtggatggacgtctactaccgctcgatcagggagaagacggtcgaccatctgccgatccgcgactacctgtggcggtgggacaccgactggttctggtgttcgcgcgcgctcggagcgcagaaccggctcgtgcggctgctcgcgggtccacgtctgctgcgttccgatgtgtactggaagatcgtcggtttcgaacgcaggcaccggctgtgggagcgtgcgagccggctgctgggcaggcccgagcgcgaagcggtgatgcaggacatcgaggtgccggtgcaccgcgccgaggagttcctgacgttcctgcaccgggagatccccatcagtccggtgtggatctgcccgctgagtgggcgggacgcgcgccggtggccgctgtacgagctcgacccggacgagctgtacgtcaacttcggtttctggggcacggtgccgctcgagccaggcgaaccgcagggttcgcacaaccggcgggtggagaacgtggttaccgaactcgacggacggaaatccctgtactcggagagtttctacgaccgcgacacgttctggcggttgtacggagggaatcaaggacagacgtaccaggccctgaagcatcgctacgacccgaacgggagattgctggacctgtacgccaagtgcgttcaagcgaggtga

SEQ ID NO: 64 - protein, 475 aa, Streptomyces regensis
MITLAGRAGARDHGCMAFGAAIPTGSGHAGYAERVATLRAHLADLPEGTPVRLAKGTSNLFRPRSRATAGLDVSAFDHVLSIDPQNRTADVEGMVTYERLVDATLPHGLMPLVVPQLKTITLGGAVTGLGIESSSFREGMPHESVVEMDILTGAGDVVTATPDGEHSDLFFGFPNSYGTLGYALRLRIELAPVRPYVRLEHLRFSDPARYFERLARACRDREADFVDGTVFAPDELYLTLATFSGEPDEVSDYTWMDVYYRSIREKTVDHLPIRDYLWRWDTDWFWCSRALGAQNRLVRLLAGPRLLRSDVYWKIVGFERRHRLWERASRLLGRPEREAVMQDIEVPVHRAEEFLTFLHREIPISPVWICPLSGRDARRWPLYELDPDELYVNFGFWGTVPLEPGEPQGSHNRRVENVVTELDGRKSLYSESFYDRDTFWRLYGGNQGQTYQALKHRYDPNGRLLDLYAKCVQAR

SEQ ID NO: 65 - DNA, 1413 bp, Thermobifida fusca
gtgaactgtcagtcttccgcgtccaacctcgccaaccacatcaacgcggtgtacgagctgcgccgcgcctatgcgcggctgtccgccgacaagccggtgcgcctggcgaagaccacctccaacctcttccgcttccgcagccgggacgatgccgcgcgtctcgacgtcagcgctttcacctcggtgatcagcatcgacacggaggcgcgggtcgcggaggtgggcggcatgaccacctacgaggacctggtcgccgccaccctgcggcatggcctgatgccgccggtggttccgcaactgcgcacgatcaccctgggcggtgcggtcaccgggctggggatcgaatcctcgtccttccgcaacgggctcccgcacgagtcagtggaagagatggagatcctcaccggcagcggccaggtggtggtggcccggcgcgacaacgagcaccgcgacctgttctacggtttccccaactcgtacggcaccctcggttacgcgctgcggctccgcatccagctcgaaccggtccgcccctacgtccacctgcggcacctgcggttcaccgatgccgcagcggccatggccgcgctggagcagatctgcgcggaccgcacccacgacggggagaccgtcgacttcgtcgacggcgtcgtgttcgcccgcaacgagctgtacctgaccttggggacgttcaccgaccgggctccgtggaccagcgactacaccggaaccgacatctactaccggtcgatcccccgctacgcgggccccggccccggcgactacctcaccacgcacgactacctgtggcggtgggacaccgactggttctggtgctcccgcgccttcggactgcagcatcccgtggtgcgccgcctgtggccgcgttccttgaaacgctccgacgtctaccgcaagctcgtcgcctgggaccggcgcactgacgcgagccgcctgctcgactactaccgcgggcgcccgcccaaggaaccggtgatccaggacatcgaggttgaggtggggcgggctgccgagttcctcgacttcttccacaccgagatcggcatgtccccggtgtggctgtgcccgctgcggctgcgagaagacacagccgacgatacggaaccggtctggccgctctaccccctcaaaccccgccgcctctacgtcaacttcgggttttggggcctcgttccgatccgtcccggtggaggcaggacataccacaaccggctgatcgaaaaagaagtgacccggttgggcgggcacaagtcgctctactcggacgccttctacgacgaggacgagttctgggagctctacaacggggagatctaccgcaagctcaaagctgcctacgaccccgacggtcgactgctcgacctgtacaccaagtgcgtcggcggcgggtga

SEQ ID NO: 66 - protein, 470 aa, Thermobifida fusca
VNCQSSASNLANHINAVYELRRAYARLSADKPVRLAKTTSNLFRFRSRDDAARLDVSAFTSVISIDTEARVAEVGGMTTYEDLVAATLRHGLMPPVVPQLRTITLGGAVTGLGIESSSFRNGLPHESVEEMEILTGSGQVVVARRDNEHRDLFYGFPNSYGTLGYALRLRIQLEPVRPYVHLRHLRFTDAAAAMAALEQICADRTHDGETVDFVDGVVFARNELYLTLGTFTDRAPWTSDYTGTDIYYRSIPRYAGPGPGDYLTTHDYLWRWDTDWFWCSRAFGLQHPVVRRLWPRSLKRSDVYRKLVAWDRRTDASRLLDYYRGRPPKEPVIQDIEVEVGRAAEFLDFFHTEIGMSPVWLCPLRLREDTADDTEPVWPLYPLKPRRLYVNFGFWGLVPIRPGGGRTYHNRLIEKEVTRLGGHKSLYSDAFYDEDEFWELYNGEIYRKLKAAYDPDGRLLDLYTKCVGGG

SEQ ID NO: 67 - DNA, 1368 bp, Thermomonospora curvata
atgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgctga

SEQ ID NO: 68 - protein, 455 aa, Thermomonospora curvata
MSQLAVTDHHERAVEALRRSYAAIPPGTPVRLAKQTSNLFRFREPTAAPGLDVSGFNRVLAVDPDARTADVQGMTTYEDLVDATLPHGLMPLVVPQLKTITLGGAVTGLGIESTSFRNGLPHESVLEMQIITGAGEVVTATPDGEHSDLFWGFPNSYGTLGYALKLKIELEPVKPYVRLRHLRFDDAGECAAKLAELSESREHEGDEVHFLDGTFFGPREMYLTLGTFTDTAPYVSDYTGQHIYYRSIQQRSIDFLTIRDYLWRWDTDWFWCSRALGVQNPLIRRVWPKSAKRSDVYRKLVAYEKRYQFKARIDRWTGKPPREDVIQDIEVPAERLPEFLEFFHDKIGMSPVWLCPLRARHRWPLYPLKPGVTYVNAGFWGTVPLQPGQMPEYHNRLIERKVAQLDGHKSLYSTAFYSREEFWRHYDGETYRRLKDTYDPDARLLDLYDKCVRGR

SEQ ID NO: 69 - DNA, 1452 bp, Rhodococcus triatomae
atgcgggagggccgtcgcccgttccgcacccaggtcgaaccgaaactcgtcggcaccgatgcccaccgccgcggtgtggagcgtctgctcgcctcctaccgtgcgatcccggccgacgccaccgtgcgtctggccaagaagacgtccaacctctttcgcgcccgcgccggtaacgacgctcccggactggacgtctcggggctcaccggcgtcatctcggtcgacacccgggcccgcaccgcagacgtgcagggcatgtgcagctacgaggacctggtggatgccaccctcgcgttcggtctcgctcccacagtggtccctcagctgaagaccatcaccctgggtggggcagtgacgggactgggtatcgaatccacctccttccgcaacgggctgccccacgaggccgtgctcgagatcgacgtgctcaccggcagcggcgagatcctcaccgccacaccggagggcgagcacgcggatctcttctggggcttcccgaactcgtacggaaccctcggctactccacccgcctgcgcatccaactcgagccggtgcgcaggcacgtggccctgcgacatctgcgcttcgactcgctcgaggacctgcaagcggaaatggaccggatcgtcaccgcccgcgagtaccgcggcgagcaggtcgactacctcgacggcgtcgtgttcaccgccaccgagagctacctgacgctggggcggcagaccgacgagtccggcccggtgagcgactacaccggttcgcagatctactaccgctccatccagcacgaatcggtcaatcaccccaagaccgatctgctcaccacccgggactacctgtggcggtgggacaccgactggttctggtgctcccgggcgttcggcgcacagaacccgaaggtgcgcaggctgtggccgaagccgctgctgcgcagcagcttctactggaagctcatcgccctcgatcacaagtacgacgtggccgaccggatcgaggcccgcaagggcaaccccccacgcgagcgcgtcgtccaggacgtcgaggtcccgatcgagcggacggcggacttcctgcactggttcctcaccgagattccgatcgagccgctgtggctgtgcccgttgcgacttcgtgagcctgcgccggccggcgcctcgagccagcggccgtggcccctgtatccgctggaaccgaaacgcacctacgtgaacgtggggttctggtcctcggtgccgagcccgcccgaaaccccggaaggtgccgccaaccggctcatcgaggacaaggtgaacgacttcgacgggcacaagtcgctgtactcggactcgtactactcccgcgaggacttcgagcgcctgtactacggcggcgaccgctataccgagctacgacagaagtacgacccgaaatcccgattactggacctcttctcgaaggcggtgcaacgtcgatga

SEQ ID NO: 70 - protein, 483 aa, Rhodococcus triatomae
MREGRRPFRTQVEPKLVGTDAHRRGVERLLASYRAIPADATVRLAKKTSNLFRARAGNDAPGLDVSGLTGVISVDTRARTADVQGMCSYEDLVDATLAFGLAPTVVPQLKTITLGGAVTGLGIESTSFRNGLPHEAVLEIDVLTGSGEILTATPEGEHADLFWGFPNSYGTLGYSTRLRIQLEPVRRHVALRHLRFDSLEDLQAEMDRIVTAREYRGEQVDYLDGVVFTATESYLTLGRQTDESGPVSDYTGSQIYYRSIQHESVNHPKTDLLTTRDYLWRWDTDWFWCSRAFGAQNPKVRRLWPKPLLRSSFYWKLIALDHKYDVADRIEARKGNPPRERVVQDVEVPIERTADFLHWFLTEIPIEPLWLCPLRLREPAPAGASSQRPWPLYPLEPKRTYVNVGFWSSVPSPPETPEGAANRLIEDKVNDFDGHKSLYSDSYYSREDFERLYYGGDRYTELRQKYDPKSRLLDLFSKAVQRR

SEQ ID NO: 71 - DNA, 1455 bp, Nocardiopsis sp. NRRL B-16309
atgacacctcaccaacagggaacgagcaagcgcgcccccgccggagcgcggtccggcttcgccgagcacatcgcggcggtccagcgtgtgcgccgcgacttccgggcgcttcccgagggcgcccccgtccggctcgccaagcccacgtccaacctcttccgcttccgggagccctcctcggccccctccctcgacgtgtccgccttctccggcgtcatctccatcgacccggtggagcgcctcgccgacgtcggcggcatgaccacctacgaggacctcgtcgccgccacgctcccgcacggactgatgcccaccgtcgtaccgcagctgcgcaccatcaccctgggcggcgcggtcaccgggctcggcatcgagtcgtcctccttccgcagcggcctgccccacgaggcggtccaggagatggagatcctcaccggctcgggcgacgtcgtgaccgccacacgcgacaacgagcacagcgacctcttctacgggttccccaactcctacggcaccctgggctacagcctgcgcctgcgtatcgagctggagccggtctccgcctacgtcaacctccgccacctgcgcttctccgacgcgggcgagtgcatggacgccctcgcgcggatctgcgccgacgccgagcacgacggacagccggtcgacttcgtggacggggtggccttcggccgcgacgagctctacctcaccgtcgcccgcttcaccgaccaggcgccctgggtgagcgactacacgggcaacgacgtctactacaagtcgatcccacgctactccggcaacgggcccggcgactacctcaccacccacgactacctgtggcgctgggacaccgactggttctggtgctcgcgcgccttcggcacgcagcaccccctggtgcggccgctgtggccccgcgcgctcaagcgctcggacgtctaccgcaggctcgtcgcctgggaccggcgcaccgacttcttccggctcctcacccactaccgcggcaagcggccccaggaacccctcatccaggacatcgaggtcggcgtcgagcgcggcgcggagttcctggacttcttccactccgagatcggcatgacgccggtctggatgtgcccgctcaagctcagcgagccgcgtcggccaggccacggcggggacggcgagcacgtctggccgctctaccccctggagaacgaccgcctgtacgtgaacttcggtttctggggtctggtcgacatgctccccggccagcgccgggcccaccacaaccgcagggtggaggaggaggtcgcccggctgggcggccacaagtccctgtactccgacgccttctacaccgaagaggagttctggagcctctacaacggaacggcctacgcgggcctcaaacaggcctacgaccccggggggcgcctcctggatctgtacgccaagtgcgtgggcaaccgctag

SEQ ID NO: 72 - protein, 484 aa, Nocardiopsis sp. NRRL B-16309
MTPHQQGTSKRAPAGARSGFAEHIAAVQRVRRDFRALPEGAPVRLAKPTSNLFRFREPSSAPSLDVSAFSGVISIDPVERLADVGGMTTYEDLVAATLPHGLMPTVVPQLRTITLGGAVTGLGIESSSFRSGLPHEAVQEMEILTGSGDVVTATRDNEHSDLFYGFPNSYGTLGYSLRLRIELEPVSAYVNLRHLRFSDAGECMDALARICADAEHDGQPVDFVDGVAFGRDELYLTVARFTDQAPWVSDYTGNDVYYKSIPRYSGNGPGDYLTTHDYLWRWDTDWFWCSRAFGTQHPLVRPLWPRALKRSDVYRRLVAWDRRTDFFRLLTHYRGKRPQEPLIQDIEVGVERGAEFLDFFHSEIGMTPVWMCPLKLSEPRRPGHGGDGEHVWPLYPLENDRLYVNFGFWGLVDMLPGQRRAHHNRRVEEEVARLGGHKSLYSDAFYTEEEFWSLYNGTAYAGLKQAYDPGGRLLDLYAKCVGNR

SEQ ID NO: 73 - DNA, 1416 bp, Thermobifida cellulosilytica TB100
atgcgtcagccttccgtgtccaacctcgccgaccacatgaaggcggtgcacgaactgcgccgcgcctacacggccctgccggaggacgtgccggtacgcctggccaaaccgacctccaacctcttccgcttcggccgcggcggcaacgcccaccggctcgacgtcagtgccttcacctcggtgatcagcatcgaccccgacgcccggaccgccgaggtcgggggcatgaccacctacgaggacctggtcgccgtcaccctgcggcatgggctcatgccgctggtggtgccgcagttgcgcaccatcaccctgggcggggccgtcaccggcctgggcatcgagtcctcgtccttccgcaacgggctgccgcacgagtcggtggaggagatggagatcctcaccggcaacggcgaggtggtggtggcccggcgcgacaacgagcaccgcgacctgttccgcggcttccccaactcctacggcaccctcggctacgccctgcggctgcgcatccgactcgaaccggtcctcccctacgtccacctccgccacctgcgcttcggcagcgccgaggaggccatggcggccctggagcggatctgcgcggagcagcgctacgaggacgaggcggtcgacttcgtcgacggggtggtcttcgggcgcgacgagctgtacctgacgctggcgtccttcgccgaccgcgccccctggaccagcgactacggcgggtccgacatctactaccggtcgatcccccgctacgcgggacccggccccggcgactacctgaccgtgcacgactacctgtggcggtgggacaccgactggttctggtgctcggccgccttcggggtgcagcacccggtggtgcgccggctgtggccgcgctccctgaagcgctccgacgtctaccgcaggctcgtcgccctggaccggcgcaccgacttcagccgcctgctcgcctactaccgggggcagctccccagggagccggtgatccaggacatcgaggtcgaggtgggccgcggcgcggagttcctcgacttcttccactccgagatcggcatgtccccggtgtggctgtgcccgctgcggctgcgtgaggccccgggcggggagggggacgcggagccggtctggccgctctaccccctcagggccgggcgcctgtatgtcaacttcggcttctggggcatggttcccgtccgccccggaaagggcaggaactaccacaaccgagcggtcgagcaggaggtgacccgcctgggcggccacaagtcgctctactcggacgccttctacagcgaggacgagttctggcggctctacaacggcgaggcctaccgcaagctcaaggccgcctacgacccggaccaccggctgctcgacctgtacgccaagtgcgtcggcaacaggtga

SEQ ID NO: 74 - protein, 471 aa, Thermobifida cellulosilytica TB100
MRQPSVSNLADHMKAVHELRRAYTALPEDVPVRLAKPTSNLFRFGRGGNAHRLDVSAFTSVISIDPDARTAEVGGMTTYEDLVAVTLRHGLMPLVVPQLRTITLGGAVTGLGIESSSFRNGLPHESVEEMEILTGNGEVVVARRDNEHRDLFRGFPNSYGTLGYALRLRIRLEPVLPYVHLRHLRFGSAEEAMAALERICAEQRYEDEAVDFVDGVVFGRDELYLTLASFADRAPWTSDYGGSDIYYRSIPRYAGPGPGDYLTVHDYLWRWDTDWFWCSAAFGVQHPVVRRLWPRSLKRSDVYRRLVALDRRTDFSRLLAYYRGQLPREPVIQDIEVEVGRGAEFLDFFHSEIGMSPVWLCPLRLREAPGGEGDAEPVWPLYPLRAGRLYVNFGFWGMVPVRPGKGRNYHNRAVEQEVTRLGGHKSLYSDAFYSEDEFWRLYNGEAYRKLKAAYDPDHRLLDLYAKCVGNR

SEQ ID NO: 75 - DNA, 1521 bp, Corynebacterium variabile DSM 44702
atgagcctgaaggacgccgcagtcagcatcgccggtagattgaccgatcgcatccacatcggaaatggtcttgaccagcgggaactttccccggtcggatggttcgagcacggacaggccgtccagcgtctcaccgacagcttcgccgccgtgcccgccgataagcgcgtccgcctggcgaagaagacctccaacctcttccgcggccgctccggcgacatggccggcctcgacgtctccggcctgcattccgtcatcgccgtcgacccggtggacaacaccgccgacgtccagggcatgtgcacctacgaggatctggtggacacgctcctgccgttcggcctggtgcccaccgtcgtcccgcagctgaagaccatcaccctcggcggggcggtcaccggcatgggcgtggagtccaccagtttccgcaacggcctgccgcacgaggccgtcctggagatggacgtgctcaccggcaccggcgagatcgtcacctgctcgccgacgcagaacgccgacctctaccgcggtttcccgaactcctacggctcgctgggctactcggtccggctgaagatcacctgcgagaaggtgcccgcctacgtcgaactgcgccacgtccgcttcgacgacgtggagtccgtctccgccgcgctcaccgagatcagcgagtccaaggagtatgacggccagcaggtcgactacctcgacggcgtggtcttctccctcgacgaggcctacctgaccctcggccggcagaccgacgagcccggcccggtgagcgactacacccgcggtggcatctactaccgctcactgcagcacccgacgggcgtctcctacgaccgcctgacgatccgcgactacctctggcgctgggacatcgactggttctggtgcaaccgcgccttcggcacccagaaccccacgatccgcaccctgtggccccgtgacctgctgcgctcgagcttctactggaagatcatcggctgggaccgcaagtacgacctcgccgaccggatcgaggcacacaacggccgcccgccgcgtgagcgcgtcgtccaggacatcgaggtcaccccggcgaacctgccggagtacctcacctggttcttcacccactgcgagatcgagccggtgtggctgtgccccatccggctggccgacggcgtcgaatctctggcaggacgcaccgaggtgctcgacaccgacggtgcggcgacgagtccctggccgctgtacccgctgactccgggcgacacctgggtcaacgtcggcttctggtcctcggtccccgccgacctcatgggtgcggacgccgcccccggtgccttcaaccgggagatcgagcgcgtcgtcgccggtctcggcgggcacaagtcgctgtactccgaggcgtactactcccaggaggagttcgccgccctgtacggcggggatctgccgcagaagctcaaggccgtgtacgacccggacggacgcttccccgggctgtacgagaaaaccgtcaccagcatctag

SEQ ID NO: 76 - protein, 506 aa, Corynebacterium variabile DSM 44702
MSLKDAAVSIAGRLTDRIHIGNGLDQRELSPVGWFEHGQAVQRLTDSFAAVPADKRVRLAKKTSNLFRGRSGDMAGLDVSGLHSVIAVDPVDNTADVQGMCTYEDLVDTLLPFGLVPTVVPQLKTITLGGAVTGMGVESTSFRNGLPHEAVLEMDVLTGTGEIVTCSPTQNADLYRGFPNSYGSLGYSVRLKITCEKVPAYVELRHVRFDDVESVSAALTEISESKEYDGQQVDYLDGVVFSLDEAYLTLGRQTDEPGPVSDYTRGGIYYRSLQHPTGVSYDRLTIRDYLWRWDIDWFWCNRAFGTQNPTIRTLWPRDLLRSSFYWKIIGWDRKYDLADRIEAHNGRPPRERVVQDIEVTPANLPEYLTWFFTHCEIEPVWLCPIRLADGVESLAGRTEVLDTDGAATSPWPLYPLTPGDTWVNVGFWSSVPADLMGADAAPGAFNREIERVVAGLGGHKSLYSEAYYSQEEFAALYGGDLPQKLKAVYDPDGRFPGLYEKTVTSI

SEQ ID NO: 77 - DNA, 1389 bp, Mycobacterium sp. NAZ190054
gtgtctgttgcctcgaccgacgcacgggccgctcacgccgacggtgtgcagcggttgttggccagctaccgggccatccctgccgacgccaccgtccgcctggcaaaacccacctccaacctgttccgggtgcgggccgagaccaccgcgaaggggctggacacctccgggttgacgaacgtgatcgcggtcgacccggacgccggcaccgcggaggtggccggcatgtgcacctacgaaaacctggtcgccgcgacgctgccctacgggctggctcccctggtggtgccacagctcaagaccatcaccctcggcggcgctgtcaccgggctcgggatcgagtcctcgtcgttccgcaacgggttgccccatgaatcggtgctggagatggacatcctgaccggcaccggggacatcgtgtgcgcctcccccgacgagaatcccgagctgtaccgggcgttcccgaactcctatggcacattggggtattcggtccggctgaagatcgagctggagccggtcaaaccgttcgtggcgctgcgccatctccggttccgctccctgtccgccctcgtggaggcgatggaccgagtcgtcgagaccggcggctggaacggcgcacccgtggactacctcgacggtgtggtgttcagcgccgacgagagctacctgtgcctcggtcagcgctccgcgacgccgggtccggtcagcgactacaccggccagaacatctactaccgctccatccagcacgacggcccggacggcgccgagaagcacgaccggctcacggtgcacgactacctgtggcgctgggacaccgactggttctggtgttcaagagctttcggcgcgcagaacccccggatccggcgctggtggccgcggcggtaccggcgcagcagcttctactggaagctgatcggctacgaccagcgctacggcatcgccgaccgcatcgagaaacgccacggccggcccccgcgcgaacgggtggtgcaggacatcgaggtcccgatcgagcggaccgtcgacttcctgcggtggttcctcgacacggtgccgatcgagccgatctggttgtgcccgttgcggctccggggagaccgggactggcccctgtacccgatccgtccacaccacacctacgtcaacgtcggcttctggtcctcggtgccggtcggccccgaagagggctacaccaacaggttgatcgagcggaaggtcagcgagctcgatggacacaaatcgctgtattccgatgcgttctattcgccggaggagtttgattgcctctacggcggagagttgtacaagaccgtgaagaaaacctacgacccagagtcccgtttcctcgatctgtatgcgaaggcggtgcgaaggcaatga

SEQ ID NO: 78 - protein, 462 aa, Mycobacterium sp. NAZ190054
MSVASTDARAAHADGVQRLLASYRAIPADATVRLAKPTSNLFRVRAETTAKGLDTSGLTNVIAVDPDAGTAEVAGMCTYENLVAATLPYGLAPLVVPQLKTITLGGAVTGLGIESSSFRNGLPHESVLEMDILTGTGDIVCASPDENPELYRAFPNSYGTLGYSVRLKIELEPVKPFVALRHLRFRSLSALVEAMDRVVETGGWNGAPVDYLDGVVFSADESYLCLGQRSATPGPVSDYTGQNIYYRSIQHDGPDGAEKHDRLTVHDYLWRWDTDWFWCSRAFGAQNPRIRRWWPRRYRRSSFYWKLIGYDQRYGIADRIEKRHGRPPRERVVQDIEVPIERTVDFLRWFLDTVPIEPIWLCPLRLRGDRDWPLYPIRPHHTYVNVGFWSSVPVGPEEGYTNRLIERKVSELDGHKSLYSDAFYSPEEFDCLYGGELYKTVKKTYDPESRFLDLYAKAVRRQ

SEQ ID NO: 79 - DNA, 1401 bp, Mycobacterium sp. NAZ190054
atgatcggtttactggtcacggaaaggggcgcgatgacacagctggcggtcagagacgaccacgagcgggcggtcgaggcactgcgaaggtcgtatgaggcgatcccgccgggcacgccggtgcgcctggccaaacgtacctccaacctgttccggttccgcgagcccggcagcgcgccggggctggacgtgtccgggttcgacaaggtgctcgcggtcgatcccgagacgcgcaccgccgaggtgcagggcatgaccacctacgaggacctggtcgacgcgaccctgccgcacgggctgatgccgctggtcgtcccgcagctcaagaccatcacgctgggcggggcggtgaccgggctcggcatcgagtccacctcgttccgcaacgggctgccgcacgagtcggtgctggagatgcaggtcatcaccggggccgggcaggtggtcaccgccacccgcgacggcgagcacgccgacctgttctggggtttccccaactcctacgggacgctcggctacacgctgaagctgaagatagagctggagccggtcaaaccgtacgtgcggctgcggcacctgcgcttcgacgacgcgcaggcgtgcgccgccaagctcaaggagctgagcgagagccgctcgcacgaagacgagccggtggacttcctggacggcaccgtgttcggcccgcgcgaggcgtacctgacggtgggcacgttcaccgacaaggccccgtacgtgtcggactacaccgggcagcgcatctactaccagtcgatccagcagcggagcatcgactacctgaccatccgcgactacatctggcgctgggacaccgactggttctggtgctcgggcgcgttcggggtgcagcggccgctggtgcgacggctgtggcccgaccaggccaagcggtcggacgtgtaccgcaggctggtggcctacgagaagcgcttccacttcaaggcgcgcatcgaccggtggacgggcaagccgccgcgcgaggacgtcatccaggacatcgaggtgccggtggaccggctgccggagttcctggagttcttccacgacaaggtcgggatgagcccggtgtggctgtgcccgttgcgggcccgcgagcggtggccgctgtacccgctggagcccgccgccacctacgtcaacgtcggcttctggggcacggtgcccctgcggcccgggcagatgcccgagtaccacaaccgcctgatcgagcggagggtcgccgcgctggacggccacaagtcgctttactcgacggccttctactcgcgcgaggagttctggcgtcactacgacggggaaacctatcggcgtctgaagtcgatgtacgaccccgaagcacgcctgctggatctctacgacaagtgcgtgcggggccgctga

SEQ ID NO: 80 - protein, 466 aa, Actinomadura echinospora
MIGLLVTERGAMTQLAVRDDHERAVEALRRSYEAIPPGTPVRLAKRTSNLFRFREPGSAPGLDVSGFDKVLAVDPETRTAEVQGMTTYEDLVDATLPHGLMPLVVPQLKTITLGGAVTGLGIESTSFRNGLPHESVLEMQVITGAGQVVTATRDGEHADLFWGFPNSYGTLGYTLKLKIELEPVKPYVRLRHLRFDDAQACAAKLKELSESRSHEDEPVDFLDGTVFGPREAYLTVGTFTDKAPYVSDYTGQRIYYQSIQQRSIDYLTIRDYIWRWDTDWFWCSGAFGVQRPLVRRLWPDQAKRSDVYRRLVAYEKRFHFKARIDRWTGKPPREDVIQDIEVPVDRLPEFLEFFHDKVGMSPVWLCPLRARERWPLYPLEPAATYVNVGFWGTVPLRPGQMPEYHNRLIERRVAALDGHKSLYSTAFYSREEFWRHYDGETYRRLKSMYDPEARLLDLYDKCVRGR

SEQ ID NO: 81 - protein, 195 aa, Corynebacterium glyciniphilium
VAVLCTPLLLGACTIGDAGPGDETTDPVVDTEAPPDKPVPDSAAESGAEDGPDSEVPDDPDQPDAEPVETDPDAPGARGLAIGDCVADMDQLDGTGDIDVVDCAGPHAGEVYAQADIAGKNLFPGNEPLGQEAGAICGGDSFTGYVGIGFPESSLDVVTMMPSKESWAQEDRTVTCVVTDPNLEQIAGTLEQSWR

SEQ ID NO: 82 - DNA, 381 bp, Mycobacterium austroafricanum
gtgatccgctttctgctgcgcgtcgcggtctttctcggatcgtcggcgatcgggctactggtggccggctggctggtgccgggggtgtcgctgtcggtgctgggcttcgtcaccgcggtggtgatcttcacggtggcacaagggattctgtcgccgttcttcctgaagatggccagccgctacgcgtcggccttcctcggcggcatcggcctggtgtccacgttcgtggcgctgctgctcgcgtcgctgctgtccaacgggctcagcatccgcggcgtcgggtcgtggatcgcggccacggtggtggtctggctggtcacagccctggcgaccgtcgtgctgcccgttctggtgctgcgggagaagaagaaagcagcctga

SEQ ID NO: 83 - protein, 126 aa, Mycobacterium austroafricanum
VIRFLLRVAVFLGSSAIGLLVAGWLVPGVSLSVLGFVTAVVIFTVAQGILSPFFLKMASRYASAFLGGIGLVSTFVALLLASLLSNGLSIRGVGSWIAATVVVWLVTALATVVLPVLVLREKKKAA

SEQ ID NO: 84 - DNA, 387 bp, Mycobacterium gilvum
atgatccggttcctgctgcgcatcgcggtctttctgggctcatcagcgatcgggctcctcgtcgccggatggctggtgcccggggtgtcgctgtcggtgtggggcttcgtcacggcagtggtgatcttcaccgtggcgcaggcgatcctgtccccgttcttcctcaagatggccagccgctacgcctcggcgttcctcggcgggatcggtctggtgtcgacgtttgccgcgctgctgctcgtctcgctgctgtccaacggtctgagcatccgcggcatcggatcctggatcgccgcaaccgtggtggtctgcttggtgaccgccctggcgacgctggtgctgccgatgttggtgctgcgcgagaagaaaaccgcgtcgcgcgtctga

SEQ ID NO: 85 - protein, 128 aa, Mycobacterium gilvum
MIRFLLRIAVFLGSSAIGLLVAGWLVPGVSLSVWGFVTAVVIFTVAQAILSPFFLKMASRYASAFLGGIGLVSTFAALLLVSLLSNGLSIRGIGSWIAATVVVWLVTALATLVLPMLVLREKKTASRV

SEQ ID NO: 86 - DNA, 381 bp, Mycobacterium vanbaalenii; identical to SEQ ID NO: 82

SEQ ID NO: 87 - protein, 126 aa, Mycobacterium vanbaalenii; identical to SEQ ID NO: 83

SEQ ID NO: 88 - DNA, 501 bp, Streptomyces regensis
gtgcgcgtggcaccgccccgcatcggtgccacacccggcgcggtgggcgcaccggactacgcctccgccttccgcgtgccgacggcggcggcccgcaggcgttcgccgcgggaatggacgcgtgcggtgttcgagggcgcgcccgcgccgttggcgctgttcgtgcgttggggatggctggccgtgctccggttgcgcctcagtgaggaccccgaggcggtggcgggctggagacccacgacgctcgaccccggcacctccgacgcccccgacacctctgagacagccggaaactccgacgctgccgcactggaggccgaatcgccgctgctggaggcgtgcaacgtggcgttcgtcgacgacgacggtgtcacgtgggcgacctacgtccggttccgtggtggcctcggccgcgcggtgtgggcggtggcggcgcggatccaccacgtcgtcatcccctacctgctgcggcgggcggtgcggcgcacggaacgggagtga

SEQ ID NO: 89 - protein, 166 aa, Streptomyces regensis
VRVAPPRIGATPGAVGAPDYASAFRVPTAAARRRSPREWTRAVFEGAPAPLALFVRWGWLAVLRLRLSEDPEAVAGWRPTTLDPGTSDAPDTSETAGNSDAAALEAESPLLEACNVAFVDDDGVTWATYVRFRGGLGRAVWAVAARIHHVVIPYLLRRAVRRTERE

SEQ ID NO: 90 - DNA, 447 bp, Thermobifida fusca
atggctgcgaccgatgacgaccggcaccacaccaccgtcgccctcgacctcatcgacgcgtatgtgcgcgccgaccgcagaatgatcggtgaacgttccgcggggatcagcgcggaggcgggggagcggatcgtctccaccctgaaagtgtgcgcggccttccttgcccgccgggtccaggagaccggggtgccgtggcgcgccgcggactcccgggaagcggtcgcccgcaccgtcgccgacctgctggaacccgaggtggaattcgcggtcgtctccgcctgggaggcgtacgcgatcggggagcacgaggccgcctgggtccgggcgcacggcgatccgctggtcttcgtccacatgctggccgcgttctccgctgctatcggcacagcggtctacggccgtgaggagctgctgcccacgctgcgcagggtgacagcacgataa

SEQ ID NO: 91 - protein, 148 aa, Thermobifida fusca
MAATDDDRHHTTVALIDAYVRADRRMIGLDERSAGISAEAGERIVSTLKVCAAFLARRVQETGVPWRAADSREAVARTVADLLEPEVEFAVVSAWEAYAIGEHEAAWVRAHGDPLVFVHMLAAFSAAIGTAVYGREELLPTLRRVTAR

SEQ ID NO: 92 - DNA, 588 bp, Corynebacterium glyciniphilium
gtggcggtgctgtgcacaccgttgctgctcggagcctgcaccatcggcgacgcgggaccgggggacgagaccacggaccctgtcgtggacactgaagcaccgcccgataaaccggtgccggactctgcggcggaatccggcgctgaagacggacctgattctgaggtgccggacgaccccgaccagcctgatgctgagccggtggagactgatcccgacgccccgggggcccggggactggcgatcggtgactgcgtcgccgacatggaccagctcgacggcaccggcgacatcgacgtcgtcgactgcgccggcccccatgccggcgaggtgtacgcacaggcggatatcgcaggtaagaacctgttccccggcaacgagccgttggggcaggaggcgggagcgatctgcgggggtgactccttcaccggctatgtcggcatcggattccccgagtcctcgctggacgtcgtcacgatgatgccgtccaaggagagctgggcgcaggaggaccggacggtgacctgtgtggtcaccgacccgaacctcgagcagatcgccggcacgctcgagcagagctggcgttag

SEQ ID NO: 93 - DNA, 456 bp, Marinactinospora thermotolerans DSM 45154
atgcctgggaccaatgacgcggacggcgtcacgcagctcgccatctccctcgtcgacgcctatgtgcgcaaggaccgtgacgcggtgggcgcggcggtggccgaagcagagcgctccggcgggatcgacgatgtcacctcggagctgaaggtctacgcctcgttcctcacccgacgggtgcaggagaccggggtggtgtggaagcctgcggactcccgcgaggcggtggcccggacggtcgccgacatcttggagcccgaactggagttcgcggtcgtcaccgcctgggaggcgcactccgtcggcgaacacgaggcggcggagcgtttcacccgtggcgacccgctggttttcctgcacatgctcgccgccttctgcgcggcgatcggccaggcggtctacaagccggccgagctgatctcgacgttgcggatcgcctcggggtcggccgagtag

SEQ ID NO: 94 - protein, 151 aa, Marinactinospora thermotolerans DSM 45154
MPGTNDADGVTQLAISLVDAYVRKDRDAVGAAVAEAERSGGIDDVTSELKVYASFLTRRVQETGVVWKPADSREAVARTVADILEPELEFAVVTAWEAHSVGEHEAAERFTRGDPLVFLHMLAAFCAAIGQAVYKPAELISTLRIASGSAE

SEQ ID NO: 95 - DNA, 492 bp, Nocardiopsis sp. NRRL B-16309
gtgtcctctgacgacaacgcaccccggggcgaccagacacccgcgtccgacctgcccgaccacgtcaaggagaccgcgatcgcgctggtcggcgcctacgccgaccacaaccgcgacgagctcgaccgggtcctgccccgcgccgaggccgattccgccatgctcacctccgaactcaaggtcatcgcggccttcctcagccgcagggtgcagcagacgggcgtggtgtggaagcccgccgactcccgcgaggccgtggcccgcaccgtcgccgagatgctcccgcccgagctggagttcgccgtctccaccgcgtgggaggcacactcggtgggcgaggaggaggtcgccgagcgcttcacccggggcgaccccatggtctacgtgcacatgctcgccgcgttcagcgcggccatcggcctggcggtgtacaagcgggccgaactcgtctccatcctgcgccaggtgatggggctgagcgaaggggactga

SEQ ID NO: 96 - protein, 163 aa, Nocardiopsis sp. NRRL B-16309
MSSDDNAPRGDQTPASDLPDHVKETAIALVGAYADHNRDELDRVLPRAEADSAMLTSELKVIAAFLSRRVQQTGVVWKPADSREAVARTVAEMLPPELEFAVSTAWEAHSVGEEEVAERFTRGDPMVYVHMLAAFSAAIGLAVYKRAELVSILRQVMGLSEGD

SEQ ID NO: 97 - DNA, 441 bp, Nocardiopsis sp. NRRL B-16309
ctgcccgaccacgtcaaggagaccgcgatcgcgctggtcggcgcctacgccgaccacaaccgcgacgagctcgaccgggtcctgccccgcgccgaggccgattccgccatgctcacctccgaactcaaggtcatcgcggccttcctcagccgcagggtgcagcagacgggcgtggtgtggaagcccgccgactcccgcgaggccgtggcccgcaccgtcgccgagatgctcccgcccgagctggagttcgccgtctccaccgcgtgggaggcacactcggtgggcgaggaggaggtcgccgagcgcttcacccggggcgaccccatggtctacgtgcacatgctcgccgcgttcagcgcggccatcggcctggcggtgtacaagcgggccgaactcgtctccatcctgcgccaggtgatggggctgagcgaaggggactga

SEQ ID NO: 98 - protein, 146 aa, Nocardiopsis sp. NRRL B-16309
MPDHVKETAIALVGAYADHNRDELDRVLPRAEADSAMLTSELKVIAAFLSRRVQQTGVVWKPADSREAVARTVAEMLPPELEFAVSTAWEAHSVGEEEVAERFTRGDPMVYVHMLAAFSAAIGLAVYKRAELVSILRQVMGLSEGD

SEQ ID NO: 99 - DNA, 453 bp, Thermobifida cellulosilytica TB100
atggctgcgacagacgacacccggcaccacaaggccgtcgccctcgacctcatcgacgcgtacgcgcacgccgaccgccgactggtccgcgagcgctccgcggggatcagcaccgaggcgggggagcggatcgcctccgaactgaaggtgttcgcggccttcctctcccgccgggtgcaggacaccggggtgccgtggcgtcccgccgacacgcgggaggcggtctcccggaccgtcgcggacctgctggaccccgaggtggagctggcggtcgtcacgacctgggaggcgtacgcggtgggggagcacgaggccgcccgtgtccgggcccagggcgacccgctggtcttcgtgcacatgctggccgcgttctccgcggccgtcgggacggccgtctacggcagggaggagctgctgcccaccctgcgcgcggccgcggagctctccgactga

SEQ ID NO: 100 - protein, 150 aa, Thermobifida cellulosilytica TB100
MAATDDTRHHKAVALDLIDAYAHADRRLVRERSAGISTEAGERIASELKVFAAFLSRRVQDTGVPWRPADTREAVSRTVADLLDPEVELAVVTTWEAYAVGEHEAARVRAQGDPLVFVHMLAAFSAAVGTAVYGREELLPTLRAAAELSD

SEQ ID NO: 101 - DNA, 2664 bp, Thermomonospora curvata
atgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgcgctggtggtgccgagggtggcaatggcggtggcgccatgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtgtga

SEQ ID NO: 102 - DNA, 2664 bp, Thermomonospora curvata
atgacgctggccaaggtcttcgaggagctggtcggggcggacgcccctgtggagctcaccgcctacgacggatcgagagccggacgcctgggcagtgatctgcgggtccacgtgaagtcgccgtacgcggtgtcctacctggtgcactcgccgagcgcgctcgggctggcccgcgcgtacgtggccgggcacctggacgcctacggcgacatgtacacgctgctgcgggagatgacgcagctgaccgaggcgctgacgcccaaggcccggctgcggctgctggccggtgtcctgcaggatccgctgctgcgcgcggcggccagccgccgtctgccgcccccgccgcaggaggtgcggaccggccgcacctcctggttccggcacaccaagcggcgggacgccaaggccatctcccaccactacgacgtgtccaacaccttctatgagtgggtgctgggcccgtcgatgacctacacctgcgcctgtttccccaccgaggacgccaccttggaggaggcgcagttccacaagcacgacctggtcgccaagaagctcgggctgcggccgggcatgcggctgctggacgtgggctgcggctggggcggcatggtgatgcacgccgccaagcactacggggtgcgggcgctgggcgtcacgctgtccaagcagcaggccgagtgggcgcagaaggccatcgccgaggcgggcctgagcgacctggccgaggtccgccaccaggactaccgggacgtcaccgagggcgacttcgacgccatcagctcgatcggcctcaccgagcacatcggcaaggccaacctgccgtcctacttcggcttcctgtacggcaagctcaagccgggcgggcggctgctcaaccactgcatcacccggcccgacaacacccagccggccatgaagaaggacgggttcatcaaccggtacgtcttccccgacggggagctggaggggcccggctacctgcagacccagatgaacgacgccggttttgagatccgccaccaggagaacctgcgcgagcactacgcccgcaccctggccggatggtgccgcaacctcgatgagcactgggacgaggcggtggccgaggtcggcgagggcaccgcgcgggtgtggcggctgtacatggccggcagccggctcggtttcgagctcaactggatccagctgcaccagatcctgggcgtcaagctcggcgagcgcggcgagtcccgcatgccgttgcggcccgactggggcgtggctggtggtgccgagggtggcaatggcggtggcgccatgtcacagctggcggtcacagaccaccacgagcgagcggtcgaggcgctgcgcaggtcgtatgcggcgatcccgccgggcacaccggtccgcttggccaagcagacctccaacctgttccgcttccgcgagccgacggccgcgcccggcctggacgtgtccggcttcaaccgggtgctggcggtggacccggatgcgcgcaccgccgacgtgcagggcatgaccacctacgaggacctggtcgacgccaccctgccgcacgggctgatgccgctggtggtgccccagctcaagacgatcacgctgggcggggcggtgaccggcctgggcatcgagtccacctccttccgcaacggcctgccgcacgagtcggtgctggagatgcagatcatcaccggcgccggcgaagtggtcaccgccaccccggacggggagcactccgacctgttctggggcttccccaactcctacgggacgctggggtacgccctgaagctgaagatcgaactggagccggtcaagccgtacgtccggctgcggcacctgcgcttcgacgacgccggcgagtgcgccgccaagctcgccgagctgagcgaaagccgcgagcacgagggcgatgaggtgcactttttggacggcaccttcttcgggccgcgcgagatgtacctgacgctcggcacgttcaccgacaccgccccctatgtgtcggactacaccgggcagcacatctactaccggtcgatccagcagcggtcgatcgactttttgaccatccgcgactacctgtggcgctgggacaccgactggttctggtgctcgcgcgccctgggcgtgcagaacccgctgatccggcgggtgtggccgaagagcgccaagcggtcggatgtgtaccgcaagctggtggcctacgaaaagcgctaccagttcaaggcgcgcatcgaccggtggacgggcaagccgccgcgcgaggacgtcatccaggacatcgaggtgccggcagaacgcctgccggagttcctggagttcttccacgacaagatcgggatgagcccggtgtggctgtgcccgctgcgggcgcgccaccgctggccgctgtacccgctcaagcccggcgtcacctacgtcaacgccggcttctgggggacggtgccgctgcagccggggcagatgcccgagtaccacaaccggctgatcgaacggaaggtcgcccaactggacggccacaagtctctgtactcgacggcgttctactcgcgtgaggagttctggcggcactacgacggggaaacctaccggcgtctgaaggacacctacgaccccgacgcgcgcctgctcgacctctacgacaagtgcgtgcggggacgctga

SEQ ID NO: 103 - DNA, 1341 bp, Escherichia coli K-12
  FadL (1)..(1341): FadL
atgagccagaaaaccctgtttacaaagtctgctctcgcagtcgcagtggcacttatctccacccaggcctggtcggcaggctttcagttaaacgaattttcttcctctggcctgggccgggcttattcaggggaaggcgcaattgccgatgatgcaggtaacgtcagccgtaaccccgcattgattactatgtttgaccgcccgacattttctgcgggtgcggtttatattgacccggatgtaaatatcagcggaacgtctccatctggtcgtagcctgaaagccgataacatcgcgcctacggcatgggttccgaacatgcactttgttgcaccgattaacgaccaatttggttggggcgcttctattacctctaactatggtctggctacagagtttaacgatacttatgcaggcggctctgtcgggggtacaaccgaccttgaaaccatgaacctgaacttaagcggtgcgtatcgcttaaataatgcatggagctttggtcttggtttcaacgccgtctacgctcgcgcgaaaattgaacgtttcgcaggcgatctggggcagttggttgctggccaaattatgcaatctcctgctggccaaactcagcaagggcaagcattggcagctaccgccaacggtattgacagtaataccaaaatcgctcatctgaacggtaaccagtggggctttggctggaacgccggaatcctgtatgaactggataaaaataaccgctatgcactgacctaccgttctgaagtgaaaattgacttcaaaggtaactacagcagcgatcttaatcgtgcgtttaataactacggtttgccaattcctaccgcgacaggtggcgcaacgcaatcgggttatctgacgctgaacctgcctgaaatgtgggaagtgtcaggttataaccgtgttgatccacagtgggcgattcactatagcctggcttacaccagctggagtcagttccagcagctgaaagcgacctcaaccagtggcgacacgctgttccagaaacatgaaggctttaaagatgcttaccgcatcgcgttgggtaccacttattactacgatgataactggaccttccgtaccggtatcgcctttgatgacagcccagttcctgcacagaatcgttctatctccattccggaccaggaccgtttctggctgagtgcaggtacgacttacgcatttaataaagatgcttcagtcgacgttggtgtttcttatatgcacggtcagagcgtgaaaattaacgaaggcccataccagttcgagtctgaaggtaaagcctggctgttcggtactaactttaactacgcgttctga

SEQ ID NO: 104 - protein, 446 aa, Escherichia coli K-12
  FadL (1)..(446): FadL
MSQKTLFTKSALAVAVALISTQAWSAGFQLNEFSSSGLGRAYSGEGAIADDAGNVSRNPALITMFDRPTFSAGAVYIDPDVNISGTSPSGRSLKADNIAPTAWVPNMHFVAPINDQFGWGASITSNYGLATEFNDTYAGGSVGGTTDLETMNLNLSGAYRLNNAWSFGLGFNAVYARAKIERFAGDLGQLVAGQIMQSPAGQTQQGQALAATANGIDSNTKIAHLNGNQWGFGWNAGILYELDKNNRYALTYRSEVKIDFKGNYSSDLNRAFNNYGLPIPTATGGATQSGYLTLNLPEMWEVSGYNRVDPQWAIHYSLAYTSWSQFQQLKATSTSGDTLFQKHEGFKDAYRIALGTTYYYDDNWTFRTGIAFDDSPVPAQNRSISIPDQDRFWLSAGTTYAFNKDASVDVGVSYMHGQSVKINEGPYQFESEGKAWLFGTNFNYAF

SEQ ID NO: 105 - DNA, 2160 bp, Escherichia coli K-12
  AAS (1)..(2160): AAS
atgctttttagcttttttcgaaatttgtgccgtgttttgtatcgcgttcgcgttacgggtgacacccaggcactgaagggcgagcgcgttctaattacgcctaatcacgtctcttttattgatggcattttgcttggactgtttttacctgtgcgtccagtgtttgccgtttacacctcaataagccaacagtggtatatgcgttggctgaaatcatttatcgactttgttcctctcgacccgacgcaacctatggctattaaacatctggtacgtctggtggaacagggccgaccagtggtgattttccctgaaggacgcatcaccacgacaggctcgctgatgaaaatctacgatggcgcgggttttgtcgcggcgaagtctggtgcaacggttattcctgtgcgtattgaaggggcggaacttacgcacttcagccgcctgaaaggtctggttaaacgtcgcttgttcccgcaaattactctgcatattttgccaccaacgcaggtggcgatgccggatgcgccgcgtgcccgtgaccgtcgcaaaatcgctggcgaaatgctgcatcaaataatgatggaagcgcgaatggcggtgcgcccgcgtgaaacgctgtacgaatctttactgagtgcaatgtaccgcttcggagccgggaagaaatgtgtcgaagacgtcaactttaccccagactcctatcgcaaattgcttacgaaaacgctgtttgttggacgcatccttgaaaaatacagtgttgaaggcgaacgcatcggcttaatgctgcccaatgcaggcatcagtgcggcagtgatttttggggccatcgcccgtcgccgcatgcccgcaatgatgaactacactgccggggtaaaagggctgaccagtgctattacggcggctgaaatcaaaaccatcttcacttcccgccagtttctcgataaaggcaaactctggcatctgccggagcaacttactcaggtgcgctgggtctatctggaagatttaaaagcagatgtcaccactgccgacaaagtatggatcttcgctcatttgctgatgccgcgtctggcacaggttaaacagcagccggaagaagaggcgctgatcctttttacctccggttctgaaggccatccgaaaggcgtcgtccatagccataaaagcattctggcgaatgtcgagcagattaaaacgattgccgacttcaccaccaacgatcgctttatgtcggcgttaccgctgtttcactcctttgggctgacggtaggcctgtttacgccactgcttacaggtgcagaagtgttcctttatccaagcccgctgcattaccgcattgtgccggagttggtgtatgaccgcagttgcaccgtgttgttcggcacctcgactttcctcggtcactacgcgcgtttcgccaacccgtatgacttctatcgtctacgctatgtggtggcaggcgcagaaaaattacaagaaagtaccaaacagctttggcaggataaatttggcctgcgcatccttgaaggctacggcgtgaccgaatgcgcgcctgtcgtttctatcaacgtaccgatggcggcgaaacccggtacggtagggcgtattctaccaggaatggatgcgcgcctgttgtcggtccctggtatcgaagagggcggacgcctgcaactgaaagggccgaacataatgaacggctatctgcgggtggagaagccaggtgtactggaagtgcccaccgccgagaatgttcgcggcgaaatggagcgcggctggtatgacactggcgatattgtgcgttttgacgagcagggctttgtgcagattcagggccgcgcaaaacgctttgccaaaattgcaggcgaaatggtgtcgctggaaatggtggaacaactggcacttggtgtttcgccagataaagtccatgccactgcgattaagagcgatgccagcaaaggcgaggcactggtgcttttcaccacagataacgaactgacgcgcgataagttgcaacagtatgcccgcgagcacggcgtgccggagcttgctgtaccgcgcgatattcgctatctgaaacagatgccattacttggcagcggcaaacctgactttgtcacgttgaaaagctgggtagacgaagcggaacaacacgatgagtga

SEQ ID NO: 106 - protein, 719 aa, Escherichia coli K-12
  AAS (1)..(719): AAS
MLFSFFRNLCRVLYRVRVTGDTQALKGERVLITPNHVSFIDGILLGLFLPVRPVFAVYTSISQQWYMRWLKSFIDFVPLDPTQPMAIKHLVRLVEQGRPVVIFPEGRITTTGSLMKIYDGAGFVAAKSGATVIPVRIEGAELTHFSRLKGLVKRRLFPQITLHILPPTQVAMPDAPRARDRRKIAGEMLHQIMMEARMAVRPRETLYESLLSAMYRFGAGKKCVEDVNFTPDSYRKLLTKTLFVGRILEKYSVEGERIGLMLPNAGISAAVIFGAIARRRMPAMMNYTAGVKGLTSAITAAEIKTIFTSRQFLDKGKLWHLPEQLTQVRWVYLEDLKADVTTADKVWIFAHLLMPRLAQVKQQPEEEALILFTSGSEGHPKGVVHSHKSILANVEQIKTIADFTTNDRFMSALPLFHSFGLTVGLFTPLLTGAEVFLYPSPLHYRIVPELVYDRSCTVLFGTSTFLGHYARFANPYDFYRLRYVVAGAEKLQESTKQLWQDKFGLRILEGYGVTECAPVVSINVPMAAKPGTVGRILPGMDARLLSVPGIEEGGRLQLKGPNIMNGYLRVEKPGVLEVPTAENVRGEMERGWYDTGDIVRFDEQGFVQIQGRAKRFAKIAGEMVSLEMVEQLALGVSPDKVHATAIKSDASKGEALVLFTTDNELTRDKLQQYAREHGVPELAVPRDIRYLKQMPLLGSGKPDFVTLKSWVDEAEQHDE

SEQ ID NO: 107 - DNA, 939 bp, Bacillus subtilis str. SC-8
  nFabH1: 3-oxoacyl-ACP synthase III (1)..(939): nFabH1: 3-oxoacyl-ACP synthase III
atgaaagctggaatacttggtgttggacgttacattcctgagaaggttttaacaaatcatgatcttgaaaaaatggttgaaacttctgacgagtggattcgtacaagaacaggaatagaagaaagaagaatcgcagcagatgatgtgttttcatcacatatggctgttgcagcagcgaaaaatgcgctggaacaagctgaagtggctgctgaggatctggatatgatcttggttgcaactgttacacctgatcagtcattccctacggtctcttgtatgattcaagaacaactcggcgcgaagaaagcgtgtgctatggatatcagcgcggcttgtgcgggcttcatgtacggggttgtaaccggtaaacaatttattgaatccggaacctacaagcatgttctagttgttggtgtagagaagctctcaagcattaccgactgggaagaccgcaatacagccgttctgtttggagacggagcaggcgctgcggtagtcgggccagtcagtgatgacagaggaatcctttcatttgaactaggagccgacggcacaggcggtcagcacttgtatctgaatgaaaaacgacatacaatcatgaatggacgagaagttttcaaatttgcagtccgccaaatgggagaatcatgcgtaaatgtcattgaaaaagccggactttcaaaagaggatgtcgactttttgattccgcatcaggcgaacatccgtatcatggaagctgctcgcgagcgtttagagcttcctgtcgaaaagatgtctaaaactgttcataaatatggaaatacttctgccgcatccattccgatctctcttgtagaagaattggaagccggtaaaatcaaagacggcgatgtggtcgttatggtagggttcggcggaggactaacatggggcgccattgcaatccgctggggccgataa

SEQ ID NO: 108 - protein, 312 aa, Bacillus subtilis str. SC-8
  bFabH1: 3-oxoacyl-ACP synthase III (1)..(312): bFabH1: 3-oxoacyl-ACP synthase III
MKAGILGVGRYIPEKVLTNHDLEKMVETSDEWIRTRTGIEERRIAADDVFSSHMAVAAAKNALEQAEVAAEDLDMILVATVTPDQSFPTVSCMIQEQLGAKKACAMDISAACAGFMYGVVTGKQFIESGTYKHVLVVGVEKLSSITDWEDRNTAVLFGDGAGAAVVGPVSDDRGILSFELGADGTGGQHLYLNEKRHTIMNGREVFKFAVRQMGESCVNVIEKAGLSKEDVDFLIPHQANIRIMEAARERLELPVEKMSKTVHKYGNTSAASIPISLVEELEAGKIKDGDVVVMVGFGGGLTWGAIAIRWGR

SEQ ID NO: 109 - DNA, 1032 bp, Streptomyces coelicolor
  FabH: 3-oxoacyl-ACP synthase III (1)..(1032): FabH: 3-oxoacyl-ACP synthase III
atgtcgaagatcaagcccagcaagggcgccccgtacgcgcgcatcctcggcgtcggcggataccgtccgacccgggtggtgccgaacgaggtgatcctcgagaagatcgactcgtccgacgagtggatccgctcgcgctccggcatcgagacccggcactgggcgggtcccgaggagaccgtcgcggcgatgtcggtggaggcctccggcaaggcactcgccgacgccgggatcgacgcctcgcggatcggtgccgtggtcgtctcgaccgtgtcgcacttcagccagaccccggccatcgccaccgagatcgccgaccgcctcggcacggacaaggccgcggccttcgacatctcggccggctgcgcgggcttcggctacggactgaccctggccaagggcatggtcgtcgaaggttcggcagagtacgtgctggtcatcggcgtggagcggctgtccgacctgaccgacctggaggaccgggccacggccttcctgttcggcgacggcgccggcgcggtcgtggtcggcccgtcccaggagccggcgatcggcccgacggtctggggctcggagggcgacaaggccgaaacgatcaagcagacggtctcctgggaccgcttccggatcggcgacgtctccgaactgcccctcgactccgagggcaacgtcaagtttcctgcgatcacgcaggagggccaggcggtgttccgctgggccgtgttcgagatggcgaaggtcgcgcagcaggcgctggacgcggccgggatcagcccggacgacctggacgtcttcatcccgcaccaggccaatgtgcggatcatcgactcgatggtgaagacactgaagctgccggagcacgtcacggtcgcccgtgacatccgcaccaccggcaacacctcggccgcctcgattccgctcgcgatggagcggctcctggcgaccggcgacgcgaggagcggcgacaccgcgctcgtcatcggcttcggggcgggtctcgtctacgccgcgacggtcgttaccctcccctag

SEQ ID NO: 110 - protein, 343 aa, Streptomyces coelicolor
  FabH: 3-oxoacyl-ACP synthase III (1)..(343): FabH: 3-oxoacyl-ACP synthase III
MSKIKPSKGAPYARILGVGGYRPTRVVPNEVILEKIDSSDEWIRSRSGIETRHWAGPEETVAAMSVEASGKALADAGIDASRIGAVVVSTVSHFSQTPAIATEIADRLGTDKAAAFDISAGCAGFGYGLTLAKGMVVEGSAEYVLVIGVERLSDLTDLEDRATAFLFGDGAGAVVVGPSQEPAIGPTVWGSEGDKAETIKQTVSWDRFRIGDVSELPLDSEGNVKFPAITQEGQAVFRWAVFEMAKVAQQALDAAGISPDDLDVFIPHQANVRIIDSMVKTLKLPEHVTVARDIRTTGNTSAASIPLAMERLLATGDARSGDTALVIGFGAGLVYAATVVTLP

SEQ ID NO: 111 - DNA, 1089 bp, Vibrio cholerae O1 biovar El Tor str. N16961
  FabH2: 3-oxoacyl-ACP synthase III (1)..(1089): FabH2: 3-oxoacyl-ACP synthase III
atgactcaatgttatgccgaaatcaccgggtggggtaaatgtctgccacctgcgacgctctccaatcacgatttgagcacctttcttgatacttccgatgagtggattcaatcacgtactgggattgaacagcgtcggatcagccacgtgaatacttccgatctcgctaccgttgccgctcagcatgccattgcctgtgcgggcgtcagcgttgaagagatagacctgattattgtggcaacctgttcaccggattcgttaattcccaatattgcttcaagagtgcagcaaaatttaggcattccttctgctgccgcctttgatctcaacgccgcctgtaccggtttcctctatggcttagaaaccgcgacacgcttgatgcaagcgagccattatcgtcatgctttggtgattggtgctgagcgcctctctttctatctggactggacgaagcgtgacactgctgtgctgtttggagatggcgcaggtgctgtggtattgagcaagaccgaacaaaaagtgggcttgcaagatgcgcaaattggctgtgatgcccaagggcgagatattttagcggtacctaagtttggaaccgcgatggatcgttttgatgcagacaatggttactgggcgtttgatttcgtgggtaaagagatctttaaacgagcggtacgtggcatgggggctgcggcgcagcaagtgttggcgcgtagcggtttatcgaccgaagaaatcgatgtggtgatcccgcatcaagccaatatccgcattattcaaaccctgtgtgatttggcgggcatcgcgcaagacaaagcctttgtcaatattcatcgctacggtaatacctccgcggctacggtgccgattgccctatgcgaagcgttagagcaaggtaaaatcaaacctcatgatgatctgttggtcgccgcttttggcgcagggctaacgtggggtgcgggtcatattcgctggggtgagcggataacgcctctgggtaaaagcgatgcgcaattgccttcttgtgatcacactgcgttagatctgttgagcaaggctattgagcactgcaagcgtcatcaatcagaatga

SEQ ID NO: 112 - protein, 317 aa, Vibrio cholerae O1 biovar El Tor str. N16961
  FabH2: 3-oxoacyl-ACP synthase III (1)..(317): FabH2: 3-oxoacyl-ACP synthase III
MYTKIIGTGSYLPEQVRTNADLEKMVDTSDEWIVTRTGIRERHIAAPNETVSTMGFEAATRAIEMAGIEKDQIGLIVVATTSATHAFPSAACQIQSMLGIKGCPAFDVAAACAGFTYALSVADQYVKSGAVKYALVVGSDVLARTCDPTDRGTIIIFGDGAGAAVLAASEEPGIISTHLHADGSYGELLTLPNADRVNPENSIHLTMAGNEVFKVAVTELAHIVDETLAANNLDRSQLDWLVPHQANLRIISATAKKLGMSMDNVVVTLDRHGNTSAASVPCALDEAVRDGRIKPGQLVLLEAFGGGFTWGSALVRF

SEQ ID NO: 113 - DNA, 954 bp, Escherichia coli
  eFabH: 3-oxoacyl-ACP synthase III (1)..(954): eFabH: 3-oxoacyl-ACP synthase III
atgtatacgaagattattggtactggcagctatctgcccgaacaagtgcggacaaacgccgatttggaaaaaatggtggacacctctgacgagtggattgtcactcgtaccggtatccgcgaacgccacattgccgcgccaaacgaaaccgtttcaaccatgggctttgaagcggcgacacgcgcaattgagatggcgggcattgagaaagaccagattggcctgatcgttgtggcaacgacttctgctacgcacgctttcccgagcgcagcttgtcagattcaaagcatgttgggcattaaaggttgcccggcatttgacgttgcagcagcctgcgcaggtttcacctatgcattaagcgtagccgatcaatacgtgaaatctggggcggtgaagtatgctctggtcgtcggttccgatgtactggcgcgcacctgcgatccaaccgatcgtgggactattattatttttggcgatggcgcgggcgctgcggtgctggctgcctctgaagagccgggaatcatttccacccatctgcatgccgacggtagttatggtgaattgctgacgctgccaaacgccgaccgcgtgaatccagagaattcaattcatctgacgatggcgggcaacgaagtcttcaaggttgcggtaacggaactggcgcacatcgttgatgagacgctggcggcgaataatcttgaccgttctcaactggactggctggttccgcatcaggctaacctgcgtattatcagtgcaacggcgaaaaaactcggtatgtctatggataatgtcgtggtgacgctggatcgccacggtaatacctctgcggcctctgtcccgtgcgcgctggatgaagctgtacgcgacgggcgcattaagccggggcagttggttctgcttgaagcctttggcggtggattcacctggggctccgcgctggttcgtttctag

SEQ ID NO: 114 - protein, 316 aa, Escherichia coli
  FabD: MalonylCoA:ACP (1)..(316): FabD: MalonylCoA:ACP
MLVLVAPGQGAQTPGFLTDWLALPGAADRVAAWSDAIGLDLAHFGTKADADEIRDTSVAQPLLVAAGILSAAALGTQTSVADATGPGFTPGAVAGHSVGEITAAVFAGVLDDTAALSLVRRRGLAMAEAAAVTETGMSALLGGDPEVSVAHLERLGLTPANVNGAGQIVAAGTMEQLAALNEDKPEGVRKVVPLKVAGAFHTRHMAPAVDKLAEAAKALTPADPKVTYVSNKDGRAVASGTEVLDRLVGQVANPVRWDLCMETFKELGVTAIIEVCPGGTLTGLAKRALPGVKTLALKTPDDLDAARELVAEHTQA

SEQ ID NO: 115 - DNA, 951 bp, Streptomyces coelicolor A3(2)
  FabD: MalonylCoA:ACP (1)..(951): FabD: MalonylCoA:ACP
gtgctcgtactcgtcgctcccggccagggcgcccagacgcccggcttcctgactgactggctcgccctccccggtgccgctgaccgcgtcgccgcgtggtcggacgccatcggactcgatctcgcccacttcggcaccaaggccgacgcggacgagatccgagacacgtccgtggcccagccgctgctggtcgccgccggaatcctgtccgccgcggcactcggtacgcagacatctgtcgctgacgcgacgggccccgggttcacccccggcgcggtcgccggacacagcgtcggcgagatcaccgccgccgtcttcgcgggcgtcctcgacgacaccgccgcgctgtccctcgtacgccgtcgcggcctggccatggccgaggccgcggcggtcaccgagaccggcatgtcggcgctgctcgggggcgaccccgaggtgagcgtcgcgcacctggagcggctcggcctgaccccggcgaacgtgaacggcgccggtcagatcgtggcggcgggcaccatggagcagctggccgcgctgaacgaggacaagcccgagggtgtgcgcaaggtcgtcccgctgaaggtggccggcgcgttccacacccgccacatggcccccgccgtggacaagctcgccgaggccgccaaggcgctgacgccggccgacccgaaggtgacgtacgtctccaacaaggacgggcgggccgtcgcctccggcaccgaggtgctggaccggctggtcggccaggtcgccaacccggtgcgctgggacctgtgcatggagacgttcaaggagctgggcgtcaccgcgatcatcgaggtgtgtccgggcggcacgctgaccgggctggccaagcgggcgctgcccggagtgaagacgctggccctgaagacccccgacgacctcgacgcggcccgtgagctcgtcgccgagcacacccaggcctga

SEQ ID NO: 116 - protein, 316 aa, Streptomyces coelicolor A3(2); identical to SEQ ID NO: 114
  FabD: MalonylCoA:ACP (1)..(316): FabD: MalonylCoA:ACP

SEQ ID NO: 117 - DNA, 768 bp, Streptomyces coelicolor A3(2)
  FabG: 3-oxoacyl-ACP reductase (1)..(768): FabG: 3-oxoacyl-ACP reductase
atgtccgtaaaggaaaacgtgccggagaacagggaactcgcgggaaagcgggccctggtcaccggcggtacgcgcggaatcggagcggcgatcgtccgccggctcctggaggccggcgccgaggtgatcaccaccgccaggtcggcgacgggcccggcgccggagggcgcgggtttcgtgcaggcggacgtgcggaccctcgccggggccgaggcgctcgccggggccgcgctggaggcgctcggcggggtggacatcctggtccacaacgcgggcggggcgcggccccacaaggacggcctggtcatccccgacgaggagtggctggacacgctgaacctgaacctcctcgcgtcggtccggctgaactcccttctggtaccgggcatgcgggagcggcgctcgggggcgatcgtgcacatctcctcggccgcggtcgcgcccccggcgccccccttcctgcactaccaggcggcgaaggcggcgctggacaactacagcgccggactggccgcgacgctggccccctccggcgtccgggtcaacaccgtcagccccggccggaccgccacccccggcggggaggagacccgggagcactgggcgagcctcgacgccggtccggccccgagcggcaccgccccgctgggacgcgacggcctgcccgacgacatcgcccacgcggtgctgtacctcgtgtccgaccgggcgggctgggtgaccgggaccagcctcgccgtggacggcggggaataccccaggggctga

SEQ ID NO: 118 - protein, 255 aa, Streptomyces coelicolor A3(2)
  FabG: 3-oxoacyl-ACP reductase (1)..(255): FabG: 3-oxoacyl-ACP reductase
MSVKENVPENRELAGKRALVTGGTRGIGAAIVRRLLEAGAEVITTARSATGPAPEGAGFVQADVRTLAGAEALAGAALEALGGVDILVHNAGGARPHKDGLVIPDEEWLDTLNLNLLASVRLNSLLVPGMRERRSGAIVHISSAAVAPPAPPFLHYQAAKAALDNYSAGLAATLAPSGVRVNTVSPGRTATPGGEETREHWASLDAGPAPSGTAPLGRDGLPDDIAHAVLYLVSDRAGWVTGTSLAVDGGEYPRG

SEQ ID NO: 119 - DNA, 249 bp, Streptomyces coelicolor A3(2)
  AcpP: Acyl carrier protein (1)..(249): AcpP: Acyl carrier protein
atggccgccactcaggaagagatcgtcgccggtctcgcggagatcgtgaacgagatcgccggcatcccggtcgaggacgtcaagctggacaagtccttcaccgacgacctggacgtcgactcgctgtccatggtcgaggtcgtcgtcgccgccgaagagcgcttcgacgtcaagatcccggacgacgacgtcaagaacctcaagacggtcggcgacgcgacgaagtacatcctcgaccaccaggcctga

SEQ ID NO: 120 - protein, 82 aa, Streptomyces coelicolor A3(2)
  AcpP: Acyl carrier protein (1)..(82): AcpP: Acyl carrier protein
MAATQEEIVAGLAEIVNEIAGIPVEDVKLDKSFTDDLDVDSLSMVEVVVAAEERFDVKIPDDDVKNLKTVGDATKYILDHQA

SEQ ID NO: 121 - DNA, 1290 bp, Streptomyces coelicolor A3(2)
  FabF: 3-oxoacyl-ACP synthase 2 (1)..(1290): FabF: 3-oxoacyl-ACP synthase 2
gtgagctcgaccaatcgcaccgtggtcgtcaccggtatcggcgcaaccacaccgctgggtggcgacgcagcctcgacctgggagggtctggtcgccggcaggtccggcgtccggcccctggagcaggagtgggccgccgaccaggcggtccgtatcgccgcgccggccgccgtcgacccctccgaggtcatcccgcggccgcaggcccgccgcctggaccgctcggcgcagttcgcgctgatcgcggcgcaggaggcctggaaggacgccgggtacgccggcaaggcgggcgagagccccgccgaggacggcgcggcccacgtcgaccccgaccggctcggtgcggtcatcgcctccggcatcggcggcgtgaccacgctgctcgaccagtacgacgtgctgaaggagaagggcgtccgccgcgtctccccgcacaccgtccccatgctgatgccgaacggtccctccgccaacgtcggcctggccgtgggcgcccgcgcgggcgtgcacaccccggtctcggcctgcgcgtcgggcgccgaggccatcggctacgccatcgagatgatccgcaccggccgcgccgacgtcgtcgtcgcgggtggcacggaggcggcgatccacccgctgcccatcgccgcgttcggcaacatgatggcgatgtccaagaacaacgacgacccgcagggcgcctcccgccccttcgacacggcgcgcgacggcttcgtcctcggcgagggcgccggcgtcctggtcctggagtccgccgagcacgccgccgcgcgcggcgcccgcgtctacgcggaggcggtcggccagggcatctccgccgacagccacgacatcgtgcagccggagccggagggccgcggcatctccgccgccctgcagaacctgctggacggcaacgacctggacccggccgagatcgtgcacgtcaacgcgcacgccacctcgacgccggccggtgacatcgccgagctgaaggcgctgcgcaaggtcctcggcgacgacgtcgaccacatggcggtctccggcaccaagtcgatgaccggtcacctgctcggtggcgcgggcggcgtggagtccgtggcgaccgtgctcgcgctgtaccaccgggtggcgccgccgaccatcaacgtcgagaacctcgacccggaggccgaggccaacgcggacatcgtccgcggggaggcccgcaagctcccggtggagggccgtatcgccgcgctgaacgactcgttcggcttcggcgggcacaacgtggtgctcgcgttccggtcggtctga

SEQ ID NO: 122 - protein, 429 aa, Streptomyces coelicolor A3(2)
  FabF: 3-oxoacyl-ACP synthase 2 (1)..(429): FabF: 3-oxoacyl-ACP synthase 2
MSSTNRTVVVTGIGATTPLGGDAASTWEGLVAGRSGVRPLEQEWAADQAVRIAAPAAVDPSEVIPRPQARRLDRSAQFALIAAQEAWKDAGYAGKAGESPAEDGAAHVDPDRLGAVIASGIGGVTTLLDQYDVLKEKGVRRVSPHTVPMLMPNGPSANVGLAVGARAGVHTPVSACASGAEAIGYAIEMIRTGRADVVVAGGTEAAIHPLPIAAFGNMMAMSKNNDDPQGASRPFDTARDGFVLGEGAGVLVLESAEHAAARGARVYAEAVGQGISADSHDIVQPEPEGRGISAALQNLLDGNDLDPAEIVHVNAHATSTPAGDIAELKALRKVLGDDVDHMAVSGTKSMTGHLLGGAGGVESVATVLALYHRVAPPTINVENLDPEAEANADIVRGEARKLPVEGRIAALNDSFGFGGHNVVLAFRSV

SEQ ID NO: 123 - DNA, 1287 bp, Streptomyces coelicolor A3(2)
  FabB2: 3-oxoacyl-ACP synthase (1)..(1287): FabB2: 3-oxoacyl-ACP synthase
gtgagtccacgacacgcggccggcccggcccccggggagcgggccgccgtcgtcacggggctggggctgaccacctcgctgggcggcgacgtcccctccacctggcgggccctgctggacggcgggtgcggcgtcgaacgcgtcgacttcggcgagccggccgggcccgcacaggtgtacctcgcggccccggccgcggtcgaccccgggacggtcctgtcgtccgccaaggcggcgcactgcgaccgctcggcgcagttcgccctcgtggccgcccgcgaggccgttcgcgacgcggggttccccgacccgtccgcgctcgccggggacgggtcgcgggtggccgtggtcgtcggggtggggctgggcggcctcaccagcgtcctggagcaggaccaccgcctgcggacgcagggcgcggggcgggtgagcccgcgcaccatcccggtcatgctgcccaaccacgcggccgccgaagtggggctcatggtgggcgcgaaggccggggtgcacgcgcccgtcagtgcctgcgccgccggtgccgaggccctcgcccaggccctcggcatgatccgggacggccgcgcggacatcgtggtcgcgggcggcaccgaggccgcgctgcatccgctggtcctggccgggttcgcgcggctgcgggccctctcccggcgccacgacgatcccaagggggcctccaggcccttcgacgccgaccgggacggcttcgtgatgggggagggcgccggcatgctcgtggtcgagagcgccgcgcacgccgccgcccgcggcgcccgcgtccacggccggctcaccggggcgggcatcaccaacgacagccaccacgtggcccagccggcgcccggcggtccgggctgcgcggcggccgtcgacgccgcactgcgcgacgcggggctcgtgccggagcagatccagcacgtcaacgcccacgccaccgccacaccgctcggcgacctcggcgaggcgcaggcgctgcacagcgtcttcgccaagggcgtggacgacgtcacggtcagcgccaccaagggggccttcggacacaccctcggtgccgccggagccatcgaggccgtcctcaccgtcctcgccctgcgggacagaacggcaccgccggcctgcagcctcgaacgggtcgaccccgagatcggcctgaacgtcgtcggccgcgacccggcgacactgccctgcgggcccctcgcggccgtcagcacgagcatgggcttcggcgggcacaacgtcgcgctcgccttcgccgacgcctcatga

SEQ ID NO: 124 - protein, 428 aa, Streptomyces coelicolor A3(2)
  FabB2: 3-oxoacyl-ACP synthase (1)..(428): FabB2: 3-oxoacyl-ACP synthase
MSPRHAAGPAPGERAAVVTGLGLTTSLGGDVPSTWRALLDGGCGVERVDFGEPAGPAQVYLAAPAAVDPGTVLSSAKAAHCDRSAQFALVAAREAVRDAGFPDPSALAGDGSRVAVVVGVGLGGLTSVLEQDHRLRTQGAGRVSPRTIPVMLPNHAAAEVGLMVGAKAGVHAPVSACAAGAEALAQALGMIRDGRADIVVAGGTEAALHPLVLAGFARLRALSRRHDDPKGASRPFDADRDGFVMGEGAGMLVVESAAHAAARGARVHGRLTGAGITNDSHHVAQPAPGGPGCAAAVDAALRDAGLVPEQIQHVNAHATATPLGDLGEAQALHSVFAKGVDDVTVSATKGAFGHTLGAAGAIEAVLTVLALRDRTAPPACSLERVDPEIGLNVVGRDPATLPCGPLAAVSTSMGFGGHNVALAFADAS

SEQ ID NO: 125 - DNA, 750 bp, Streptomyces coelicolor A3(2)
  FabG: 3-Ketoacyl-ACP reductase (1)..(750): FabG: 3-Ketoacyl-ACP reductase
atgagcacccccgagagcaaccgcgtcgccctggtcaccggaggttccggcggcatcggccgagtggtggccgagcgcctggcccgggacgggttcgccgtcgccgtccagtacgccggcaaccgggcgaaggccgaggagacggtcgccgccatcaacgccgtcggcggccgggcactcgcggtcggcggcgacgtcgccgacgaggacgcgatgggcaccgccttcgacaccgtacggtcggccttcggcggcatcgacgtggtcgtcaacaccgccggcatcatgatcctcgcgcccgtcgagaccctggacctcgccgacctggaccggatgcaccggaccaacatccgcggcaccttcgtcgtcaaccagcaggccgcccgcaccgtgcgctccggcggagccgtcgtcaacttctccacctccgtggtccgcacccagctgcccacctacggcgcctacggcgcctacgtcgccagcaaggcggcggtcgaggccatgaccctcgtcctcgcccgggaactgcgcggccgggacatcacggtcaacgccgttgcccccggccccacggccactcccctcttcctccagggcaaggacgaggggaccgtcgacaagttcgccaaggcgacccccctggagcgcctcggccgccccgaggacatcgccgagaccgtcgccttcctcgccgggcccgcccgctggatcaacggccaggtcctctacgccaacggcggcctcgcgtaa

SEQ ID NO: 126 - protein, 249 aa, Streptomyces coelicolor A3(2)
  FabG: 3-Ketoacyl-ACP reductase (1)..(249): FabG: 3-Ketoacyl-ACP reductase
MSTPESNRVALVTGGSGGIGRVVAERLARDGFAVAVQYAGNRAKAEETVAAINAVGGRALAVGGDVADEDAMGTAFDTVRSAFGGIDVVVNTAGIMILAPVETLDLADLDRMHRTNIRGTFVVNQQAARTVRSGGAVVNFSTSVVRTQLPTYGAYGAYVASKAAVEAMTLVLARELRGRDITVNAVAPGPTATPLFLQGKDEGTVDKFAKATPLERLGRPEDIAETVAFLAGPARWINGQVLYANGGLA

SEQ ID NO: 127 - DNA, 519 bp, Streptomyces coelicolor A3(2)
  FabA: beta-hydroxyacyl-ACP dehydratase/isomerase (1)..(519): FabA: beta-hydroxyacyl-ACP dehydratase/ isomerase
atggtagataaacgcgaatcctatacaaaagaagaccttcttgcctctggtcgcggtgaactgtttggcgctaaaggcccgcaattgccagcaccgaacatgctgatgatggaccgtgtggtcaaaatgaccgaaacgggtggtaacttcgacaaagggtatgttgaagcagaactggatatcaatccggatctgtggttcttcggatgccactttattggcgatccggttatgccgggatgcctgggcctggacgcaatgtggcagctggtagggttctacctcggctggctgggcggcgaaggtaaaggccgcgcgctgggcgttggcgaagtgaaattcactggtcaggtactgccgacagcgaaaaaagtgacctaccgtattcactttaaacgcattgttaaccgtcgtctgattatgggcctggcggatggcgaagtgctggttgatggtcgtctgatctataccgccagcgacctgaaagtcggtctgttccaggatacgtctgccttctga

SEQ ID NO: 128 - protein, 172 aa, Streptomyces coelicolor A3(2)
  FabA: beta-hydroxyacyl-ACP dehydratase/isomerase (1)..(172): FabA: beta-hydroxyacyl-ACP dehydratase/ isomerase
MVDKRESYTKEDLLASGRGELFGAKGPQLPAPNMLMMDRVVKMTETGGNFDKGYVEAELDINPDLWFFGCHFIGDPVMPGCLGLDAMWQLVGFYLGWLGGEGKGRALGVGEVKFTGQVLPTAKKVTYRIHFKRIVNRRLIMGLADGEVLVDGRLIYTASDLKVGLFQDTSAF

SEQ ID NO: 129 - protein, 341 aa, Synechococcus elongatus (strain PCC 7942)
  long-chain acyl-[acyl-carrier-protein] reductase|EC 1.2.1.80| (1)..(341): long-chain acyl-[acyl-carrier-protein] reductase|EC 1.2.1.80|
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 130 - protein, 556 aa, Drosophila melanogaster
  CP4G1_DROME Cytochrome P450 (1)..(556): CP4G1_DROME Cytochrome P450
MAVEVVQETLQQAASSSSTTVLGFSPMLTTLVGTLVAMALYEYWRRNSREYRMVANIPSPPELPILGQAHVAAGLSNAEILAVGLGYLNKYGETMKAWLGNVLLVFLTNPSDIELILSGHQHLTKAEEYRYFKPWFGDGLLISNGHHWRHHRKMIAPTFHQSILKSFVPTFVDHSKAVVARMGLEAGKSFDVHDYMSQTTVDILLSTAMGVKKLPEGNKSFEYAQAVVDMCDIIHKRQVKLLYRLDSIYKFTKLREKGDRMMNIILGMTSKVVKDRKENFQEESRAIVEEISTPVASTPASKKEGLRDDLDDIDENDVGAKRRLALLDAMVEMAKNPDIEWNEKDIMDEVNTIMFEGHDTTSAGSSFALCMMGIHKDIQAKVFAEQKAIFGDNMLRDCTFADTMEMKYLERVILETLRLYPPVPLIARRLDYDLKLASGPYTVPKGTTVIVLQYCVHRRPDIYPNPTKFDPDNFLPERMANRHYYSFIPFSAGPRSCVGRKYAMLKLKVLLSTIVRNYIVHSTDTEADFKLQADIILKLENGFNVSLEKRQYATVA

SEQ ID NO: 131 - protein, 231 aa, Synechococcus elongatus (strain PCC 7942)
MPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 132 - protein, 386 aa, Arabidopsis thaliana
  Fatty acid hydroxylase (1)..(386): Fatty acid hydroxylase
MEHENCRYHSLHHTQFRTNYSLFMPLYDYIYGTMDESTDTLYEKTLERGDDIVDVVHLTHLTTPESIYHLRIGLASFASYPFAYRWFMRLLWPFTSLSMIFTLFYARLFVAERNSFNKLNLQSWVIPRYNLQYLLKWRKEAINNMIEKAILEADKKGVKVLSLGLMNQGEELNRNGEVYIHNHPDMKVRLVDGSRLAAAVVINSVPKATTSVVMTGNLTKVAYTIASALCQRGVQVSTLRLDEYEKIRSCVPQECRDHLVYLTSEALSSNKVWLVGEGTTREEQEKATKGTLFIPFSQFPLKQLRRDCIYHTTPALIVPKSLVNVHSCENWLPRKAMSATRVAGILHALEGWEMHECGTSLLLSDLDQVWEACLSHGFQPLLLPHH

SEQ ID NO: 133 - DNA, 1161 bp, Arabidopsis thaliana
  AAR protein (1)..(1161): AAR protein
atggagcatgagaattgcagataccactcgctgcaccacacgcagttccggaccaactactccctcttcatgcccttgtatgactacatctacggcacaatggatgaaagcacggatacgttgtacgagaaaactctagaaagaggagatgatatagtggacgtggtgcacttaactcacctgacgacgccagaatccatataccatttgcgcattggcttggcctcatttgcctcctaccccttcgcttatagatggttcatgcgccttttgtggcctttcacctctctctccatgatattcacgctcttctacgcccgcctctttgtcgctgagagaaactccttcaacaagctcaacttgcagtcttgggtgatacctagatataatctacagtacttgttaaaatggaggaaagaagcgatcaataacatgattgagaaagcgatactggaggcagataagaaaggagtgaaggtgcttagtctgggtctcatgaaccaaggggaggagcttaacaggaacggagaggtgtatatccacaaccatccagatatgaaagtgagactggtcgacggcagtagattagcagcagctgttgtgatcaacagtgtacccaaagcaactacaagcgtcgtgatgacaggcaatctcactaaggttgcctacaccatcgcctctgctctctgccagagaggcgttcaggtctccactctgcgcctagacgagtatgagaaaataagatcatgcgttccacaagaatgcagagaccatttggtctatttaacctctgaagcactctcatcaaacaaggtatggctggtgggagaaggaacaacaagagaagagcaggaaaaagccacaaaagggacattgtttataccattctcacagttccccctcaagcagttacgtagagattgtatctatcataccacaccagcattgatagttccaaaatctctggtgaatgtccactcctgtgagaactggttaccgagaaaggcgatgagtgcaactagagtggccggcatattgcacgccttagaaggatgggaaatgcatgagtgtggcacatcccttcttctctcggatttggaccaagtatgggaagcctgtctcagccacggcttccagcctctcctccttccacatcattaa

SEQ ID NO: 134 - DNA, 2976 bp, Arabidopsis thaliana
  CER1 (1)..(2976): CER1
agagttgaagagacacagtgacgacacaaacatattacattcgacggtataatggccacaaaaccaggagtcctcaccgattggccttggacacccctcggaagtttcaagtacatcgtaatagcaccatgggctgtccatagcacatacaggtttgtgacagatgatccagagaagagggatctcgggtacttccttgtgttcccattcttgctcttcagaattctgcacaaccaggtttggatctctctgtcccgttactatacgtcctcgggaaagagacgcatcgtcgacaagggaatcgacttcaatcaggtcgacagggagaccaactgggatgaccaaatattgttcaacggagtgctgttctatataggcatcaacctattgccggaggccaaacaacttccctggtggagaactgacggagtgttgatggcagcgcttattcacaccggaccggtggagttcctctattattggctccacaaagctctccaccatcactttctttattcccgctaccattcccaccaccactcctctatcgtcactgagcccatcacttgtaactattgtttcttaccttgatttttacgctgcctatatttccgtttttctttttataattattatacgtaaaatatacatcgttcacgtcctaactagtagcttttatctgtatataatgggatttgatcaaatgtatgcagcggtgatacatccgtttgcggagcacatagcatacttcatcctcttcgcgataccactacttaccacgttgctaacaaaaacggcgtcaataatttcgttcgccggatacataatctacatagacttcatgaacaacatgggacactgcaacttcgagctaatccctaagcgccttttccacctctttcctcccctcaagttcctctgttacaccccctcgtaagtccttaattaacaactcctcttctgtttcatacactaccaatttggcgtagtaaaagcatttacaagaaaccattcttgatgatctgatacaaatacctagttagatcatattaattaatcctcttatggtcatcataaaatctaagcaaatgataaatcatactaatacaggggatatatgctattataatagaattccatacacgtactccattcctgtataaaataaaggtgacgtgatacagttgtatgcttaatatgatcgtcaactactgaatccctggaccacaacagaaaaaaaaacaattatttaatatatcttacatggttcaacttatcggcacataatccaatttcccacaactttacgcattgatagcatctttaaccaaacatcctttgagaactattttaaatatcaaattcattagtcgatatgtagcaggtggtccctctaaacgctcaagttatattaaaccttctggattcatatttacttttaaatatttgtgactttttttccttcggtattaatttagcgcaatgtgaaagcaaaatttaatagttaaaatacattaaagtttggataataaatggagcatgagaattgcagataccactcgctgcaccacacgcagttccggaccaactactccctcttcatgcccttgtatgactacatctacggcacaatggatgaaagcacggatacgttgtacgagaaaactctagaaagaggagatgatatagtggacgtggtgcacttaactcacctgacgacgccagaatccatataccatttgcgcattggcttggcctcatttgcctcctaccccttcgcttatagatggttcatgcgccttttgtggcctttcacctctctctccatgatattcacgctcttctacgcccgcctctttgtcgctgagagaaactccttcaacaagctcaacttgcagtcttgggtgatacctagatataatctacagtacttgttaaaatggaggaaagaagcgatcaataacatgattgagaaagcgatactggaggcagataagaaaggagtgaaggtgcttagtctgggtctcatgaaccaaggggaggagcttaacaggaacggagaggtgtatatccacaaccatccagatatgaaagtgagactggtcgacggcagtagattagcagcagctgttgtgatcaacagtgtacccaaagcaactacaagcgtcgtgatgacaggcaatctcactaaggttgcctacaccatcgcctctgctctctgccagagaggcgttcaggtctccactctgcgcctagacgagtatgagaaaataagatcatgcgttccacaagaatgcagagaccatttggtctatttaacctctgaagcactctcatcaaacaaggtatggctggtgggagaaggaacaacaagagaagagcaggaaaaagccacaaaagggacattgtttataccattctcacagttccccctcaagcagttacgtagagattgtatctatcataccacaccagcattgatagttccaaaatctctggtgaatgtccactcctgtgagaactggttaccgagaaaggcgatgagtgcaactagagtggccggcatattgcacgccttagaaggatgggaaatgcatgagtgtggcacatcccttcttctctcggatttggaccaagtatgggaagcctgtctcagccacggcttccagcctctcctccttccacatcattaaaactccaaccttggaagattttaggagaatgagagcgacacgctctgtgcttcttttccttatgatccagctcttccacgcacaaatgaactatgaaacatatataaagcgcacacatatatttatgcatatcaagcttttggtgattatggtattgatagagtcaaattaagctcggtgactatggtattaataagagtactatttccttttctctttgtgaactaagaaacatatatatataaagcaaacatgttccagaatatctaagtttttcttctcc

SEQ ID NO: 135 - protein, 422 aa, Jeotgalicoccus sp. ATCC 8456
  terminal olefin-forming fatty acid decarboxylase (1)..(422): terminal olefin-forming fatty acid decarboxylase
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 136 - protein, 2720 aa, Synechococcus sp. (strain ATCC 27264 / PCC 7002 / PR-6)
  Polyketide synthase (1)..(2720): Polyketide synthase
MVGQFANFVDLLQYRAKLQARKTVFSFLADGEAESAALTYGELDQKAQAIAAFLQANQAQGQRALLLYPPGLEFIGAFLGCLYAGVVAVPAYPPRPNKSFDRLHSIIQDAQAKFALTTTELKDKIADRLEALEGTDFHCLATDQVELISGKNWQKPNISGTDLAFLQYTSGSTGDPKGVMVSHHNLIHNSGLINQGFQDTEASMGVSWLPPYHDMGLIGGILQPIYVGATQILMPPVAFLQRPFRWLKAINDYRVSTSGAPNFAYDLCASQITPEQIRELDLSCWRLAFSGAEPIRAVTLENFAKTFATAGFQKSAFYPCYGMAETTLIVSGGNGRAQLPQEIIVSKQGIEANQVRPAQGTETTVTLVGSGEVIGDQIVKIVDPQALTECTVGEIGEVWVKGESVAQGYWQKPDLTQQQFQGNVGAETGFLRTGDLGFLQGGELYITGRLKDLLIIRGRNHYPQDIELTVEVAHPALRQGAGAAVSVDVNGEEQLVIVQEVERKYARKLNVAAVAQAIRGAIAAEHQLQPQAICFIKPGSIPKTSSGKIRRHACKAGFLDGSLAVVGEWQPSHQKEGKGIGTQAVTPSTTTSTNFPLPDQHQQQIEAWLKDNIAHRLGITPQQLDETEPFASYGLDSVQAVQVTADLEDWLGRKLDPTLAYDYPTIRTLAQFLVQGNQALEKIPQVPKIQGKEIAVVGLSCRFPQADNPEAFWELLRNGKDGVRPLKTRWATGEWGGFLEDIDQFEPQFFGISPREAEQMDPQQRLLLEVTWEALERANIPAESLRHSQTGVFVGISNSDYAQLQVRENNPINPYMGTGNAHSIAANRLSYFLDLRGVSLSIDTACSSSLVAVHLACQSLINGESELAIAAGVNLILTPDVTQTFTQAGMMSKTGRCQTFDAEADGYVRGEGCGVVLLKPLAQAERDGDNILAVIHGSAVNQDGRSNGLTAPNGRSQQAVIRQALAQAGITAADLAYLEAHGTGTPLGDPIEINSLKAVLQTAQREQPCVVGSVKTNIGHLEAAAGIAGLIKVILSLEHGMIPQHLHFKQLNPRIDLDGLVTIASKDQPWSGGSQKRFAGVSSFGFGGTNAHVIVGDYAQQKSPLAPPATQDRPWHLLTLSAKNAQALNALQKSYGDYLAQHPSVDPRDLCLSANTGRSPLKERRFFVFKQVADLQQTLNQDFLAQPRLSSPAKIAFLFTGQGSQYYGMGQQLYQTSPVFRQVLDECDRLWQTYSPEAPALTDLLYGNHNPDLVHETVYTQPLLFAVEYAIAQLWLSWGVTPDFCMGHSVGEYVAACLAGVFSLADGMKLITARGKLMHALPSNGSMAAVFADKTVIKPYLSEHLTVGAENGSHLVLSGKTPCLEASIHKLQSQGIKTKPLKVSHAFHSPLMAPMLAEFREIAEQITFHPPRIPLISNVTGGQIEAEIAQADYWVKHVSQPVKFVQSIQTLAQAGVNVYLEIGVKPVLLSMGRHCLAEQEAVWLPSLRPHSEPWPEILTSLGKLYEQGLNIDWQTVEAGDRRRKLILPTYPFQRQRYWFNQGSWQTVETESVNPGPDDLNDWLYQVAWTPLDTLPPAPEPSAKLWLILGDRHDHQPIEAQFKNAQRVYLGQSNHFPTNAPWEVSADALDNLFTHVGSQNLAGILYLCPPGEDPEDLDEIQKQTSGFALQLIQTLYQQKIAVPCWFVTHQSQRVLETDAVTGFAQGGLWGLAQAIALEHPELWGGIIDVDDSLPNFAQICQQRQVQQLAVRHQKLYGAQLKKQPSLPQKNLQIQPQQTYLVTGGLGAIGRKIAQWLAAAGAEKVILVSRRAPAADQQTLPTNAVVYPCDLADAAQVAKLFQTYPHIKGIFHAAGTLADGLLQQQTWQKFQTVAAAKMKGTWHLHRHSQKLDLDFFVLFSSVAGVLGSPGQGNYAAANRGMAAIAQYRQAQGLPALAIHWGPWAEGGMANSLSNQNLAWLPPPQGLTILEKVLGAQGEMGVFKPDWQNLAKQFPEFAKTHYFAAVIPSAEAVPPTASIFDKLINLEASQRADYLLDYLRRSVAQILKLEIEQIQSHDSLLDLGMDSLMIMEAIASLKQDLQLMLYPREIYERPRLDVLTAYLAAEFTKAHDSEAATAAAAIPSQSLSVKTKKQWQKPDHKNPNPIAFILSSPRSGSTLLRVMLAGHPGLYSPPELHLLPFETMGDRHQELGLSHLGEGLQRALMDLENLTPEASQAKVNQWVKANTPIADIYAYLQRQAEQRLLIDKSPSYGSDRHILDHSEILFDQAKYIHLVRHPYAVIESFTRLRMDKLLGAEQQNPYALAESIWRTSNRNILDLGRTVGADRYLQVIYEDLVRDPRKVLTNICDFLGVDFDEALLNPYSGDRLTDGLHQQSMGVGDPNFLQHKTIDPALADKWRSITLPAALQLDTIQLAETFAYDLPQEPQLTPQTQSLPSMVERFVTVRGLETCLCEWGDRHQPLVLLLHGILEQGASWQLIAPQLAAQGYWVVAPDLRGHGKSAHAQSYSMLDFLADVDALAKQLGDRPFTLVGHSMGSIIGAMYAGIRQTQVEKLILVETIVPNDIDDAETGNHLTTHLDYLAAPPQHPIFPSLEVAARRLRQATPQLPKDLSAFLTQRSTKSVEKGVQWRWDAFLRTRAGIEFNGISRRRYLALLKDIQAPITLIYGDQSEFNRPADLQAIQAALPQAQRLTVAGGHNLHFENPQAIAQIVYQQLQTPVPKTQ

SEQ ID NO: 137 - protein, 263 aa, Pseudomonas aeruginosa
  UndA (1)..(263): UndA
GPMIDTFSRTGPLMEAASYPAWTQQLIQDCSESKRRVVEHELYQRMRDNKLSAKVMRQYLIGGWPVVEQFALYMAQNLTKTRFARHPGEDMARRWLMRNIRVELNHADYWVHWSRAHGVTLEDLQAQQVPPELHALSHWCWHTSSADSLIVAIAATNYAIEGATGEWSALVCSNGIYAAAFPEEDRKRAMKWLKMHAQYDDAHPWEALEIIVTLAGLNPTKALQAELRQAICKSYDYMYLFLERCMQQEKTAVTRERLALAEG

SEQ ID NO: 138 - protein, 357 aa, Pseudomonas fluorescens (strain ATCC BAA-477 / NRRL B-23932 / Pf-5)
  fatty acid desaturase (1)..(357): fatty acid desaturase
MQGISASPERMNAQQRAAHVRQVVLARGDELRRRFPLLRHQDALGAGILAFALSGMLGSALLYVTGHLAWWACLLLNAFFASLTHELEHDLIHSMYFRKQRLPHNLMLGLVWLARPSTINPWVRRHLHLNHHKVSGSESDIEERAITNGEPWGIARLLMVGDNMMAAFIRLLRAPGARRKLGILVRTLAVYAPLALLHWGAWYVFLGFHGANGVAALLGSPIQWSQDTASLMHYVDIAVVVIIGPNVLRTFCLHFVSSNMHYYGDIEPGNVIQQTQVLNPWWMWPLQAFCCNFGSTHGIHHFVVREPFYIRQMTASVAHKVMAEMGVRFNDFGTFARANRFTRQEREAMQPAHNARA

SEQ ID NO: 139 - DNA, 1269 bp, Jeotgalicoccus sp. ATCC 8456
  terminal olefin-forming fatty acid decarboxylase (1)..(1269)
atggcaacacttaagagggataagggcttagataatactttgaaagtattaaagcaaggttatctttacacaacaaatcagagaaatcgtctaaacacatcagttttccaaactaaagcactcggtggtaaaccattcgtagttgtgactggtaaggaaggcgctgaaatgttctacaacaatgatgttgttcaacgtgaaggcatgttaccaaaacgtatcgttaatacgctttttggtaaaggtgcaatccatacggtagatggtaaaaaacacgtagacagaaaagcattgttcatgagcttgatgactgaaggtaacttgaattatgtacgagaattaacgcgtacattatggcatgcgaacacacaacgtatggaaagtatggatgaggtaaatatttaccgtgaatctatcgtactacttacaaaagtaggaacacgttgggcaggcgttcaagcaccacctgaagatatcgaaagaatcgcaacagacatggacatcatgatcgattcatttagagcacttggtggtgcctttaaaggttacaaggcatcaaaagaagcacgtcgtcgtgttgaagattggttagaagaacaaattattgagactcgtaaagggaatattcatccaccagaaggtacagcactttacgaatttgcacattgggaagactacttaggtaacccaatggactcaagaacttgtgcgattgacttaatgaacacattccgcccattaatcgcaatcaacagattcgtttcattcggtttacacgcgatgaacgaaaacccaatcacacgtgaaaaaattaaatcagaacctgactatgcatataaattcgctcaagaagttcgtcgttactatccattcgttccattccttccaggtaaagcgaaagtagacatcgacttccaaggcgttacaattcctgcaggtgtaggtcttgcattagatgtttatggtacaacgcatgatgaatcactttgggacgatccaaatgaattccgcccagaaagattcgaaacttgggacggatcaccatttgaccttattccacaaggtggtggagattactggacaaatcaccgttgtgcaggtgaatggatcacagtaatcatcatggaagaaacaatgaaatactttgcagaaaaaataacttatgatgttccagaacaagatttagaagtggacttaaacagtatcccaggatacgttaagagtggctttgtaatcaaaaatgttcgcgaagttgtagacagaacataa

SEQ ID NO: 140 - DNA, 8163 bp, Synechococcus sp. PCC7002
  Ols (1)..(8163): Ols
atggttggtcaatttgcaaatttcgtcgatctgctccagtacagagctaaacttcaggcgcggaaaaccgtgtttagttttctggctgatggcgaagcggaatctgcggccctgacctacggagaattagaccaaaaagcccaggcgatcgccgcttttttgcaagctaaccaggctcaagggcaacgggcattattactttatccaccgggtttagagtttatcggtgcctttttgggatgtttgtatgctggtgttgttgcggtgccagcttacccaccacggccgaataaatcctttgaccgcctccatagcattatccaagatgcccaggcaaaatttgccctcaccacaacagaacttaaagataaaattgccgatcgcctcgaagctttagaaggtacggattttcattgtttggctacagatcaagttgaattaatttcaggaaaaaattggcaaaaaccgaacatttccggcacagatctcgcttttttgcaatacaccagtggctccacgggcgatcctaaaggagtgatggtttcccaccacaatttgatccacaactccggcttgattaaccaaggattccaggatacagaggcgagtatgggcgtttcctggttgccgccctaccatgatatgggcttgatcggtgggattttacagcccatctatgtgggagcaacgcaaattttaatgcctcccgtggcctttttgcagcgaccttttcggtggctaaaggcgatcaacgattatcgggtttccaccagcggtgcgccgaattttgcctatgatctctgtgccagccaaattaccccggaacaaatcagagaactcgatttgagctgttggcgactggctttttccggggccgaaccgatccgcgctgtgaccctcgaaaattttgcgaaaaccttcgctacagcaggctttcaaaaatcagcattttatccctgttatggtatggctgaaaccaccctgatcgtttccggtggtaatggtcgtgcccagcttccccaggaaattatcgtcagcaaacagggcatcgaagcaaaccaagttcgccctgcccaagggacagaaacaacggtgaccttggtcggcagtggtgaagtgattggcgaccaaattgtcaaaattgttgacccccaggctttaacagaatgtaccgtcggtgaaattggcgaagtatgggttaagggcgaaagtgttgcccagggctattggcaaaagccagacctcacccagcaacaattccagggaaacgtcggtgcagaaacgggctttttacgcacgggcgatctgggttttttgcaaggtggcgaactgtatattacgggtcgtttaaaggatctcctgattatccgggggcgcaaccactatccccaggacattgaattaaccgtcgaagtggcccatcccgctttacgacagggggccggagccgctgtatcagtagacgttaacggggaagaacagttagtcattgtccaggaagttgagcgtaaatatgcccgcaaattaaatgtcgcggcagtagcccaagctattcgtggggcgatcgccgccgaacatcaactgcaaccccaggccatttgttttattaaacccggtagcattcccaaaacatccagcgggaagattcgtcgccatgcctgcaaagctggttttctagacggaagcttggctgtggttggggagtggcaacccagccaccaaaaagaaggaaaaggaattgggacacaagccgttaccccttctacgacaacatcaacgaattttcccctgcctgaccagcaccaacagcaaattgaagcctggcttaaggataatattgcccatcgcctcggcattacgccccaacaattagacgaaacggaaccctttgcaagttatgggctggattcagtgcaagcagtacaggtcacagccgacttagaggattggctaggtcgaaaattagaccccactctggcctacgattatccgaccattcgcaccctggctcagtttttggtccagggtaatcaagcgctagagaaaataccacaggtgccgaaaattcagggcaaagaaattgccgtggtgggtctcagttgtcgttttccccaagctgacaaccccgaagctttttgggaattattacgtaatggtaaagatggagttcgcccccttaaaactcgctgggccacgggagaatggggtggttttttagaagatattgaccagtttgagccgcaattttttggcatttccccccgggaagcggaacaaatggatccccagcaacgcttactgttagaagtaacctgggaagccttggaacgggcaaatattccggcagaaagtttacgccattcccaaacgggggtttttgtcggcattagtaatagtgattatgcccagttgcaggtgcgggaaaacaatccgatcaatccctacatggggacgggcaacgcccacagtattgctgcgaatcgtctgtcttatttcctcgatctccggggcgtttctctgagcatcgatacggcctgttcctcttctctggtggcggtacatctggcctgtcaaagtttaatcaacggcgaatcggagttggcgatcgccgccggggtgaatttgattttgacccccgatgtgacccagacttttacccaggcgggcatgatgagtaagacgggccgttgccagacctttgatgccgaggctgatggctatgtgcggggcgaaggttgtggggtcgttctcctcaaacccctggcccaggcagaacgggacggggataatattctcgcggtgatccacggttcggcggtgaatcaagatggacgcagtaacggtttgacggctcccaacgggcgatcgcaacaggccgttattcgccaagccctggcccaagccggcattaccgccgccgatttagcttacctagaggcccacggcaccggcacgcccctgggtgatcccattgaaattaattccctgaaggcggttttacaaacggcgcagcgggaacagccctgtgtggtgggttctgtgaaaacaaacattggtcacctcgaggcagcggcgggcatcgcgggcttaatcaaggtgattttgtccctagagcatggaatgattccccaacatttgcattttaagcagctcaatccccgcattgatctagacggtttagtgaccattgcgagcaaagatcagccttggtcaggcgggtcacaaaaacggtttgctggggtaagttcctttgggtttggtggcaccaatgcccacgtgattgtcggggactatgctcaacaaaaatctccccttgctcctccggctacccaagaccgcccttggcatttgctgaccctttctgctaaaaatgcccaggccttaaatgccctgcaaaaaagctatggagactatctggcccaacatcccagcgttgacccacgcgatctctgtttgtctgccaataccgggcgatcgcccctcaaagaacgtcgtttttttgtctttaaacaagtcgccgatttacaacaaactctcaatcaagattttctggcccaaccacgcctcagttcccccgcaaaaattgcctttttgtttacggggcaaggttcccaatactacggcatggggcaacaactgtaccaaaccagcccagtatttcggcaagtgctggatgagtgcgatcgcctctggcagacctattcccccgaagcccctgccctcaccgacctgctgtacggtaaccataaccctgacctcgtccacgaaactgtctatacccagcccctcctctttgctgttgaatatgcgatcgcccaactatggttaagctggggcgtgacgccagacttttgcatgggccatagcgtcggcgaatatgtcgcggcttgtctggcgggggtattttccctggcagacggcatgaaattaattacggcccggggcaaactgatgcacgccctacccagcaatggcagtatggcggcggtctttgccgataaaacggtcatcaaaccctacctatcggagcatttgaccgtcggagccgaaaacggttcccatttggtgctatcaggaaagaccccctgcctcgaagccagtattcacaaactccaaagccaagggatcaaaaccaaacccctcaaggtttcccatgctttccactcccctttgatggctcccatgctggcagagtttcgggaaattgctgaacaaattactttccacccgccgcgtatcccgctcatttccaatgtcacgggcggccagattgaagcggaaattgcccaggccgactattgggttaagcacgtttcgcaacccgtcaaatttgtccagagcatccaaaccctggcccaagcgggtgtcaatgtttatctcgaaatcggcgtaaaaccagtgctcctgagtatgggacgccattgcttagctgaacaagaagcggtttggttgcccagtttacgtccccatagtgagccttggccggaaattttgaccagtctcggcaaactgtatgagcaagggctaaacattgactggcagaccgtggaagctggcgatcgccgccggaaactgattctgcccacctatcccttccaacggcaacgatattggtttaatcaaggctcttggcaaactgttgagaccgaatctgtgaacccaggccctgacgatctcaatgattggttgtatcaggtggcgtggacgcccctggacactttgcccccggcccctgaaccgtcggctaagctgtggttaatcttgggcgatcgccatgatcaccagcccattgaagcccaatttaaaaacgcccagcgggtgtatctcggccaaagcaatcattttccgacgaatgccccctgggaagtatctgccgatgcgttggataatttatttactcacgtcggctcccaaaatttagcaggcatcctttacctgtgtcccccaggggaagacccagaagacctagatgaaattcaaaagcaaaccagtggcttcgccctccaactgatccaaaccctgtatcaacaaaagatcgcggttccctgctggtttgtgacccaccagagccaacgggtgcttgaaaccgatgctgtcaccggatttgcccaagggggattatggggactcgcccaggcgatcgccctcgaacatccagagttgtgggggggaattattgatgtcgatgacagcctgccaaattttgcccagatttgccaacaaagacaggtgcagcagttggccgtgcggcaccaaaaactctacggggcacagctcaaaaagcaaccgtcactgccccagaaaaatctccagattcaaccccaacagacctatctagtgacagggggactgggggccattggccgtaaaattgcccaatggctagccgcagcaggagcagaaaaagtaattctcgtcagccggcgcgctccggcagcggatcagcagacgttaccgaccaatgcggtggtttatccttgcgatttagccgacgcagcccaggtggcaaagctgtttcaaacctatccccacatcaaaggaattttccatgcggcgggtaccttagctgatggtttgctgcaacaacaaacttggcaaaagttccagaccgtcgccgccgccaaaatgaaagggacatggcatctgcaccgccatagtcaaaagctcgatctggatttttttgtgttgttttcctctgtggcaggggtgctcggttcaccgggacaggggaattatgccgccgcaaaccggggcatggcggcgatcgcccaatatcgacaagcccaaggtttacccgccctggcgatccattgggggccttgggccgaagggggaatggccaactccctcagcaaccaaaatttagcgtggctgccgcccccccagggactaacaatcctcgaaaaagtcttgggcgcccagggggaaatgggggtctttaaaccggactggcaaaacctggccaaacagttccccgaatttgccaaaacccattactttgcagccgttattccctctgctgaggctgtgcccccaacggcttcaatttttgacaaattaatcaacctagaagcttctcagcgggctgactatctactggattatctgcggcggtctgtggcgcaaatcctcaagttagaaattgagcaaattcaaagccacgatagcctgttggatctgggcatggattcgttgatgatcatggaggcgatcgccagcctcaagcaggatttacaactgatgttgtaccccagggaaatctacgaacggcccagacttgatgtgttgacggcctatctagcggcggaattcaccaaggcccatgattctgaagcagcaacggcggcagcagcgattccctcccaaagcctttcggtcaaaacaaaaaaacagtggcaaaaacctgaccacaaaaacccgaatcccattgcctttatcctctctagcccccggtcgggttcgacgttgctgcgggtgatgttagccggacatccggggttatattcgccgccagagctgcatttgctcccctttgagactatgggcgatcgccaccaggaattgggtctatcccacctcggcgaagggttacaacgggccttaatggatctagaaaacctcaccccagaggcaagccaggcgaaggtcaaccaatgggtcaaagcgaatacacccattgcagacatctatgcctatctccaacggcaggcggaacaacgtttactcatcgacaaatctcccagctacggcagcgatcgccatattctagaccacagcgaaatcctctttgaccaggccaaatatatccatctggtacgccatccctacgcggtgattgaatcctttacccgactgcggatggataaactgctgggggccgagcagcagaacccctacgccctcgcggagtccatttggcgcaccagcaaccgcaatattttagacctgggtcgcacggttggtgcggatcgatatctccaggtgatttacgaagatctcgtccgtgacccccgcaaagttttgacaaatatttgtgatttcctgggggtggactttgacgaagcgctcctcaatccctacagcggcgatcgccttaccgatggcctccaccaacagtccatgggcgtcggggatcccaatttcctccagcacaaaaccattgatccggccctcgccgacaaatggcgctcaattaccctgcccgctgctctccagctggatacgatccagttggccgaaacgtttgcttacgatctcccccaggaaccccagctaacaccccagacccaatccttgccctcgatggtggagcggttcgtgacagtgcgcggtttagaaacctgtctctgtgagtggggcgatcgccaccaaccattggtgctacttctccacggcatcctcgaacagggggcctcctggcaactcatcgcgccccagttggcggcccagggctattgggttgtggccccagacctgcgtggtcacggcaaatccgcccatgcccagtcctacagcatgcttgattttttggctgacgtagatgcccttgccaaacaattaggcgatcgcccctttaccttggtgggccactccatgggttccatcatcggtgccatgtatgcaggaattcgccaaacccaggtagaaaagttgatcctcgttgaaaccattgtccccaacgacatcgacgacgctgaaaccggtaatcacctgacgacccatctcgattacctcgccgcgcccccccaacacccgatcttccccagcctagaagtggccgcccgtcgcctccgccaagccacgccccaactacccaaagacctctcggcgttcctcacccagcgcagcaccaaatccgtcgaaaaaggggtgcagtggcgttgggatgctttcctccgtacccgggcgggcattgaattcaatggcattagcagacgacgttacctggccctgctcaaagatatccaagcgccgatcaccctcatctatggcgatcagagtgaatttaaccgccctgctgatctccaggcgatccaagcggctctcccccaggcccaacgtttaacggttgctggcggccataacctccattttgagaatccccaggcgatcgcccaaattgtttatcaacaactccagacccctgtacccaaaacacaataa

SEQ ID NO: 141 - DNA, 1074 bp, Pseudomonas protegens Pf-5
  FA-desaturase AAY95614.1 (1)..(1074): FA-desaturase AAY95614.1
atgcagggtatttctgcaagccccgagcggatgaacgcacagcagcgggcggcgcatgtacgccaggtggtcctggcccggggtgatgaactgcgccggcgttttcccttgctgcggcaccaggacgcgctgggcgcggggatccttgcgttcgctctgtcggggatgctggggtcagcgctgctctatgtcacgggtcacctggcctggtgggcgtgcctgctgctcaacgcgttcttcgcctccctgacccacgagctggaacacgacctgatccacagcatgtactttcgcaagcagcgcctgccccacaacctgatgctggggctggtgtggctggcgcggccgagcaccatcaacccctgggtgcgccgccacctgcacctgaatcatcacaaggtgtccggcagcgagagcgacatcgaggagcgcgccatcaccaacggcgagccctggggcatcgcccgcctgctgatggtgggggacaacatgatggcggccttcatccgcctgctgcgggcgccgggggccaggcgcaagctgggcatcctcgttcgtaccctggcggtgtacgcgccgctggcattgctgcattggggcgcctggtatgtgttcctgggctttcatggggccaacggcgtggccgccctgctgggcagcccgatccagtggtcgcaagacaccgcgtcgctgatgcactacgtcgatatcgccgtggtggtgatcatcggccccaacgtgctgcgcaccttctgcctgcactttgtcagttccaacatgcactactacggcgacatcgagccgggcaacgtgatccagcagacccaggtactcaacccctggtggatgtggccgttgcaggccttctgctgcaacttcggcagcacccacggcatccaccatttcgtggtcagggagccgttctacatccgccagatgacggcatcggtggcgcataaagtgatggccgagatgggcgtgcgcttcaacgatttcggcacttttgcccgggccaaccgctttacccggcaggagcgcgaagcgatgcaaccggcgcataacgcccgggcttga

(19) United States
(12) Patent Application Publication
(10) Pub. No.: US 2021/0230653 A1
(43) Pub. Date: Jul. 29, 2021

(54) PROCESSES FOR PRODUCTION OF ALKYLATED FATTY ACIDS AND DERIVATIVES THEREOF

(71) Applicant: ExxonMobil Research and Engineering Company, Annandale, NJ (US)

(72) Inventors: Christopher M. Flynn, Bridgewater, NJ (US); Mark P. Hagemeister, Morris Plains, NJ (US)

(21) Appl. No.: 17/142,771

(22) Filed: Jan. 6, 2021

Related U.S. Application Data
(60) Provisional application No. 62/966,647, filed on Jan. 28, 2020.

(51) Int. Cl. C12P 7/64; C12N 9/10; C12N 9/06; C07C 69/34; C10M 105/34

(52) U.S. Cl. CPC C12P 7/6436; C12Y 203/01041; C12N 9/1029; C10M 2207/281; C12N 9/0026; C07C 69/34; C10M 105/34; C12N 9/1007

(57) ABSTRACT

The present disclosure provides processes for producing alkylated fatty acids and derivatives thereof. In at least one embodiment, a process includes introducing a terminal alkyl transferase and a fatty acid into a bioreactor. The process includes introducing an internal methyl transferase and internal methyl reductase into the bioreactor or a second bioreactor. The process includes obtaining an alkylated fatty acid having a methyl substituent located at an internal carbon atom of the fatty acid and a methyl substituent or ethyl substituent located at a carbon atom alpha to the terminal carbon atom of the fatty acid.

### CROSS-REFERENCE TO RELATED APPLICATIONS

This application is related to and claims the benefit of priority from U.S. Provisional Application No. 62/966,647 filed Jan. 28, 2020, which is hereby incorporated by reference in its entirety.

### REFERENCE TO A SEQUENCE LISTING

This application contains references to amino acid and/or nucleic acid sequences which have been submitted as the sequence listing text file entitled “2020EM028-US2-SequenceListing.txt”, file size 381 KiloBytes (KB), created 5 Jan. 2021, which is hereby incorporated by reference in its entirety.

### FIELD OF THE INVENTION

The present disclosure provides processes for producing alkylated fatty acids and derivatives thereof.

### BACKGROUND OF THE INVENTION

There is increasing concern regarding sustainability within the chemical industry, and there is a growing demand for chemicals produced from renewable resources. In fact, many chemical companies and their customers have implemented sustainability initiatives with a goal of reducing the use of current chemicals, such as petro-based chemicals, with chemicals made from renewable sources. Such companies are seeking renewable chemicals that have minimal impact on product performance or characteristics, as well as minimal impact on downstream products and customers.

Fatty acids derived from agricultural plant and animal oils find use as industrial lubricants, hydraulic fluids, greases, and other specialty fluids in addition to oleochemical feedstocks for processing. The physical and chemical properties of these fatty acids result in large part from their carbon chain length and number of unsaturated double bonds. Fatty acids are typically 12:1 (12 carbons, 1 double bond), 14:1 (14 carbons, 1 double bond), 16:0 (sixteen carbons, zero double bonds), 16:1 (sixteen carbons, 1 double bond), 18:0, 18:1, 18:2, or 18:3. Importantly, fatty acids with no double bonds (saturated) have high oxidative stability, but they solidify at low temperature. Double bonds improve low-temperature fluidity, but decrease oxidative stability. This trade-off poses challenges for lubricant and other specialty-fluid formulations because consistent long-term performance (high oxidative stability) over a wide range of operating temperatures can be desirable.

High 18:1 (oleic) fatty acid oils provide low temperature fluidity with relatively good oxidative stability. Accordingly, several commercial products, such as high oleic soybean oil, high oleic sunflower oil, and high oleic algal oil have been developed. However, oleic acid is an alkene and thus still subject to oxidative degradation.

Whereas straight-chain fatty acids are relatively abundant, non-straight-chain fatty acids are not. Important classes of non-straight-chain fatty acids include branched-chain fatty acids, furan-containing fatty acids, and cyclic fatty acids. The current market for fatty acids includes only linear fatty acids, which have low cetane number and tend to solidify at low temperatures (i.e., it is “waxy”). Methylation at the terminus of the linear fatty acids has been attempted, but the improvements would be limited because methylation at the terminus does not significantly assist in breaking up the waxy properties promoted by the linear backbone of the fatty acid.

There is a need for processes to form fatty acids and derivatives thereof that alleviate the barriers to market caused by the poor cold-flow, low oxidative stability, and cetane characteristics of linear bio-products.

### REFERENCES FOR BACKGROUND INCLUDE

Chiou-Yan Lai, et al., “β-Ketoacyl-Acyl Carrier Protein Synthase III (FabH) Is Essential for Bacterial Fatty Acid Synthesis”, J. Bio. Chem., Vol. 278 (51), pp. 51494-51503 (2003).
U.S. Pub. No. 2012/0164713; U.S. Pat. No. 9,809,804; U.S. Pub. No. 2018/0119045; U.S. Pub. No. 2015/0376659; U.S. Pub. No. 2018/0105848; U.S. Pub. No. 2018/0171252; U.S. Pat. No. 10,113,208; EP3317419; and EP2446041.
Sanjay B. Hari, et al., “Structural and Functional Analysis of E. coli Cyclopropane Fatty Acid Synthase”, Structure, Vol. 26, pp. 1251-1258 (2018).
Shuntaro Machida, et al., “Expression of Genes for a Flavin Adenine Dinucleotide-Binding Oxidoreductase and a Methyltransferase from Mycobacterium chlorophenolicum Is Necessary for Biosynthesis of 10-Methyl Stearic Acid from Oleic Acid in Escherichia coli”, Frontiers in Microbio., Vol. 8, Article 2061, (2017).
Keum-Hwa Choi, et al., “β-Ketoacyl Carrier Protein Synthase III (FabH) Is a Determining Factor in Branched-Chain Fatty Acid Biosynthesis”, J. of Bacteriology, pp. 365-370, (2000).
Current Opinion in Chemical Biology; Volume 35, December 2016, Pages 22-28;
Appl. Environ. Microbiol. 2011 March; 77(5): 1718-1727.
Metabolic Engineering Communications; Volume 7, December 2018, e00076.
APPLIED AND ENVIRONMENTAL MICROBIOLOGY, June 2011, p. 4264-4267;
ACS Catal., 2015, 5, 12, 7091-7094.
The publication available at https://pubs.acs.org/doi/suppl/10.1021/acscatal.5b01842/suppl_file/cs5b01842_si_001.pdf.
Science 30 Jul. 2010: Vol. 329, Issue 5991, pp. 559-562.
Proc. Natl. Acad. Sci. USA. 2012 Sep. 11; 109(37):14858-63.
The Plant Cell, Vol. 7, 2115-2127, December 1995.

### SUMMARY OF THE INVENTION

The present disclosure provides processes for producing alkylated fatty acids and derivatives thereof.

In at least one embodiment, a process includes introducing a terminal alkyl transferase and a fatty acid into a bioreactor. The process includes introducing an internal methyl transferase and internal methyl reductase into the bioreactor or a second bioreactor (other than the bioreactor for the terminal alkyl transferase). The process includes obtaining an alkylated fatty acid having a methyl substituent located at an internal carbon atom of the fatty acid and a terminal methyl substituent or terminal ethyl substituent located at a carbon atom alpha to the terminal carbon atom of the fatty acid.

In at least one embodiment, a fatty acid ester has a methyl substituent. The fatty acid ester has (1) an ethyl substituent or (2) an additional methyl substituent, where the ethyl substituent or the additional methyl substituent is located at a carbon atom alpha to the terminal carbon atom of the fatty acid. The fatty acid ester optionally has an alcohol substituent.

In at least one embodiment, a lubricant includes a fatty acid ester.

### DETAILED DESCRIPTION OF THE INVENTION

The present disclosure provides processes for producing alkylated fatty acids and derivatives thereof.

In at least one embodiment, a process includes introducing a terminal alkyl transferase and an unsaturated fatty acid into a bioreactor (e.g., cells in the bioreactor). The process includes introducing an internal methyl transferase and internal methyl reductase into the bioreactor or a second bioreactor. The process includes obtaining an alkylated fatty acid having a methyl substituent located at an internal carbon atom of the fatty acid and a terminal methyl substituent or terminal ethyl substituent located at a carbon atom alpha to the terminal carbon atom of the fatty acid.

In at least one embodiment, a fatty acid ester has a methyl substituent. The fatty acid ester has (1) an ethyl substituent or (2) an additional methyl substituent, where the ethyl substituent or the additional methyl substituent is located at a carbon atom alpha to the terminal carbon atom of the fatty acid. The fatty acid ester optionally has an alcohol substituent.

In at least one embodiment, a lubricant includes a fatty acid ester.

Processes of the present disclosure can provide multiple-alkylated fatty acids and esters thereof. It has been discovered that methylation toward the middle of a fatty acid molecule (in addition to alkylation at a terminus of the fatty acid) is advantageous for cetane value and cold flow properties (likely because it is breaking up the waxy structure).

Furthermore, processes of the present disclosure can be beneficial because biological addition of methyl side chains eliminates the need to catalytically isomerize linear alkanes to obtain a branched structure, thus improving yield and removing the carbon-intensive and energy-intensive catalytic reforming process in the production of basestocks. The one or more methyl branches also provide useful physical properties to the alkanes.

Branched-chain fatty acids can have other varying properties when compared to straight-chain fatty acids of the same molecular weight (i.e., isomers), such as considerably lower melting points which can in turn provide lower pour points when made into industrial chemicals. These additional benefits allow the branched-chain fatty acids to confer substantially lower volatility and vapor pressure and improved stability against oxidation and rancidity. These properties make branched-chain fatty acids particularly suited as components for industrial lubricants.

Methylation at the fatty acid terminus alone does not significantly assist in breaking up the waxy properties promoted by the majority of the linear backbone of the fatty acid.

Fatty acid and ester products of the present disclosure can be formed at high yield, and conventional products are a mixture of methylated products.

The articles “a” and “an” are used herein to refer to one or to more than one (i.e., to at least one) of the grammatical object of the article. By way of example, “an element” means one element or more than one element.

The term “biologically-active portion” refers to an amino acid sequence that is less than a full-length amino acid sequence, but exhibits at least one activity of the full length sequence. For example, a biologically-active portion of a methyltransferase may refer to one or more domains of tmsB having biological activity for converting oleic acid (e.g., a phospholipid comprising an ester of oleate) and methionine (e.g., S-adenosyl methionine) into 10-methylenestearic acid (e.g., a phospholipid comprising an ester of 10-methylenestearate). A biologically-active portion of a reductase may refer to one or more domains of tmsA having biological activity for converting 10-methylenestearic acid (e.g., a phospholipid comprising an ester of 10-methylenestearate) and a reducing agent (e.g., NADH, NADPH, FAD, FADH₂, FMNH₂) into 10-methylstearic acid (e.g., a phospholipid comprising an ester of 10-methylstearate). Biologically-active portions of a protein include peptides or polypeptides comprising amino acid sequences sufficiently identical to or derived from the amino acid sequence of the protein which include fewer amino acids than the full length protein, and exhibit at least one activity of the protein, such as methyltransferase or reductase activity. A biologically-active portion of a protein may comprise, comprise at least, or comprise at most, for example, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more amino acids or any range derivable therein. Typically, biologically-active portions comprise a domain or motif having a catalytic activity. A biologically-active portion of a protein includes portions of the protein that have the same activity as the full-length peptide and every portion that has more activity than background. For example, a biologically-active portion of an enzyme may have, have at least, or have at most 0.1%, 0.5%, 1%, 2%, 3%, 4%, 5% 10%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%, 280%, 300%, 320%, 340%, 360%, 380%, 400% or higher activity relative to the full-length enzyme (or any range derivable therein). A biologically-active portion of a protein may include portions of a protein that lack a domain that targets the protein to a cellular compartment.

The terms “codon optimized” and “codon-optimized for the cell” refer to coding nucleotide sequences (e.g., genes) that have been altered to substitute at least one codon that is relatively rare in a desired host cell with a synonymous codon that is relatively prevalent in the host cell. Codon optimization thereby allows for better utilization of the tRNA of a host cell by matching the codons of a recombinant gene with the tRNA of the host cell. For example, the codon usage of the species of Actinobacteria (prokaryotes) varies from the codon usage of yeast (eukaryotes). The translation efficiency in a yeast host cell of an mRNA encoding an Actinobacteria protein may be increased by substituting the codons of the corresponding Actinobacteria gene with codons that are more prevalent in the particular species of yeast. A codon optimized gene thereby has a nucleotide sequence that varies from a naturally-occurring gene.

The term “constitutive promoter” refers to a promoter that mediates the transcription of an operably linked gene independent of a particular stimulus (e.g., independent of the presence of a reagent such as isopropyl β-D-1-thiogalactopyranoside).

The term “DGAT1” refers to a gene that encodes a type 1 diacylglycerol acyltransferase protein, such as a gene that encodes a yeast DGA2 protein.

The term “DGAT2” refers to a gene that encodes a type 2 diacylglycerol acyltransferase protein, such as a gene that encodes a yeast DGA1 protein.

“Diacylglyceride,” “diacylglycerol,” and “diglyceride” are esters comprised of glycerol and two fatty acids.

The terms “diacylglycerol acyltransferase” and “DGA” refer to any protein that catalyzes the formation of triacylglycerides from diacylglycerol. Diacylglycerol acyltransferases include type 1 diacylglycerol acyltransferases (DGA2), type diacylglycerol acyltransferases (DGA1), and type 3 diacylglycerol acyltransferases (DGA3) and all homologs that catalyze the above-mentioned reaction.

The terms “diacylglycerol acyltransferase, type 1” and “type 1 diacylglycerol acyltransferases” refer to DGA2 and DGA2 orthologs.

The terms “diacylglycerol acyltransferase, type 2” and “type 2 diacylglycerol acyltransferases” refer to DGA1 and DGA1 orthologs.

The term “domain” refers to a part of the amino acid sequence of a protein that is able to fold into a stable three-dimensional structure independent of the rest of the protein.

The term “drug” refers to any molecule that inhibits cell growth or proliferation, thereby providing a selective advantage to cells that contain a gene that confers resistance to the drug. Drugs include antibiotics, antimicrobials, toxins, and pesticides.

“Dry weight” and “dry cell weight” mean weight determined in the relative absence of water. For example, reference to oleaginous cells as comprising a specified percentage of a particular component by dry weight means that the percentage is calculated based on the weight of the cell after substantially all water has been removed. The term “% dry weight,” when referring to a specific fatty acid (e.g., oleic acid, lauroleic acid, or 10-methylstearic acid), includes fatty acids that are present as carboxylates, esters, thioesters, and amides. For example, a cell that comprises 10-methyl stearic acid as a percentage of total fatty acids by % dry cell weight includes 10-methyl stearic acid, 10-methylstearate, the 10-methylstearate portion of a diacylglycerol comprising a 10-methylstearate ester, the 10-methylstearate portion of a triacylglycerol comprising a 10-methylstearate ester, the 10-methylstearate portion of a phospholipid comprising a 10-methylstearate ester, and the 10-methylstearate portion of 10-methylstearate CoA. The term “% dry weight,” when referring to a specific type of fatty acid (e.g., C16 fatty acids, C18 fatty acids), includes fatty acids that are present as carboxylates, esters, thioesters, and amides as described above (e.g., for 10 methylstearic acid).

The term “encode” refers to nucleic acids that comprise a coding region, portion of a coding region, or compliments thereof. Both DNA and RNA may encode a gene. Both DNA and RNA may encode a protein.

The term “enzyme” as used herein refers to a protein that can catalyze a chemical reaction.

The term “expression” refers to the amount of a nucleic acid or amino acid sequence (e.g., peptide, polypeptide, or protein) in a cell. The increased expression of a gene refers to the increased transcription of that gene. The increased expression of an amino acid sequence, peptide, polypeptide, or protein refers to the increased translation of a nucleic acid encoding the amino acid sequence, peptide, polypeptide, or protein.

The term “gene,” as used herein, may encompass genomic sequences that contain exons, particularly polynucleotide sequences encoding polypeptide sequences involved in a specific activity. The term further encompasses synthetic nucleic acids that did not derive from genomic sequence. In certain embodiments, the genes lack introns, as they are synthesized based on the known DNA sequence of cDNA and protein sequence. In other embodiments, the genes are synthesized, non-native cDNA wherein the codons have been optimized for expression in Y. lipolytica or A. adeninivorans based on codon usage. The term can further include nucleic acid molecules comprising upstream, downstream, and/or intron nucleotide sequences.

The term “inducible promoter” refers to a promoter that mediates the transcription of an operably linked gene in response to a particular stimulus.

The term “integrated” refers to a nucleic acid that is maintained in a cell as an insertion into the cell's genome, such as insertion into a chromosome, including insertions into a plastid genome.

“In operable linkage” refers to a functional linkage between two nucleic acid. sequences, such a control sequence (typically a promoter) and the linked sequence (typically a sequence that encodes a protein, also called a coding sequence). A promoter is in operable linkage with a gene if it can mediate transcription of the gene.

The term “knockout mutation” or “knockout” refers to a genetic modification that prevents a native gene from being transcribed and translated into a functional protein.

The term “nucleic acid” refers to a polymeric form of nucleotides (also referred to as a “polynucleotide”) of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “phospholipid” refers to esters comprising glycerol, two fatty acids, and a phosphate. The phosphate may be covalently linked to carbon-3 of the glycerol and comprise no further substitution, e.g., the phospholipid may be a phosphatidic acid. The phosphate may be substituted with ethanol amine (e.g., phosphatidylethanolamine), choline (e.g., phosphatidylcholine), serine (e.g., phosphatidylserine), inositol (e.g., phosphatidylinositol), inositol phosphate (e.g., phosphatidylinositol-3-phosphate, phosphatidylinositol-4-phosphate, phosphatidylinositol-5-phosphate), inositol bisphosphate phosphatidylinositol-4,5-bisphosphate), or inositol triphosphate (e.g., phosphatidylinositol-3,4,5-bisphosphate).

As used herein, the term “plasmid” refers to a circular DNA molecule that is physically separate from an organism's genomic DNA. Plasmids may be linearized before being introduced into a host cell (referred to herein as a linearized plasmid). Linearized plasmids may not be self-replicating, but may integrate into and be replicated with the genomic DNA of an organism.

A “promoter” is a nucleic acid control sequence that directs the transcription of a nucleic acid. As used herein, a promoter includes the necessary nucleic acid sequences near the start site of transcription.

The term “protein” refers to molecules that comprise an amino acid sequence, wherein the amino acids are linked by peptide bonds.

“Transformation” refers to the transfer of a nucleic acid into a host organism or into the genome of a host organism, resulting in genetically stable inheritance. Host organisms containing the transformed nucleic acid are referred to as “recombinant,” “transgenic,” or “transformed” organisms. Thus, nucleic acids of the present disclosure can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. Typically, expression vectors include, for example, one or more cloned genes under the transcriptional control of 5′ and 3′ regulatory sequences and a selectable marker. Such vectors also can contain a promoter regulatory region (e.g., a regulatory region controlling inducible or constitutive, environmentally- or developmentally-regulated, or location-specific expression), a transcription initiation start site, a ribosome binding site, a transcription termination site, and/or a polyadenylation signal.

The term “transformed cell” refers to a cell that has undergone a transformation. Thus, a transformed cell comprises the parent's genome and an inheritable genetic modification.

The terms “triacylglyceride,” “triacylglycerol,” “triglyceride,” and “TAG” are esters comprised of glycerol and three fatty acids.

For the purposes of this present disclosure, and unless otherwise specified, all kinematic viscosity values in the present disclosure are as determined according to ASTM D445. Kinematic viscosity at 100° C. is reported herein as KV100, and kinematic viscosity at 40° C. is reported herein as KV40. Unit of all KV100 and KV40 values herein is cSt, unless otherwise specified.

For the purposes of this present disclosure, and unless otherwise specified, all viscosity index (VI) values in the present disclosure are as determined according to ASTM D2270.

For the purposes of this present disclosure, and unless otherwise specified, all Noack volatility (NV) values in the present disclosure are as determined according to ASTM D5800 and units of all NV values are wt %.

For the purposes of this present disclosure, and unless otherwise specified, rotating pressure vessel oxidation test (RPVOT) values in the present disclosure are determined according to ASTM D2272.

Microbe Engineering

A. Overview

Genes and gene products may be introduced into microbial host cells. Suitable host cells for expression of the genes and nucleic acid molecules are microbial hosts that can be found broadly within the fungal or bacterial families. Examples of suitable host strains include but are not limited to fungal or yeast species, such as Arxula, Aspergillus, Aurantiochytrium, Candida, Claviceps, Cryptococcus, Cunninghamella, Hansenula, Kluyveromyces, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Yarrowia, or bacterial species, such as members of proteobacteria and actinomycetes, as well as the genera Acinetobacter, Arthrobacter, Brevibacterium, Acidovorax, Bacillus, Clostridia, Streptomyces, Escherichia, Salmonella, Pseudomonas, and Cornyebacterium. Yarrowia lipolytica and Arxula adeninivorans are suited for use as a host microorganism because they can accumulate a large percentage of their weight as triacylglycerols.

Microbial expression systems and expression vectors containing regulatory sequences that direct high level expression of foreign proteins are known to those skilled in the art. Any of these could be used to construct chimeric genes to produce any one of the gene products of the instant sequences. These chimeric genes could then be introduced into appropriate microorganisms via transformation techniques to provide high-level expression of the enzymes.

For example, a gene encoding an enzyme can be cloned in a suitable plasmid, and an aforementioned starting parent strain as a host can be transformed with the resulting plasmid. This approach can increase the copy number of each of the genes encoding the enzymes and, as a result, the activities of the enzymes can be increased. The plasmid is not particularly limited so long as it renders a desired genetic modification inheritable to the microorganism's progeny.

Vectors or cassettes useful for the transformation of suitable host cells are well known. Typically the vector or cassette contains sequences that direct the transcription and translation of the relevant gene, a selectable marker, and sequences that allow autonomous replication or chromosomal integration. Suitable vectors comprise a region 5′ of the gene harboring transcriptional initiation controls and a region 3′ of the DNA fragment which controls transcriptional termination. In certain embodiments both control regions are derived from genes homologous to the transformed host cell, although it is to be understood that such control regions need not be derived from the genes native to the specific species chosen as a production host.

Promoters, cDNA, and 3′UTRs, as well as other elements of the vectors, can be generated through cloning techniques using fragments isolated from native sources (see, e.g., Green & Sambrook, Molecular Cloning: A Laboratory Manual, (4th ed., 2012); U.S. Pat. No. 4,683,202 (incorporated by reference)). Alternatively, elements can be generated synthetically using known methods (see, e.g., Gene 164:49-53 (1995)).

B. Homologous Recombination

Homologous recombination is the ability of complementary DNA sequences to align and exchange regions of homology. Transgenic DNA (“donor”) containing sequences homologous to the genomic sequences being targeted (“template”) is introduced into the organism and then undergoes recombination into the genome at the site of the corresponding homologous genomic sequences.

The ability to carry out homologous recombination in a host organism has many practical implications for what can be carried out at the molecular genetic level and is useful in the generation of a microbe that can produce a desired product. By its nature homologous recombination is a precise gene targeting event and, hence, most transgenic lines generated with the same targeting sequence will be essentially identical in terms of phenotype, necessitating the screening of far fewer transformation events. Homologous recombination also targets gene insertion events into the host chromosome, potentially resulting in excellent genetic stability, even in the absence of genetic selection. Because different chromosomal loci will likely impact gene expression, even from exogenous promoters/UTRs, homologous recombination can be a method of querying loci in an unfamiliar genome environment and to assess the impact of these environments on gene expression.

A particularly useful genetic engineering approach using homologous recombination is to co-opt specific host regulatory elements, such as promoters/UTRs, to drive heterologous gene expression in a highly specific fashion.

Because homologous recombination is a precise gene targeting event, it can be used to precisely modify any nucleotide(s) within a gene or region of interest, so long as sufficient flanking regions have been identified. Therefore, homologous recombination can be used as a means to modify regulatory sequences impacting gene expression of RNA and/or proteins. It can also be used to modify protein coding regions in an effort to modify enzyme activities such as substrate specificity, affinities and Km, thereby affecting a desired change in the metabolism of the host cell. Homologous recombination provides a powerful means to manipulate the host genome resulting in gene targeting, gene conversion, gene deletion, gene duplication, gene inversion, and exchanging gene expression regulatory elements such as promoters, enhancers and 3′UTRs.

Homologous recombination can be achieved by using targeting constructs containing pieces of endogenous sequences to “target” the gene or region of interest within the endogenous host cell genome. Such targeting sequences can either be located 5′ of the gene or region of interest, 3′ of the gene/region of interest or even flank the gene/region of interest. Such targeting constructs can be transformed into the host cell either as a supercoiled plasmid DNA with additional vector backbone, a PCR product with no vector backbone, or as a linearized molecule. In some cases, it may be advantageous to first expose the homologous sequences within the transgenic DNA (donor DNA) by cutting the transgenic DNA with a restriction enzyme. This step can increase the recombination efficiency and decrease the occurrence of undesired events. Other methods of increasing recombination efficiency include using PCR to generate transforming transgenic DNA containing linear ends homologous to the genomic sequences being targeted.

C. Vectors and Vector Components

Vectors for transforming microorganisms in accordance with the present disclosure can be prepared by known techniques familiar to those skilled in the art in view of the disclosure herein. A vector typically contains one or more genes, in which each gene codes for the expression of a desired product (the gene product) and is operably linked to one or more control sequences that regulate gene expression or target the gene product to a particular location in the recombinant cell.

1. Control Sequences

Control sequences are nucleic acids that regulate the expression of a coding sequence or direct a gene product to a particular location in or outside a cell. Control sequences that regulate expression include, for example, promoters that regulate transcription of a coding sequence and terminators that terminate transcription of a coding sequence. Another control sequence is a 3′ untranslated sequence located at the end of a coding sequence that encodes a polyadenylation signal. Control sequences that direct gene products to particular locations include those that encode signal peptides, which direct the protein to which they are attached to a particular location inside or outside the cell.

Thus, an exemplary vector design for expression of a gene in a microbe contains a coding sequence for a desired gene product (for example, a selectable marker, or an enzyme) in operable linkage with a promoter active in yeast. Alternatively, if the vector does not contain a promoter in operable linkage with the coding sequence of interest, the coding sequence can be transformed into the cells such that it becomes operably linked to an endogenous promoter at the point of vector integration.

The promoter used to express a gene can be the promoter naturally linked to that gene or a different promoter.

A promoter can generally be characterized as constitutive or inducible. Constitutive promoters are generally active or function to drive expression at all times (or at certain times in the cell life cycle) at the same level. Inducible promoters, conversely, are active (or rendered inactive) or are significantly up- or down-regulated only in response to a stimulus. Both types of promoters find application in the methods of the present disclosure. Inducible promoters useful in the present disclosure include those that mediate transcription of an operably linked gene in response to a stimulus, such as an exogenously provided small molecule, temperature (heat or cold), lack of nitrogen in culture media, etc. Suitable promoters can activate transcription of an essentially silent gene or upregulate, e.g., substantially, transcription of an operably linked gene that is transcribed at a low level.

Inclusion of termination region control sequence is optional, and if employed, then the choice is primarily one of convenience, as the termination region is relatively interchangeable. The termination region may be native to the transcriptional initiation region (the promoter), may be native to the DNA sequence of interest, or may be obtainable from another source (See, e.g., Chen & Orozco, Nucleic Acids Research 16:8411 (1988)).

2. Genes and Codon Optimization

Typically, a gene includes a promoter, a coding sequence, and termination control sequences. When assembled by recombinant DNA technology, a gene may be termed an expression cassette and may be flanked by restriction sites for convenient insertion into a vector that is used to introduce the recombinant gene into a host cell. The expression cassette can be flanked by DNA sequences from the genome or other nucleic acid target to facilitate stable integration of the expression cassette into the genome by homologous recombination. Alternatively, the vector and its expression cassette may remain unintegrated (e.g., an episome), in which case, the vector typically includes an origin of replication, which is capable of providing for replication of the vector DNA.

A common gene present on a vector is a gene that codes for a protein, the expression of which allows the recombinant cell containing the protein to be differentiated from cells that do not express the protein. Such a gene, and its corresponding gene product, is called a selectable marker or selection marker. Any of a wide variety of selectable markers can be employed in a transgene construct useful for transforming the organisms of the present disclosure.

For optimal expression of a recombinant protein, it is beneficial to employ coding sequences that produce mRNA with codons optimally used by the host cell to be transformed. Thus, proper expression of transgenes can require that the codon usage of the transgene matches the specific codon bias of the organism in which the transgene is being expressed. The precise mechanisms underlying this effect are many, but include the proper balancing of available aminoacylated tRNA pools with proteins being synthesized in the cell, coupled with more efficient translation of the transgenic messenger RNA (mRNA) when this need is met. When codon usage in the transgene is not optimized, available tRNA pools are not sufficient to allow for efficient translation of the transgenic mRNA resulting in ribosomal stalling and termination and possible instability of the transgenic mRNA. Resources for codon-optimization of gene sequences are described in Puigbo et al. (Nucleic Acids Research 35:W126-31 (2007)), and principles underlying codon optimization strategies are described in Angov (Biotechnology Journal 6:650-69 (2011)). Public databases providing statistics for codon usage by different organisms are available, including at www.kazusa.or.jp/codon/ and other publicly available databases and resources.

D. Transformation

Cells can be transformed by any suitable technique including, e.g., biolistics, electroporation, glass bead transformation, and silicon carbide whisker transformation. Any convenient technique for introducing a transgene into a microorganism can be employed in the present disclosure. Transformation can be achieved by, for example, the method of D. M. Morrison (Methods in Enzymology 68:326 (1979)), the method by increasing permeability of recipient cells for DNA with calcium chloride (Mandel & Higa, J. Molecular Biology, 53:159 (1970)), or the like.

Examples of expression of transgenes in oleaginous yeast (e.g., Yarrowia lipolytica) can be found in the literature (Bordes et al., J. Microbiological Methods, 70:493 (2007); Chen et al., Applied Microbiology & Biotechnology 48:232 (1997)). Examples of expression of exogenous genes in bacteria such as E. coli are well known (Green & Sambrook, Molecular Cloning: A Laboratory Manual, (4th ed., 2012)).

Vectors for transformation of microorganisms in accordance with the present disclosure can be prepared by known techniques familiar to those skilled in the art. In one embodiment, an exemplary vector design for expression of a gene in a microorganism contains a gene encoding an enzyme in operable linkage with a promoter active in the microorganism. Alternatively, if the vector does not contain a promoter in operable linkage with the gene of interest, the gene can be transformed into the cells such that it becomes operably linked to a native promoter at the point of vector integration. The vector can also contain a second gene that encodes a protein. Optionally, one or both gene(s) is/are followed by a 3′ untranslated sequence containing a polyadenylation signal. Expression cassettes encoding the two genes can be physically linked in the vector or on separate vectors. Co-transformation of microbes can also be used, in which distinct vector molecules are simultaneously used to transform cells (Protist 155:381-93 (2004)). The transformed cells can be optionally selected based upon the ability to grow in the presence of the antibiotic or other selectable marker under conditions in which cells lacking the resistance cassette would not grow.

Nucleic Acids

Various aspects of the disclosure relate to a nucleic acid comprising an unmodified methyltransferase gene or a recombinant methyltransferase gene, an unmodified reductase gene or a recombinant reductase gene, an unmodified terminal alkyl transferase gene or a recombinant terminal alkyl transferase gene, or combination(s) thereof. The nucleic acid may be, for example, a plasmid. In some embodiments, a methyltransferase gene, a reductase gene, and/or terminal alkyl transferase gene is integrated into the genome of a cell, and thus, the nucleic acid may be a chromosome. In some embodiments, the disclosure relates to a cell comprising a methyltransferase gene, a reductase gene, and/or terminal alkyl transferase gene, e.g., wherein the methyltransferase gene, reductase gene, and/or terminal alkyl transferase gene is present in a plasmid or chromosome. A methyltransferase gene, reductase gene, and/or terminal alkyl transferase gene may be present in a cell in the same nucleic acid (e.g., same plasmid or chromosome) or in different nucleic acids (e.g., different plasmids or chromosomes).

A nucleic acid may be inheritable to the progeny of a transformed cell. A gene such as a methyltransferase gene, reductase gene, and/or terminal alkyl transferase gene may be inheritable because it resides on a plasmid or chromosome. In certain embodiments, a gene may be inheritable because it is integrated into the genome of the transformed cell.

A gene may comprise conservative substitutions, deletions, and/or insertions while still encoding a protein that has activity. For example, codons may be optimized for a particular host cell, different codons may be substituted for convenience, such as to introduce a restriction site or to create optimal PCR primers, or codons may be substituted for another purpose. Similarly, the nucleotide sequence may be altered to create conservative amino acid substitutions, deletions, and/or insertions.

Proteins may comprise conservative substitutions, deletions, and/or insertions while still maintaining activity. Conservative substitution tables are well known in the art (Creighton, Proteins (2d. ed., 1992)).

Amino acid substitutions, deletions and/or insertions may readily be made using recombinant DNA manipulation techniques. Methods for the manipulation of DNA sequences to produce substitution, insertion or deletion variants of a protein are well known in the art. These methods include M13 mutagenesis, T7-Gen in vitro mutagenesis (USB, Cleveland, Ohio), uick Change Site Directed mutagenesis (Stratagene, San Diego, Calif.), PCR-mediated site-directed mutagenesis, and other site-directed mutagenesis protocols.

To determine the percent identity of two amino acid sequences or two nucleic acid sequences, the sequences can be aligned for optimal comparison purposes (e.g., gaps can be introduced in one or both of a first and a second amino acid or nucleic acid sequence for optimal alignment and non-identical sequences can be disregarded for comparison purposes). The length of a reference sequence aligned for comparison purposes can be at least 95% of the length of the reference sequence. The amino acid residues or nucleotides at corresponding amino acid positions or nucleotide positions can then be compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. Unless otherwise specified, when percent identity between two amino acid sequences is referred to herein, it refers to the percent identity as determined using the Needleman and Wunsch (J. Molecular Biology 48:444-453 (1970)) algorithm which has been incorporated into the GAP program in the GCG software package (available at http://www.gcg.com), using a Blosum 62 matrix, a gap weight of 10, and a length weight of 4. In some embodiments, the percent identity between two amino acid sequences is determined the Needleman and Wunsch algorithm using a Blosum 62 matrix or a PAM250 matrix, and a gap weight of 16, 14, 12, 10, 8, 6, or 4 and a length weight of 1, 2, 3, 4, 5, or 6. Unless otherwise specified, when percent identity between two nucleotide sequences is referred to herein, it refers to percent identity as determined using the GAP program in the GCG software package (available at http://www.gcg.com), using a NWSgaptina.CMP matrix and a gap weight of 60 and a length weight of 4. In yet another embodiment, the percent identity between two nucleotide sequences can be determined using a gap weight of 40, 50, 60, 70, or 80 and a length weight of 1, 2, 3, 4, 5, or 6. In another embodiment, the percent identity between two amino acid or nucleotide sequences can be determined using the algorithm of E. Meyers and W. Miller (Computer Applications in the Biosciences 4:11-17 (1988)) which has been incorporated into the ALIGN program (version 2.0 or 2.0U), using a PAM120 weight residue table, a gap length penalty of 12 and a gapp enalty of 4.

Exemplary computer programs which can be used to determine identity between two sequences include, but are not limited to, the suite of BLAST programs, e.g., BLASTN, MEGABLAST, BLASTX, TBLASTN, TBLASTX, and BLASTP, and Clustal programs, ClustalW, ClustalX, and Clustal Omega.

Sequence searches are typically carried out using the BLASTN program, when evaluating a given nucleic acid sequence relative to nucleic acid sequences in the GenBank DNA Sequences and other public databases. The BLASTX program is effective for searching nucleic acid sequences that have been translated in all reading frames against amino acid sequences in the GenBank Protein Sequences and other public databases.

An alignment of selected sequences in order to determine “% identity” between two or more sequences is performed using for example, the CLUSTAL-W program.

A “coding sequence” or “coding region” refers to a nucleic acid molecule having sequence information necessary to produce a protein product, such as an amino acid or polypeptide, when the sequence is expressed. The coding sequence may comprise and/or consist of untranslated sequences (including introns or 5′ or 3′ untranslated regions) within translated regions, or may lack such intervening untranslated sequences (e.g., as in cDNA).

The abbreviation used throughout the specification to refer to nucleic acids comprising and/or consisting of nucleotide sequences are the conventional one-letter abbreviations. Thus when included in a nucleic acid, the naturally occurring encoding nucleotides are abbreviated as follows: adenine (A), guanine (G), cytosine (C), thymine (T) and uracil (U). Also, unless otherwise specified, the nucleic acid sequences presented herein is the 5′→3′ direction.

As used herein, the term “complementary” and derivatives thereof are used in reference to pairing of nucleic acids by the well-known rules that A pairs with T or U and C pairs with G. Complement can be “partial” or “complete”. In partial complement, only some of the nucleic acid bases are matched according to the base pairing rules; while in complete or total complement, all the bases are matched according to the pairing rule. The degree of complement between the nucleic acid strands may have significant effects on the efficiency and strength of hybridization between nucleic acid strands as well known in the art. The efficiency and strength of said hybridization depends upon the detection method.

Any nucleic acid that is referred to herein as having a certain percent sequence identity to a sequence set forth in a SEQ ID NO, includes nucleic acids that have the certain percent sequence identity to the complement of the sequence set forth in the SEQ ID NO.

Exemplary Cells, Nucleic Acids, Compositions, and Methods for Internal Fatty Acid Methylation

A. Cell

In some embodiments, the cell (e.g., transformed cell or unmodified cell) is a prokaryotic cell, such as a bacterial cell. In some embodiments, the cell is a eukaryotic cell, such as a mammalian cell, a yeast cell, a filamentous fungi cell, a protist cell, an algae cell, an avian cell, a plant cell, or an insect cell. In some embodiments, the cell is a yeast. Those with skill in the art will recognize that many forms of filamentous fungi produce yeast-like growth, and the definition of yeast herein encompasses such cells. The cell may be selected from the group consisting of algae, bacteria, molds, fungi, plants, and yeasts. The cell may be a yeast, fungus, or yeast-like algae. The cell may be selected from thraustochytrids (Aurantiochytrium) and achlorophylic unicellular algae (Prototheca).

The cell may be selected from the group consisting of Arxula, Aspergillus, Aurantiochytrium, Candida, Claviceps, Cryptococcus, Cunninghamella, Escherichia, Geotrichum, Hansenuta, Kluyveromyces, Kodamaea, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Wickerhamomyces, and Yarrowia.

The cell may be selected from the group of consisting of Arxula adeninivorans, Aspergillus niger, Aspergillus orzyae, Aspergillus terreus, Aurantiochytrium limacinum, Candida utilis, Claviceps purpurea, Cryptococcus albidus, Cryptococcus curvatus, Cryptococcus ramirezgomezianus, Cryptococcus terretts, Cryptococcus wieringae, Cunninghamella echinulata, Cunninghamella japonica Geotrichum fermentans, Hansenula polymorpha, Kluyverontyces lactis, Kluyveromyces marxianus, Kodamaea ohtneri, Leucosporidiella creatinivora, Lipomyces lipofer, Lipomyces starkeyi, Lipomyces tetrasporus, Mortierella isabellina, Mortierella alpina, Ogataea polymorpha, Pichia ciferrii, Pichia guilliermondii, Pichia pastoris, Pichia stipites, Prototheca zopfii, Rhizopus arrhizus, Rhodosporidium babjevae, Rhodosporidium toruloides, Rhodosporidium paludigenum, Rhodotorula glutinis, Rhodotorula mucilaginosa, Saccharomyces cerevisiae, Schizosaccharomyces pombe, Tremella enchepala, Trichosporon cutaneum, Trichosporon fermentans, Wickerhamomyces ciferrii, and Yarrowia lipolytica.

In at least one embodiment, the cell may be Saccharomyces cerevisiae, Yarrowia lipolytica, or Arxula adeninivorans.

In certain embodiments, the cell comprises at least 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, or more lipid as measured by % dry cell weight, or any range derivable therein. In some embodiments, the cell comprises C18 fatty acids at a concentration of at least 5%, 10%, 15% 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 7⁶%, 77%, 78%, 79%, 80%, 81%, 82%, 83%, 84%, 85%, 86%, 87%, 88%, 89%, 90%, 91%, 92%, 93%, 94%, 95%, or higher as a percentage of total C16 and C18 fatty acids in the cell, or any range derivable therein.

In some embodiments, the cell comprises oleic acid at a concentration of at least 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% 75% 76%, 77% 78%, 79%, 80%, 81%, 82%, 83%, 84%, 85%, 86%, 87%, 88%, 89%, 90%, or higher as a percentage of total C16 and C18 fatty acids in the cell, or any range derivable therein. In some embodiments, the cell comprises a linear fatty acid with a chain length of 12-20 carbons with a methyl branch at the 8, 9, 10, 11, or 12 position at a concentration of at least 1%, 2%, 3%, 4%, 5%, 6%, 7% 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45% 46%, 47% 48%, 49% 50%, 51%, 52%, 53%, 54% 55%, 56%, 57%, 5⁸%, 59% 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, or 99% by weight or higher as a percentage of total fatty acids in the cell, or any range derivable therein. In some embodiments, the fatty acid has a chain length of 12, 13, 14, 15, 16, 17, 18, 19, or 20 carbons, or any range derivable therein.

A cell may be modified to increase its oleate content, which serves as a substrate for 10-methylstearate synthesis. Genetic modifications that increase oleate content are known (see, e.g., PCT Patent Application Publication No. WO16/094520, published Jun. 16, 2016, hereby incorporated by reference). For example, a cell may comprise a Δ12 desaturase knockdown or knockout, which favors the accumulation of oleate and disfavors the production of linoleate. A cell may comprise a recombinant Δ9 desaturase gene, which favors the production of oleate and disfavors the accumulation of stearate. The recombinant Δ9 desaturase gene may be, for example, the Δ9 desaturase gene from Y. lipolytica, Arxula adeninivorans, or Puccinia graminis. A cell may comprise a recombinant elongase 1 gene, which favors the production of oleate and disfavors the accumulation of palmitate and palmitoleate. The recombinant elongase 1 gene may be the elongase 1 gene from Y. lipolytica. A cell may comprise a recombinant elongase 2 gene, which favors the production of oleate and disfavors the accumulation of palmitate and palmitoleate. The recombinant elongase 2 gene may be the elongase 2 gene from R. norvegicus.

A cell may be modified to increase its triacylglycerol content, thereby increasing its 10-methylstearate content. Genetic modifications that increase triacylglycerol content are known (see, e.g., PCT Patent Application Publication No. WO16/094520, published Jun. 16, 2016, hereby incorporated by reference). A cell may comprise a recombinant diacylglycerol acyltransferase gene (e.g., DGAT1, DGAT2, or DGAT3), which favors the production of triacylglycerols and disfavors the accumulation of diacylglycerols. The recombinant di acylglycerol acyltransferase gene may be, for example, DGAT2 (encoding protein DGA1) from Y. lipolytica, DGAT (encoding protein DGA2) from C. purpurea, or DGAT2 (encoding protein DGA1) from R. toruloides. The cell may comprise a glycerol-3-phosphate acyltransferase gene (sct1) knockdown or knockout, which may favor the accumulation of triacylglycerols, depending on the cell type. The cell may comprise a recombinant glycerol-3-phosphate acyltransferase gene (sct1) such as the sct1 gene from A. adeninivorans, which may favor the accumulation of triacylglycerols. The cell may comprise a triacylglycerol lipase gene (tgl) knockdown or knockout, which may favor the accumulation of triacylglycerols in the cell.

Various aspects of the present disclosure relate to a transformed cell. The transformed cell may comprise a recombinant methyltransferase gene (e.g., a tmsB gene), a recombinant reductase gene (e.g., a tmsA gene), an exomethylene-substituted lipid, and/or a branched (methyl)lipid. A transformed cell may comprise a tmsC gene. A branched (methyl)lipid may be a carboxylic acid (e.g., 8-methyl-lauroleic acid, 10-methylstearic acid, 10-methylpalmitic acid, 12-methyloleic acid, 13-methyloleic acid, 10-methyl-octadec-12-enoic acid), carboxylate (e.g., 10-methylstearate, 10-methylpalmitate, 12-methyloleate, 13-methyloleate, 10-methyl-octadec-12-enoate), ester (e.g., diacylglycerol, triacylglycerol, phospholipid), thioester (e.g., 10-methylstearyl CoA, 10-methylpalmityl CoA, 12-methyloleoyl CoA, 13-methyloleoyl CoA, 10-methyl-octadec-12-enoyl CoA), or amide. An exomethylene-substituted lipid may be a carboxylic acid (e.g., 8-methylene-lauroleic acid, 10-methylenestearic acid, 10-methylenepalmitic acid, 12-methyleneoleic acid, 13-methyleneoleic acid, 10-methylene-octadec-12-enoic acid), carboxylate (e.g., 10-methylenestearate, 10-methylenepalmitate, 12-methyleneoleate, 13-methyleneoleate, 10-methylene-octadec-12-enoate), ester (e.g., diacylglycerol, triacylglycerol, phospholipid), thioester (e.g., 10-methylenestearyl CoA, 10-methylenepalmityl CoA, 12-methyleneoleoyl CoA, 13-methyleneoleoyl CoA, 10-methylene-octadec-12-enoyl CoA), or amide.

“Fatty acids” generally exist in a cell as a phospholipid or triacylglycerol, although they may also exist as a monoacylglycerol or diacylglycerol, for example, as a metabolic intermediate. Free fatty acids also exist in the cell in equilibrium between a relatively abundant carboxylate anion and a relatively scarce, neutrally-charged acid. A fatty acid may exist in a cell as a thioester, especially as a thioester with coenzyme A (CoA), during biosynthesis or oxidation. A fatty acid may exist in a cell as an amide, for example, when covalently bound to a protein to anchor the protein to a membrane.

A cell may comprise nucleic acids.

A branched (methyl)lipid may comprise a saturated branched aliphatic chain (e.g., 8-methyl-lauroleic acid, 10-methylstearic acid, 10-methylpalmitic acid) or an unsaturated branched aliphatic chain 12-methyloleic acid, 13-methyloleic acid, 10-methyl-octadec-12-enoic acid). The branched (methyl)lipid may comprise a saturated or unsaturated branched aliphatic chain comprising a branching methyl group.

An exomethylene-substituted lipid may comprise a branched aliphatic chain (e.g., 8-methylene-lauroleic acid, 10-methylenestearic acid, 10-methylenepalmitic acid, 12-methyleneoleic acid, 13-methyleneoleic acid, 10-methylene-octadec-12-enoic acid). The aliphatic chain may be branched because the aliphatic chain is substituted with an exomethylene group.

A branched (methyl)lipid may be 8-methyl-laurolate, 10-methylstearate, or an acid (8-methyl-lauroleic acid or 10-methyl stearic acid), ester (e.g., diacylglycerol, triacylglycerol, phospholipid), thioester (e.g., 10-methylstearyl CoA), or amide (e.g., 10-methylstearyl amide) thereof. For example, the branched (methyl)lipid may be a diacylglycerol, triacylglycerol, or phospholipid, and the diacylglycerol, triacylglycerol, or phospholipid may comprise an ester of 10-methyl stearate.

An exomethylene-substituted lipid may be 8-methylene-laurolate or 10-methylenestearate, or an acid (8-methylene-lauroleic acid or 10-methylenestearic acid), ester (e.g., diacylglycerol, triacylglycerol, phospholipid), thioester (e.g., 10-methylenestearyl CoA), or amide (e.g., 10-methylenestearyl amide) thereof. For example, the exomethylene-substituted lipid may be a diacylglycerol, triacylglycerol, or phospholipid, and the diacylglycerol, triacylglycerol, or phospholipid may comprise an ester of 10-methylenestearate.

In some embodiments, about, at most about, or at least about 1% of the fatty acids of the cell may be 10-methylstearic acid as measured by % dry cell weight. About, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the fatty acids of the cell may be 10-methylstearic acid as measured by % dry cell weight, or any range derivable therein.

In some embodiments, about, at least about, or at most about 1% of the fatty acids of the cell may be 10-methylenestearic acid as measured by % dry cell weight. About, at least about, or at most about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 67%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the fatty acids of the cell may be 10-methylenestearic acid as measured by % dry cell weight, or any range derivable therein.

In some embodiments, about, at least about, or at most about 1% by weight of the fatty acids of the cell may be one or more of the branched (methyl)lipids described herein. About, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 75%, 6%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57% 58%, 59% 60%, 61%, 62%, 63%, 64%, 65%, 66%, 67%, 68%, 69%, 70%, 71%, 72%, 73%, 74%, 75%, 76%, 77%, 78%, 79%, 80%, 81%, 87%, 83%, 84%, 85%, 86%, 87%, 88%, 89%, 90%, 91%, 92%, 93%, 94%, 95%, 96%, 97%, 98%, or 99% by weight of the fatty acids of the cell may be one or more of the branched (methyl)lipids described herein, or any range derivable therein.

In some embodiments, about, at least about, or at most about 1% by weight of the fatty acids of the cell may be one or more of the branched (methyl)lipids described herein (e.g., a linear fatty acid with a chain length of 12-20 carbons with a methyl branch at the 8, 9, 10, 11, or 12 position). About, at least about, or at most about 2%, 3%, 4%, 5%, 6%, 7%, 8%, 9%, 10%, 11%, 12%, 13%, 14%, 15%, 16%, 17%, 18%, 19%, 20%, 71%, 22%, 23%, 24%, 25%, 26%, 27%, 28%, 29%, 30%, 31%, 32%, 33% 34%, 35% 36%, 37%, 38%, 39%, 40%, 41%, 42%, 43%, 44%, 45%, 46%, 47%, 48%, 49%, 50%, 51%, 52%, 53% 54% 55%, 56%, 57% 58%, 59% 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% 94%, 95% 96%, 97%, 98%, or 99% of the fatty acids of the cell may be one or more of the branched (methyl)lipids described herein (e.g., a linear fatty acid with a chain length of 12-20 carbons with a methyl branch at the 8, 9, 10, or 11, or 12 position), or any range derivable therein.

In some embodiments, the cell may comprise about, at least about, or at most about 1% 10-methylstearic acid as measured by % dry cell weight. The cell may comprise about, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% 10-methyl stearic acid as measured by % dry cell weight, or any range derivable therein.

In some embodiments, the cell may comprise about, at least about, or at most about 1% 10-methylenestearic acid as measured by % dry cell weight. The cell may comprise about, at least about, or at most about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% 10-methylenestearic acid as measured by % dry cell weight, or any range derivable therein.

An unmodified cell of the same type (e.g., species) as a cell of the present disclosure may comprise 8-methyl-laurolate or 10-methylstearate, or an acid (8-methyl-lauroleic acid or 10-methylstearic acid), ester (e.g., diacylglycerol, triacylglycerol, phospholipid), thioester (e.g., 10-methylstearyl CoA), or amide (e.g., 10-methylstearyl amide) thereof (e.g., wherein the unmodified cell does not comprise a recombinant methyltransferase gene or a recombinant reductase gene). An unmodified cell of the same type (e.g., species) as a transformed cell may comprise 8-methylene-laurolate or 10-methylenestearate, or an acid (8-methylene-lauroleic acid or 10-methylenestearic acid), ester (e.g., diacylglycerol, triacylglycerol, phospholipid), thioester (e.g., 10-methylenestearyl CoA), or amide (e.g., 10-methylenestearyl amide) thereof (e.g., wherein the unmodified cell does not comprise a recombinant methyltransferase gene or a recombinant reductase gene). In some embodiments, an unmodified cell of the same species as the cell comprises a branched (methyl)lipid and/or an exomethylene-substituted lipid. In some embodiments, an unmodified cell of the same species as the cell comprises one or more of the branched (methyl)lipids or exomethylene-substituted lipids described herein.

A cell may constitutively express the protein encoded by a methyltransferase gene. A cell may constitutively express the protein encoded by a reductase gene. A cell may constitutively express the protein encoded by a tmsC gene. A cell may constitutively express a methyltransferase protein. A cell may constitutively express a reductase protein. A cell may constitutively express a TmsC protein.

i. Nucleic Acids Comprising a Methyltransferase Gene

A methyltransferase gene (e.g., an unmodified or recombinant methyltransferase gene) encodes a methyltransferase protein, which is an enzyme capable of transferring a carbon atom and one or more protons bound thereto from a substrate such as S-adenosyl methionine to a fatty acid such as oleic acid (e.g., wherein the fatty acid is present as a free fatty acid, carboxylate, phospholipid, diacylglycerol, or triacylglycerol). A methyltransferase gene (e.g., an unmodified or recombinant methyltransferase gene) may comprise any one of the nucleotide sequences set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124. A methyltransferase gene (e.g., an unmodified or recombinant methyltransferase gene) may be a 10-methylstearic B gene (tmsB) as described herein, or a biologically-active portion thereof (i.e., wherein the biologically-active portion thereof comprises methyltransferase activity).

A methyltransferase gene (e.g., an unmodified or recombinant methyltransferase gene) may be derived from a gram-positive species of Actinobacteria, such as Mycobacteria, Corynebacteria, Nocardia, Streptomyces, or Rhodococcus. A methyltransferase gene may be selected from the group consisting of Mycobacterium smegmatis gene tmsB, Agromyces subbeticus gene tmsB, Amycolicicoccus subflavus tmsB, Corynebacterium glutamicum gene tmsB, Corynebacterium glyciniphilium gene tmsB, Knoella aerolata gene tmsB, Mycobacterium austroafricanum gene tmsB, Mycobacterium gilvum gene tmsB, Mycobacterium indicus pranii gene tmsB, Mycobacterium phlei gene tmsB, Mycobacterium tuberculosis gene tmsB, Mycobacterium vanbaalenii gene tmsB, Rhodococcus opacus gene tmsB, Streptomyces regnsis gene tmsB, Thermobifida fusca gene tmsB, and Thermomonospora curvata gene tmsB.

A recombinant methyltransferase gene may be recombinant because it is operably-linked to a promoter other than the naturally-occurring promoter of the methyltransferase gene. Such genes may be useful to drive transcription in a particular species of cell. A recombinant methyltransferase gene may be recombinant because it contains one or more nucleotide substitutions relative to a naturally-occurring methyltransferase gene. Such genes may be useful to increase the translation efficiency of the methyltransferase gene's mRNA transcript in a particular species of cell.

A nucleic acid may comprise a recombinant methyltransferase gene and a promoter, wherein the recombinant methyltransferase gene and promoter are operably-linked. The methyltransferase gene and promoter may be derived from different species. For example, the methyltransferase gene may encode the methyltransferase protein of a gram-positive species of Actinobacteria, and the methyltransferase gene may be operably-linked to a promoter that can drive transcription in another phylum of bacteria (e.g., a Proteobacterium, such as E. coli) or a eukaryote an algae cell, yeast cell, or plant cell). The promoter may be a eukaryotic promoter. A cell may comprise the nucleic acid, and the promoter may be capable of driving transcription in the cell. A cell may comprise a methyltransferase gene, and the methyltransferase gene may be operably-linked to a promoter capable of driving transcription of the methyltransferase gene in the cell. The cell may be a species of yeast, and the promoter may be ayeast promoter. The cell may be a species of bacteria, and the promoter may be a bacterial promoter (e.g., wherein the bacterial promoter is not a promoter from Actinobacteria). The cell may be a species of algae, and the promoter may be an algae promoter. The cell may be a species of plant, and the promoter may be a plant promoter.

A methyltransferase gene may be operably-linked to a promoter that cannot drive transcription in the cell from which the methyltransferase gene originated. For example, the promoter may not be capable of binding an RNA polymerase of the cell from which a methyltransferase gene originated. In some embodiments, the promoter cannot bind a prokaryotic RNA polymerase and/or initiate transcription mediated by a prokaryotic RNA polymerase. In some embodiments, a methyltransferase gene is operably-linked to a promoter that cannot drive transcription in the cell from which the protein encoded by the gene originated. For example, the promoter may not be capable of binding an RNA polymerase of a cell that naturally expresses the methyltransferase enzyme encoded by a methyltransferase gene.

A promoter may be an inducible promoter or a constitutive promoter. A promoter may be any one of the promoters described in PCT Patent Application Publication No. WO 2016/014900, published Jan. 28, 2016 (hereby incorporated by reference in its entirety). WO 2016/014900 describes various promoters derived from yeast species Yarrowia lipolytica and Arxula adeninivorans, which may be particularly useful as promoters for driving the transcription of a gene in a yeast cell. A promoter may be a promoter from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pᵢ cotransporter; Pyruvate decarboxylase; Phytase; or Alpha-amylase, e.g., wherein the gene is a yeast gene, such as a gene from Yarrowia lipolytica or Arxula adeninivorans.

A methyltransferase gene may comprise a nucleotide sequence with at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124. A methyltransferase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity (or any range derivable therein) with 150, 200, 250, 300, 350, 400, 450, 500, 550, 600, 650, 700, 750, 800, 850, 900, 950, 1000, 1050, 1100, 1150, 1200, 1250, or 1300 contiguous base pairs (or any range derivable therein) starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124. A methyltransferase gene may or may not have 100% sequence identity with any one of the nucleotide sequences set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124. A methyltransferase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124. A methyltransferase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124, and the methyltransferase gene may encode a methyltransferase protein with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, 99%, or 100% sequence identity with the amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125. For example, even though SEQ ID NO:2 and SEQ ID NO:4 do not have 100% sequence identity, the two nucleotide sequences may encode the same amino acid sequence.

A recombinant methyltransferase gene may vary from a naturally-occurring methyltransferase gene because the recombinant methyltransferase gene may be codon-optimized for expression in a eukaryotic cell, such as a plant cell, algae cell, or yeast cell. A cell may comprise a recombinant methyltransferase gene, wherein the recombinant methyltransferase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant methyltransferase gene may vary from a naturally-occurring methyltransferase gene or may be unchanged from a naturally-occurring methyltransferase gene. For example, a recombinant methyltransferase gene may comprise a nucleotide sequence with at least about 65% sequence identity with the naturally-occurring nucleotide sequence set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124 (e.g., at least 70%, 75%, 80%, 85%, 90%, 95%, 96%, 97%, 98%, or 99% sequence identity), and at least 5 codons of the nucleotide sequence of the recombinant methyltransferase gene may vary from the naturally-occurring nucleotide sequence (e.g., at least 10, 15, 20, 25, 30, 35, 40, 45, 50, 60, 70, 80, 90, or 100 codons (or any range derivable therein)).

A methyltransferase gene encodes a methyltransferase protein. A methyltransferase protein may be a protein expressed by a gram-positive species of Actinobacteria, such as Bacillus, Haemophilus, Vibrio harvevi, Rhodobacter, Escherichia, Staphylococci, Streptomycete, or Corynebacteria. A recombinant methyltransferase gene may encode a naturally-occurring methyltransferase protein even if the recombinant methyltransferase gene is not a naturally-occurring methyltransferase gene. For example, a recombinant methyltransferase gene may vary from a naturally-occurring methyltransferase gene because the recombinant methyltransferase gene is codon-optimized for expression in a specific cell. The codon-optimized, recombinant methyltransferase gene and the naturally-occurring methyltransferase gene may nevertheless encode the same naturally-occurring methyltransferase protein.

A recombinant methyltransferase gene may encode a methyltransferase protein selected from Mycobacterium smegmatis enzyme TmsB, Agromyces subbeticus enzyme TmsB, Amycolicoccus subflavus enzyme TmsB, Corynebacterium glutamicum enzyme TmsB, Corynebacterium glyciniphilium enzyme TmsB, Knoella aerolata enzyme TmsB, Mycobacterium austroafricanum enzyme TmsB, Mycobacterium gilvum enzyme TmsB, Mycobacterium indicus pranii enzyme TmsB, Mycobacterium phlei enzyme TmsB, Mycobacterium tuberculosis enzyme TmsB, Mycobacterium vanbaalenii enzyme TmsB, Rhodococcus opacus enzyme TmsB, Streptomyces regnsis enzyme TmsB, Thermobifida fusca enzyme TmsB, and Thermomonospora curvata enzyme TmsB. A methyltransferase gene may encode a methyltransferase protein, and the methyltransferase protein may be substantially identical to any one of the foregoing enzymes, but a recombinant methyltransferase gene may vary from the naturally-occurring gene that encodes the enzyme. The recombinant methyltransferase gene may vary from the naturally-occurring gene because the recombinant methyltransferase gene may be codon-optimized for expression in a specific phylum, class, order, family, genus, species, or strain of cell.

The sequences of naturally-occurring methyltransferase proteins are set forth in SEQ ID NO:1, SEQ ID No:3, SEQ ID No:5, SEQ ID No:7, SEQ ID No:9, SEQ ID No:11, SEQ ID No:13, SEQ ID No:15, SEQ ID No:17, SEQ ID No:19, SEQ ID No:21, SEQ ID No:23, SEQ ID No:25, SEQ ID No:27, SEQ ID No:29, SEQ ID No:31, SEQ ID No:116, or SEQ ID No:124. A recombinant methyltransferase gene may or may not encode a protein comprising 100% sequence identity with the amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125. For example, a recombinant methyltransferase gene may encode a protein having 100% sequence identity with a biologically-active portion of an amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125.

A recombinant methyltransferase gene may encode a methyltransferase protein having, having at least, or having at most 65%, 66%, 67%, 68%, 69%, 70%, 71%, 72%, 73%, 74%, 75%, 76%, 77% 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity (or any range derivable therein) with the amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125, or a biologically-active portion thereof. A recombinant methyltransferase gene may encode a methyltransferase protein having at least about 0.1%, 0.5%, 1%, 2%, 3%, 4%, 5% 10%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95% 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%, 280%, 300%, 320%, 340%, 360%, 380%, or 400% methyltransferase activity (or any range derivable therein) relative to a protein comprising the amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125. A recombinant methyltransferase gene may encode a protein having at least 70%, 75%, 80%, 85%, 90%, 91%, 92%, 93%, 94%, 95%, 96%, 97%, 98%, 99%, 99.9%, or 100% sequence identity with 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous amino acids starting at amino acid position 1, 2, 3, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 of SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID NO:125.

Substrates for the methyltransferase protein may include any fatty acid from 12 to 20 carbons long with an unsaturated double bond in the Δ7, Δ8, Δ9, Δ10, or Δ11 position. The methyltransferase protein may be capable of catalyzing the formation of a methylene substitution at the 8, 9, 10, 11, or 12 position of such a substrate.

In some embodiments, the recombinant methyltransferase gene encodes a methyltransferase protein that includes an S-adenosylmethionine-dependent methyltransferase domain. In some embodiments, the S-adenosylmethionine-dependent methyltransferase domain has, has at least, or has at most 70%, 75%, 80%, 85%, 90%, 91%, 92%, 93%, 94%, 95%, 96%, 97%, 98%, 99%, 99.9%, or 100% sequence identity to amino acids 192-291 of T. curvata TmsB (SEQ ID NO:32) or to a corresponding portion of TmsB from Mycobacterium smegmatis, Mycobacterium vanbaaleni, Amycolicicoccus subflavus, Corynebacterium glyciniphilium, Corynebacterium glutamicum, Rhodococcus opacus, Agromyces subbeticus, Knoellia aerolata, Mycobacterium gilvum, Mycobacterium sp. Indicus, or Thermobifida fusca.

In some embodiments, the recombinant methyltransferase gene encodes a methyltransferase protein that has specific amino acids unchanged from the amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125. The unchanged amino acids can include 1, 3, 4, 5, 6, 7, 8, 9, 10, 11, 12, 13, 14, 15, 16, 17, 18, 19, 20, 21, 22, 23, 24, 25, 26, 27, 28, or 29 amino acids selected from D23, G24, A59, H128, F147, Y148, L180, L193, M203, G236, A241, R313, R318, E320, L359, L400, V196, G197, 0198, G199, W200, G201, G202, T219, L220, Q246, D247, Y248, and D262 of T. curvata TmsB (SEQ ID NO:32) or corresponding amino acids in TmsB from Mycobacterium smegmatis, Mycobacterium vanbaaleni, Amycolicicoccus subflavus, Corynebacterium glyciniphilium, Corynebacterium glutamicum, Rhodococcus opacus, Agromyces subbeticus, Knoellia aerolata, Mycobacterium gilvum, Mycobacterium sp. Indicus, or Thermobifida fusca.

ii. Nucleic Acids Comprising a Reductase Gene

A reductase gene (e.g., an unmodified reductase gene or recombinant reductase gene) encodes a reductase protein, which is an enzyme capable of reducing, often in an NADPH-dependent manner, a double bond of a fatty acid (e.g., wherein the fatty acid is present as a free fatty acid, carboxylate, phospholipid, diacylglycerol, or triacylglycerol). A reductase gene (e.g., an unmodified reductase gene or recombinant reductase gene) may comprise any one of the naturally-occurring nucleotide sequences set forth in SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:100, SEQ ID NO:106, SEQ ID NO:110, SEQ ID NO:114, SEQ ID NO:118, or SEQ ID NO:122. A reductase may be a 10-methylstearic A gene (tmsA) as described herein, or a biologically-active portion thereof (i.e., wherein the biologically-active portion thereof comprises reductase activity.

A reductase gene may be derived from a gram-positive species of Actinobacteria, such as Bacillus, Haemophilus, Vibrio harvevi, Rhodobacter, Escherichia, Staphylococci, Streptomycete, or Corynebacteria. A reductase gene may be selected from the group consisting of Mycobacterium smegmatis gene tmsA, Agromyces subbeticus gene tmsA, Amycolicicoccus subflavus gene tmsA, Corynebacterium glutamicum gene tmsA, Corynebacterium glyciniphilium gene tmsA, Knoella aerolata gene tmsA, Mycobacterium austroafricanum gene tmsA, Mycobacterium gilvum gene tmsA, Mycobacterium indices pranii gene tmsA, Mycobacterium phlei gene tmsA, Mycobacterium tuberculosis gene tmsA, Mycobacterium vanbaalenii gene tmsA, Rhodococcus opacus gene tmsA, Streptomyces regnsis gene tmsA, Thermobifida fusca gene tmsA, and Thermomonospora curvata gene tmsA.

A recombinant reductase gene may be recombinant because it is operably-linked to a promoter other than the naturally-occurring promoter of the reductase gene. Such genes may be useful to drive transcription in a particular species of cell. A recombinant reductase gene may be recombinant because it contains one or more nucleotide substitutions relative to a naturally-occurring reductase gene. Such genes may be useful to increase the translation efficiency of the reductase gene's mRNA transcript in a particular species of cell.

A nucleic acid may comprise a reductase gene and a promoter, wherein the reductase gene and promoter are operably-linked. The reductase gene and promoter may be derived from different species. For example, the reductase gene may encode the reductase protein of a gram-positive species of Actinobacteria, and the reductase gene may be operably-linked to a promoter that can drive transcription in another phylum of bacteria a Proteobacterium, such as E. coli) or a eukaryote (e.g., an algae cell, yeast cell, or plant cell). The promoter may be a eukaryotic promoter. A cell may comprise the nucleic acid, and the promoter may be capable of driving transcription in the cell. A cell may comprise a reductase gene, and the reductase gene may be operably-linked to a promoter capable of driving transcription of the reductase gene in the cell. The cell may be a species of yeast, and the promoter may be a yeast promoter. The cell may be a species of bacteria, and the promoter may be a bacterial promoter (e.g., wherein the bacterial promoter is not a promoter from Actinobacteria). The cell may be a species of algae, and the promoter may be an algae promoter. The cell may be a species of plant, and the promoter may be a plant promoter.

A reductase gene may be operably-linked to a promoter that cannot drive transcription in the cell from which the reductase gene originated. For example, the promoter may not be capable of binding an RNA polymerase of the cell from which a reductase gene originated. In some embodiments, the promoter cannot bind a prokaryotic RNA polymerase and/or initiate transcription mediated by a prokaryotic RNA polymerase. In some embodiments, a reductase gene is operably-linked to a promoter that cannot drive transcription in the cell from which the protein encoded by the gene originated. For example, the promoter may not be capable of binding an RNA polymerase of a cell that naturally expresses the reductase enzyme encoded by a reductase gene.

A promoter may be an inducible promoter or a constitutive promoter. A promoter may be any one of the promoters described in PCT Patent Application Publication No. WO 2016/014900, published Jan. 28, 2016 (hereby incorporated by reference in its entirety). WO 2016/014900 describes various promoters derived from yeast species Yarrowia lipolytica and Arxula adeninivorans, which may be particularly useful as promoters for driving the transcription of a recombinant gene in a yeast cell. A promoter may be a promoter from a gene encoding a Translation Elongation factor LF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1, Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pᵢ cotransporter; Pyruvate decarboxylase; Phytase; or Alpha-amylase, e.g., wherein the gene is a yeast gene, such as a gene from Yarrowia lipolytica or Arxula adeninivorans.

A reductase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:100, SEQ ID NO:106, SEQ ID NO:110, SEQ ID NO:114, SEQ ID NO:118, or SEQ ID NO:122. A reductase gene may comprise a nucleotide sequence with, with at least, with at most 65%, 70%, 75%, 80%, 85%, 90%, 95%, 96%, 97%, 98%, or 99% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:100, SEQ ID NO:106, SEQ ID NO:110, SEQ ID NO:114, SEQ ID NO:118, or SEQ ID NO:122. A reductase may or may not have 100% sequence identity with any one of the nucleotide sequences set forth in SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:100, SEQ ID NO:106, SEQ ID NO:110, SEQ ID NO:114, SEQ ID NO:118, or SEQ ID NO:122. A reductase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:100, SEQ ID NO:106, SEQ ID NO:110, SEQ ID NO:114, SEQ ID NO:118, or SEQ ID NO:122. A reductase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, 99%, or 100% sequence identity with the nucleotide sequence set forth in SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, and SEQ ID NO:65, and the reductase gene may encode a reductase protein with at least about 95%, 96%, 97%, 98%, 99%, or 100% sequence identity with the amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO: 115, SEQ ID NO: 119, or SEQ ID NO: 123. For example, SEQ NO:34 is a gene for expression in yeast. SEQ ID NO:34 does not have 100% sequence identity with SEQ ID NO:36, and the protein encoded by SEQ ID NO:34 has at least about 99% sequence identity with the amino acid sequence set forth in SEQ ID NO:36.

A recombinant reductase gene may vary from a naturally-occurring reductase gene because the recombinant reductase gene may be codon-optimized for expression in a eukaryotic cell, such as a plant cell, algae cell, or yeast cell. A cell may comprise a recombinant reductase gene, wherein the recombinant reductase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant reductase gene may vary from a naturally-occurring reductase gene or may be unchanged from a naturally-occurring reductase gene. For example, a recombinant reductase gene may comprise a nucleotide sequence with at least 65% sequence identity with the naturally-occurring nucleotide sequence set forth in SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:100, SEQ ID NO:106, SEQ ID NO:110, SEQ ID NO:114, SEQ ID NO:118, or SEQ ID NO:122 (e.g., at least 70%, 75%, 80%, 85%, 90%, 95%, 96%, 97%, 98%, or 99% sequence identity), and at least 5 codons of the nucleotide sequence of the recombinant reductase gene may vary from the naturally-occurring nucleotide sequence (e.g., at least 10, 15, 20, 25, 30, 35, 40, 45, 50, 60, 70, 80, 90, or 100 codons).

A reductase gene encodes a reductase protein. A reductase protein may be a protein expressed by a gram-positive species of Actinobacteria, such as Mycobacteria, Corynebacteria, Nocardia, Streptomyces, or Rhodococcus. A recombinant reductase gene may encode a naturally-occurring reductase protein even if the recombinant reductase gene is not a naturally-occurring reductase gene. For example, a recombinant reductase gene may vary from a naturally-occurring reductase gene because the recombinant reductase gene is codon-optimized for expression in a specific cell. The codon-optimized, recombinant reductase gene and the naturally-occurring reductase gene may nevertheless encode the same naturally-occurring reductase protein.

A reductase gene may encode a reductase protein selected from Mycobacterium smegmatis enzyme TmsA, Agromyces subbeticus enzyme TmsA, Amycolicicoccus subflavus enzyme TmsA, Corynebacterium glutamicum enzyme TmsA, Corynebacterium glyciniphilium enzyme TmsA, Knoella aerolata enzyme TmsA, Mycobacterium austroafricanum enzyme TmsA, Mycobacterium gilvum enzyme TmsA, Mycobacterium indicus pranii enzyme TmsA, Mycobacterium phlei enzyme TmsA, Mycobacterium tuberculosis enzyme TmsA, Mycobacterium vanbaalenii enzyme TmsA, Rhodococcus opacus enzyme TmsA, Streptomyces regnsis enzyme TmsA, Thermobifida fusca enzyme TmsA, and Thermomonospora curvata enzyme TmsA. A reductase gene may encode a reductase protein, and the reductase protein may be substantially identical to any one of the foregoing enzymes, but the recombinant reductase gene may vary from the naturally-occurring gene that encodes the enzyme. The recombinant reductase gene may vary from the naturally-occurring gene because the recombinant reductase gene may be codon-optimized for expression in a specific phylum, class, order, family, genus, species, or strain of cell.

The sequences of naturally-occurring reductase proteins are set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO:115, SEQ ID NO: 119, or SEQ ID NO:123. A recombinant reductase gene may or may not encode a protein comprising 100% sequence identity with the amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO:115, SEQ ID NO: 119, or SEQ ID NO:123. For example, a recombinant reductase gene may encode a protein having 100% sequence identity with a biologically-active portion of an amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO:115, SEQ ID NO: 119, or SEQ ID NO:123.

A recombinant reductase gene may encode a reductase protein having, having at least, or having at most 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93% 94% 95%, 96%, 97%, 98%, 99%, or 100% sequence identity with the amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO:115, SEQ ID NO: 119, or SEQ ID NO:123, or a biologically-active portion thereof. A recombinant reductase gene may encode a reductase protein having about, at least about, or at most about 0.1%, 0.5%, 1%, 2%, 3%, 4%, 5%, 10%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%; 280%, 300%, 320%, 340%, 360%, 380%, or 400% reductase activity relative to a protein comprising the amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO:115, SEQ ID NO: 119, or SEQ ID NO:123. A recombinant reductase gene may encode a protein having, having at least, or having at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% 94%, 95%, 96%, 97%, 98%, 99%, or 100% sequence identity with 10, 20, 30, 40, 50, 60, 70, 80, 90, 100, 110, 120, 130, 140; 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous amino acids starting at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206; 207, 208, 209, 210, 211, 212, 213, 214, 215, 216, 217, 218, 219, 220, 221, 222, 223, 224, 225, 226, 227, 228, 229; 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 of the amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO:115, SEQ ID NO: 119, or SEQ ID NO:123.

Substrates for the reductase protein may include any fatty acid from 12 to 20 carbons long with a methylene substitution in the 7, 8, 9, 10, 11, or 12 position. The fatty acid substrate may be 12, 13, 14, 15, 16, 17, 18, 19, or 20 carbons long, or any range derivable therein. The reductase protein may be capable of catalyzing the reduction of a methylene-substituted fatty acid substrate to a (methyl)lipid. The reductase protein, together with a methyltransferase protein, may be capable of catalyzing the production of a methylated branch from any fatty acid from 12 to 20 carbons long with an unsaturated double bond in the Δ7, Δ8, Δ9, Δ10, or Δ11 position.

In some embodiments, the reductase gene encodes a reductase protein that includes a flavin adenine dinucleotide (FAD) binding domain. In some embodiments, the FAD binding domain has at least 80%, 85%, 90%, 91%, 92%, 93%, 94%, 95%, 96%, 97%, 98%, 99%, 99.9%, or 100% sequence identity to amino acids 9-141 of T. curvata TmsA (SEQ ID NO:74) or to a corresponding portion of TmsA from Mycobacterium smegmatis, Mycobacterium vanbaaleni, Amycolicicoccus subflavus, Corynebacterium glyciniphilium, Corynebacterium glutamicum, Rhodococcus opacus, Agromyces subbeticus, Knoellia aerolata, Mycobacterium gilvum, Mycobacterium sp. Indicus, or Thermobifida fusca.

In some embodiments, the reductase gene encodes a reductase protein that includes a FAD/FMN-containing dehydrogenase domain. In some embodiments, the FAD/FMN-containing dehydrogenase domain has, has at least, or has at most 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to amino acids 22-444 of T. curvata TmsA (SEQ ID NO:64) or to a corresponding portion of TmsA from Mycobacterium smegmatis, Mycobacterium vanbaaleni, Amycolicicoccus subflavus, Corynebacterium glyciniphilium, Corynebacterium glutamicum, Rhodococcus opacus, Agromyces subbeticus, Knoellia aerolata, Mycobacterium gilvum, Mycobacterium sp. Indicus, or Thermobifida fusca.

In some embodiments, the reductase gene encodes a reductase protein that has specific amino acids unchanged from the amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO:115, SEQ ID NO: 119, or SEQ ID NO:123. The unchanged amino acids can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or amino acids selected from R31, A33, S37, N38, L39, F40, R43, D52, V59, D63, G73, M74, T76, Y77, D79, L80, V81, L85, P91, V93, V94, Q96, L97, T99, I100, T101, A105, G108, G110, E112, S113, S115, F116, R117, N118, P121, H122, E123, V125, E127, G133, P154, N155, Y157, Y162, L166, E171, V173, V177, H181, V208, G213, F216, Y222, L223, S236, D237, Y238, T239, Y245, S247, D254, T257, Y261, W263, R264, W265, D266, D268, W269, C272, A275, G277, Q279, R284, W287, R293, S294, G318, E232, V325, P328, E330, F339, F343, W353, C355, P356, W363, L365, Y366, P367, N376, F379, W380, V383, P384, N395, E399, G407, H408, K409, S410, L411, Y412, S413, Y417, F422, Y426, G428, R443, L447, and V452 of T. curvata TmsA (SEQ ID NO:74) or corresponding amino acids in TmsA from Mycobacterium smegmatis, Mycobacterium vanbaaleni, Amycolicicoccus subflavus, Corynebacterium glyciniphilium, Corynebacterium glutamicum, Rhodococcus opacus, Agromyces subbeticus, Knoellia aerolata, Mycobacterium gilvum, Mycobacterium sp. Indicus, or Thermobifida fusca.

iii. Nucleic Acids Comprising a tmsC Gene (e.g., Recombinant tmsC Gene).

A nucleic acid may comprise a 10-methylstearic C gene (tmsC), as described herein. A tmsC gene (e.g., a recombinant tmsC gene) may comprise any one of the nucleotide sequences set forth in SEQ ID NO:77, SEQ ID NO:67, SEQ ID NO:69, SEQ ID NO:71, SEQ ID NO:73, SEQ ID NO:75, SEQ ID NO: 128, SEQ ID NO:130, SEQ ID NO:132, or SEQ ID NO:134. A tmsC gene (e.g., a recombinant tmsC gene) may be derived from a gram-positive species of Actinobacteria, such as Mycobacteria, Corynebacteria, Nocardia, Streptomyces, or Rhodococcus. A tmsC gene (e.g., a recombinant tmsC gene) may be selected from the group consisting of Corynebacterium glyciniphilium gene time, Mycobacterium austroafricanum gene tmsC, Mycobacterium gilvum gene tmsC, Mycobacterium vanbaalenii gene tmsC, Streptomyces regnsis gene tmsC, and Thermobifida fusca gene tmsC.

A recombinant tmsC gene may be recombinant because it is operably-linked to a promoter other than the naturally-occurring promoter of the tmsC gene. Such genes may be useful to drive transcription in a particular species of cell. A recombinant tmsC gene may be recombinant because it contains one or more nucleotide substitutions relative to a naturally-occurring tmsC gene. Such genes may be useful to increase the translation efficiency of the tmsC gene's mRNA transcript in a particular species of cell.

A nucleic acid may comprise a recombinant tmsC gene and a promoter, wherein the recombinant tmsC gene and promoter are operably-linked. The tmsC gene and promoter may be derived from different species. For example, the tmsC gene may encode the TmsC protein of a gram-positive species of Actinobacteria, and the tmsC gene may be operably-linked to a promoter that can drive transcription in another phylum of bacteria (e.g., a Proteobacterium, such as E. coli) or a eukaryote (e.g., an algae cell, yeast cell, or plant cell). The promoter may be a eukaryotic promoter. A cell may comprise the nucleic acid, and the promoter may be capable of driving transcription in the cell. A cell may comprise a recombinant tmsC gene, and the recombinant tmsC gene may be operably-linked to a promoter capable of driving transcription of the recombinant tmsC gene in the cell. The cell may be a species of yeast, and the promoter may be a yeast promoter. The cell may be a species of bacteria, and the promoter may be a bacterial promoter (e.g., wherein the bacterial promoter is not a promoter from Actinobacteria). The cell may be a species of algae, and the promoter may be an algae promoter. The cell may be a species of plant; and the promoter may be a plant promoter.

A recombinant tmsC gene may be operably-linked to a promoter that cannot drive transcription in the cell from which the recombinant tmsC gene originated. For example, the promoter may not be capable of binding an RNA polymerase of the cell from which a recombinant tmsC gene originated. In some embodiments, the promoter cannot bind a prokaryotic RNA polymerase and/or initiate transcription mediated by a prokaryotic RNA polymerase. In some embodiments, a recombinant tmsC gene is operably-linked to a promoter that cannot drive transcription in the cell from which the protein encoded by the gene originated. For example, the promoter may not be capable of binding an RNA polymerase of a cell that naturally expresses the TmsC enzyme encoded by a recombinant tmsC gene.

A promoter may be an inducible promoter or a constitutive promoter. A promoter may be any one of the promoters described in PCT Patent Application Publication No. WO 2016/014900, published Jan. 28, 2016 (hereby incorporated by reference in its entirety). WO 2016/014900 describes various promoters derived from yeast species Yarrowia lipolytica and Arxula adeninivorans, which may be particularly useful as promoters for driving the transcription of a recombinant gene in a yeast cell. A promoter may be a promoter from a gene encoding a Translation Elongation factor EF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1; Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pᵢ cotransporter; Pyruvate decarboxylase; Phytase; or Alpha-amylase, e.g., wherein the gene is a yeast gene, such as a gene from Yarrowia lipolytica or Arxula adeninivorans.

A recombinant tmsC gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% 94% 95%, 96%, 97%, 98%, 99%, or 100% sequence identity with the nucleotide sequence set forth in SEQ ID NO:77, SEQ ID NO:67, SEQ ID NO:69, SEQ ID NO:71, SEQ ID NO:73, SEQ ID NO:75, SEQ ID NO:128, SEQ ID NO:130, SEQ ID NO:132, or SEQ ID NO:134. A tmsC may or may not have 100% sequence identity with any one of the nucleotide sequences set forth in SEQ ID NO:77, SEQ ID NO:67, SEQ ID NO:69, SEQ ID NO:71, SEQ ID NO:73, SEQ ID NO:75, SEQ ID NO:128, SEQ ID NO:130, SEQ ID NO:132, or SEQ ID NO:134. A tmsC gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:77, SEQ ID NO:67, SEQ ID NO:69, SEQ ID NO:71, SEQ ID NO:73, SEQ ID NO:75, SEQ ID NO:128, SEQ ID NO:130, SEQ ID NO:132, or SEQ ID NO:134, and the tmsC gene may encode a TmsC protein with at least about 95%, 96%, 97%, 98%, 99%, or 100% sequence identity with the amino acid sequence set forth in SEQ ID NO:66, SEQ ID NO:68, SEQ ID NO:70, SEQ ID NO:72, SEQ ID NO:74, SEQ ID NO:76, SEQ ID NO:129, SEQ ID NO:131, SEQ ID NO:133, or SEQ ID NO:135.

A recombinant tmsC gene may vary from a naturally-occurring tmsC gene because the recombinant tmsC gene may be codon-optimized for expression in a eukaryotic cell, such as a plant cell, algae cell, or yeast cell. A cell may comprise a recombinant tmsC gene, wherein the recombinant tmsC gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant tmsC gene may vary from a naturally-occurring tmsC gene or may remain unchanged from a naturally-occurring tmsC gene. For example, a recombinant tmsC gene may comprise a nucleotide sequence with at least about 65% sequence identity with the naturally-occurring nucleotide sequence set forth in SEQ ID NO:77, SEQ ID NO:67, SEQ ID NO:69, SEQ ID NO:71, SEQ ID NO:73, SEQ ID NO:75, SEQ ID NO:128, SEQ ID NO:130, SEQ ID NO:132, or SEQ ID NO:134 (e.g., at least about 70%, 75%, 80%, 85%, 90%, 95%, 96%, 97%, 98%, or 99% sequence identity), and at least 5 codons of the nucleotide sequence of the recombinant tmsC gene may vary from the naturally-occurring nucleotide sequence (e.g., at least about 10, 15, 20, 25, 30, 35, 40, 45, 50, 60, 70, 80, 90, or 100 codons).

A tmsC gene encodes a TmsC protein. A TmsC protein may be a protein expressed by a gram-positive species of Actinobacteria, such as Mycobacteria, Corynebacteria, Nocardia, Streptomyces, or Rhodococcus. A recombinant tmsC gene may encode a naturally-occurring TmsC protein even if the recombinant tmsC gene is not a naturally-occurring tmsC gene. For example, a recombinant tmsC gene may vary from a naturally-occurring tmsC gene because the recombinant tmsC gene is codon-optimized for expression in a specific cell. The codon-optimized, recombinant tmsC gene and the naturally-occurring tmsC gene may nevertheless encode the same naturally-occurring TmsC protein.

A recombinant tmsC gene may encode a TmsC protein selected from Corynebacterium glyciniphilium enzyme TmsC, Mycobacterium austroafricanum enzyme TmsC, Mycobacterium gilvum enzyme TmsC, Mycobacterium vanbaalenii enzyme TmsC, Streptomyces regnsis enzyme TmsC, and Thermobifida fusca enzyme TmsC. A recombinant tmsC gene may encode a TmsC protein, and the TmsC protein may be substantially identical to any one of the foregoing enzymes, but the recombinant tmsC gene may vary from the naturally-occurring gene that encodes the enzyme. The recombinant tmsC gene may vary from the naturally-occurring gene because the recombinant tmsC gene may be codon-optimized for expression in a specific phylum, class, order, family, genus, species, or strain of cell.

The sequences of naturally-occurring TmsC proteins are set forth in SEQ ID NO:66, SEQ ID NO:68, SEQ ID NO:70, SEQ ID NO:72, SEQ ID NO:74, SEQ ID NO:76, SEQ ID NO:129, SEQ ID NO:131, SEQ ID NO:133, or SEQ ID NO:135. A recombinant tmsC gene may or may not encode a protein comprising 100%, sequence identity with the amino acid sequence set forth in SEQ ID NO:66, SEQ ID NO:68, SEQ ID NO:70, SEQ ID NO:72, SEQ ID NO:74, and SEQ ID NO:76. For example, a recombinant tmsC gene may encode a protein having 100% sequence identity with a biologically-active portion of an amino acid sequence set forth in SEQ ID NO:66, SEQ ID NO:68, SEQ ID NO:70, SEQ ID NO:72, SEQ ID NO:74, SEQ ID NO:76, SEQ ID NO:129, SEQ ID NO:131, SEQ ID NO:133, or SEQ ID NO:135. A recombinant tmsC gene may encode a TmsC protein having at least about 95%, 96%, 97%, 98%, or 99% sequence identity with the amino acid sequence set forth in SEQ ID NO:66, SEQ ID NO:68, SEQ ID NO:70, SEQ ID NO:72, SEQ ID NO:74, SEQ ID NO:76, SEQ ID NO:129, SEQ ID NO:131, SEQ ID NO:133, or SEQ ID NO:135, or a biologically-active portion thereof.

iv. Nucleic Acids Comprising a Methyltransferase Gene and a Reductase Gene

A nucleic acid may comprise both a methyltransferase gene (recombinant or unmodified) and a reductase gene (recombinant or unmodified). The methyltransferase gene and the reductase gene may encode proteins from the same species or from different species. A nucleic acid may comprise a methyltransferase gene, a reductase gene, and/or a tmsC gene. A methyltransferase gene, reductase gene, and a tmsC gene may encode proteins from 1, 2, or 3 different species (i.e., the genes may each be from the same species, two genes may be from the same species, or all three genes may be from different species).

A nucleic acid may comprise the nucleotide sequence set forth in SEQ ID NO:91, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:128, SEQ ID NO:130, SEQ ID NO:132, or SEQ ID NO:134. A nucleic acid may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in SEQ ID NO:91, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:128, SEQ ID NO:130, SEQ ID NO:132, or SEQ ID NO:134.

In some embodiments, the nucleic acid encodes a fusion protein that includes both a methyltransferase and a reductase or fragments thereof. In the context of the present disclosure, “fusion protein” means a single protein molecule containing two or more distinct proteins or fragments thereof, covalently linked via peptide bond in a single peptide chain. In some embodiments, the fusion protein comprises enzymatically active domains from both a methyltransferase protein and a reductase protein. The nucleic acid may further encode a linker peptide between the methyltransferase and the reductase. In some embodiments, the linker peptide comprises the amino acid sequence AGGAEGGNGGGA. The linker may comprise about or at least about 2, 3, 4, 5, 6, 7, 9, 10, 15, 20, 25, or 30 amino acids, or any range derivable therein. The nucleic acid may comprise any of the methyltransferase and reductase genes described herein, and the fusion protein encoded by the nucleic acid can comprise any of the methyltransferase and reductase proteins described herein, including biologically active fragments thereof. In some embodiments, the fusion protein is a tmsA-B protein, in which the TmsA protein is closer to the N-terminus than the TmsB protein. An example of such a TmsA-B protein is encoded by the nucleic acid sequence of SEQ ID NO:90. In some embodiments, the fusion protein is a TmsB-A protein, in which the TmsB protein is closer to the N-terminus than the TmsA protein. An example of such a TmsB-A protein is encoded by the nucleic acid sequence of SEQ ID NO:93. In some embodiments, the fusion protein has at least about 80%, 85%, 90%, 91%, 92%, 93%, 94%, 95%, 96%, 97%, 98%, 99%, or 99.9% identity to the amino acid sequence of a fusion protein encoded by SEQ ID NO:90 or SEQ ID NO:93.

Exemplary Cells, Nucleic Acids, Compositions, and Methods for Terminal Fatty Acid Alkylation

Alternatively or in addition to internal fatty acid methylation described above, methods of the present disclosure can include terminal fatty acid alkylation.

Terminal fatty acid alkylation can be performed using one or more alkyl transferases, such as a β-ketoacyl-acyl carrier protein synthase. As used herein, “alkyl transferase” is used interchangeably with “terminal alkyl transferase” and may include, but is not limited to, terminal methyl transferases and/or terminal ethyl transferases. As used herein, the term “terminal” includes, but is not limited to, the three carbon atoms located at the terminus along a fatty acid chain.

As used herein, “terminal fatty acid alkylation” can include alkylation (e.g., methylation and/or ethylation) of one or more of: (1) aterminal carbon atom, (2) carbon atom alpha to the terminal carbon atom, or (3) carbon atom beta to the terminal carbon atom of a fatty acid. Terminal fatty acid alkylation may be performed in the same bioreactor as the internal fatty acid methylation or may be performed in a separate bioreactor as the internal fatty acid methylation.

Alkyl transferases (e.g., β-ketoacyl-acyl carrier protein synthases), for example, are utilized in fatty acid biosynthesis (FAB). FAB is utilized for the production of bacterial cell walls, and therefore is essential for the survival of bacteria (Magnuson et al., 1993, Microbiol. Rev. 57:522-542). The fatty acid synthase system in E. coli, for example, is an exemplary type II fatty acid synthase system. Multiple enzymes are involved in fatty acid biosynthesis, and genes encoding the enzymes FabH, FabD, FabG, AcpP, and FabF are clustered together on the E. coli chromosome. Clusters of FAB genes have also been found in Bacillus subtilis, Staphylococcus aureus, Haemophilus influenza Rd, Vibrio harveyi, and Rhodobacter capsulatus.

An alkyl transferase gene (e.g., an unmodified reductase gene or recombinant reductase gene) encodes an alkyl transferase protein, which is an enzyme capable of alkylating a terminal carbon of a fatty acid (e.g., wherein the fatty acid is present as a free fatty acid, carboxylate, phospholipid, diacylglycerol, or triacylglycerol). For example, fatty acid synthesis can be initiated by the condensation of acetyl-coenzyme A (acetyl-COA) with malonyl-acyl carrier protein (malonyl-ACP) by β-ketoacyl-acyl carrier protein synthase III, the product of the fabH gene.

An alkyl transferase gene (e.g., an unmodified alkyl transferase gene or recombinant alkyl transferase gene) may comprise any one of the naturally-occurring nucleotide sequences set forth in SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, or SEQ ID NO:160. An alkyl transferase may be a s-ketoacyl-acyl carrier protein synthase gene as described herein, or a biologically-active portion thereof (i.e., wherein the biologically-active portion thereof comprises alkyl transferase activity.

An alkyl transferase gene may be derived from any host cell suitable for expression of an alkyl transferase gene, such as fungal or yeast species, such as Arxula, Aspergillus, Aurantiochytrium, Candida, Claviceps, Cryptococcus, Cunninghamella, Hansenula, Kluyveromyces, Leucosporidiella, Lipomyces, Mortierella, Ogataea, Pichia, Prototheca, Rhizopus, Rhodosporidium, Rhodotorula, Saccharomyces, Schizosaccharomyces, Tremella, Trichosporon, Yarrowia, or bacterial species, such as members of proteobacteria and actinomycetes, as well as the genera Acinetobacter, Arthrobacter, Brevibacterium, Acidovorax, Bacillus, Clostridia, Streptomyces, Escherichia, Staphylococci, Streptomycete, Rickettsia prowazekii, Clamydia trachomatis, Aquifex aeolicus, Helicobacter pylori, Haemophilus influenzae, Salmonella, Pseudomonas, and Cornyebacterium. Yarrowia lipolytica and Arxula adeninivorans are suited for use as a host microorganism because they can accumulate a large percentage of their weight as triacylglycerols.

For example, an alkyl transferase gene may be derived from a gram-negative species of Proteobacterium, such as Escherichia, such as Escherichia coli. An alkyl transferase gene may be selected from the group consisting of Escherichia Coli gene eFabH and Escherichia Coli gene fabH.

An alkyl transferase gene may be derived from a gram-positive species of Firmicute, such as Bacillus, such as Bacillus subtilis. An alkyl transferase gene may be selected from the group consisting of Bacillus subtilis gene bFabH1 and Bacillus subtilis gene bFabH2.

An alkyl transferase gene may be derived from a gram-positive species of Streptomyces, such as Streptomyces glaucescens. An alkyl transferase gene may be Streptomyces glaucescens gene eFabH.

An alkyl transferase gene may be native or the alkyl transferase may be recombinant because it is operably-linked to a promoter other than the naturally-occurring promoter of the alkyl transferase gene. Such genes may be useful to drive transcription in a particular species of cell. A recombinant alkyl transferase gene may be recombinant because it contains one or more nucleotide substitutions relative to a naturally-occurring alkyl transferase gene. Such genes may be useful to increase the translation efficiency of the alkyl transferase gene's mRNA transcript in a particular species of cell.

A nucleic acid may comprise an alkyl transferase gene and a promoter, wherein the alkyl transferase gene and promoter are operably-linked. The alkyl transferase gene and promoter may be derived from different species. For example, the alkyl transferase gene may encode the alkyl transferase protein of a gram-negative species of Proteobacterium or gram-positive species of Firmicute, and the alkyl transferase gene may be operably-linked to a promoter that can drive transcription in another phylum of bacteria a (gram-positive species of Actinobacteria) or a eukaryote (e.g., an algae cell, yeast cell, or plant cell). The promoter may be a eukaryotic promoter. A cell may comprise the nucleic acid, and the promoter may be capable of driving transcription in the cell. A cell may comprise an alkyl transferase gene, and may be operably-linked to a promoter capable of driving transcription of the alkyl transferase gene in the cell. The cell may be a species of yeast, and the promoter may be a yeast promoter. The cell may be a species of bacteria, and the promoter may be a bacterial promoter (e.g., wherein the bacterial promoter is not a promoter from Proteobacterium). The cell may be a species of algae, and the promoter may be an algae promoter. The cell may be a species of plant, and the promoter may be a plant promoter.

An alkyl transferase gene may be operably-linked to a promoter that cannot drive transcription in the cell from which the alkyl transferase gene originated. For example, the promoter may not be capable of binding an RNA polymerase of the cell from which an alkyl transferase gene originated. In some embodiments, the promoter cannot bind a prokaryotic RNA polymerase and/or initiate transcription mediated by a prokaryotic RNA polymerase. In some embodiments, an alkyl transferase gene is operably-linked to a promoter that cannot drive transcription in the cell from which the protein encoded by the gene originated. For example, the promoter may not be capable of binding an RNA polymerase of a cell that naturally expresses the alkyl transferase enzyme encoded by an alkyl transferase gene.

A promoter may be an inducible promoter or a constitutive promoter. A promoter may be any one of the promoters described in PCT Patent Application Publication No. WO 2016/014900, published Jan. 28, 2016 (hereby incorporated by reference in its entirety). WO 2016/014900 describes various promoters derived from yeast species Yarrowia lipolytica and Arxula adeninivorans, which may be particularly useful as promoters for driving the transcription of a recombinant gene in a yeast cell. A promoter may be a promoter from a gene encoding a Translation Elongation factor LF-1α; Glycerol-3-phosphate dehydrogenase; Triosephosphate isomerase 1; Fructose-1,6-bisphosphate aldolase; Phosphoglycerate mutase; Pyruvate kinase; Export protein EXP1, Ribosomal protein S7; Alcohol dehydrogenase; Phosphoglycerate kinase; Hexose Transporter; General amino acid permease; Serine protease; Isocitrate lyase; Acyl-CoA oxidase; ATP-sulfurylase; Hexokinase; 3-phosphoglycerate dehydrogenase; Pyruvate Dehydrogenase Alpha subunit; Pyruvate Dehydrogenase Beta subunit; Aconitase; Enolase; Actin; Multidrug resistance protein (ABC-transporter); Ubiquitin; GTPase; Plasma membrane Na+/Pᵢ cotransporter; Pyruvate decarboxylase; Phytase; or Alpha-amylase, e.g., wherein the gene is a yeast gene, such as a gene from Yarrowia lipolytica or Arxula adeninivorans.

An alkyl transferase may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in the naturally-occurring nucleotide sequences set forth in SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, or SEQ ID NO:160. An alkyl transferase gene may comprise a nucleotide sequence with, with at least, with at most 65%, 70%, 75%, 80%, 85%, 90%, 95%, 96%, 97%, 98%, or 99% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, or SEQ ID NO:160. An alkyl transferase gene may or may not have 100% sequence identity with any one of the nucleotide sequences set forth in nucleotide sequences set forth in SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, or SEQ ID NO:160. An alkyl transferase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, or SEQ ID NO:160. An alkyl transferase gene may comprise a nucleotide sequence with, with at least, or with at most 65%, 66%, 67%, 68%, 69%, 70%, 71%, 72%, 73%, 74%, 75%, 76%, 77%, 7⁸%, 79%, 80%, 81%, 82%, 83%, 84%, 85%, 86%, 87%, 88%, 89% 90%, 91%, 92%, 93%, 94%, 95%, 96%, 97%, 98%, 99%, or 100% sequence identity with the nucleotide sequence set forth in SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, or SEQ ID NO:160, and the alkyl transferase gene may encode an alkyl transferase protein with at least about 95%, 96%, 97%, 98%, 99%, or 100% sequence identity with the amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161. For example, the protein encoded by SEQ ID NO:136 does not have 100% sequence identity with the amino acid sequence set forth in SEQ ID NO:161.

A recombinant alkyl transferase gene may vary from a naturally-occurring alkyl transferase gene because the recombinant alkyl transferase gene may be codon-optimized for expression in a eukaryotic cell, such as a plant cell, algae cell, or yeast cell. A cell may comprise a recombinant alkyl transferase gene, wherein the recombinant alkyl transferase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant alkyl transferase gene may vary from a naturally-occurring alkyl transferase gene or may be unchanged from a naturally-occurring alkyl transferase gene. For example, a recombinant alkyl transferase gene may comprise a nucleotide sequence with at least 65% sequence identity with the naturally-occurring nucleotide sequence set forth in SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, or SEQ ID NO:160, (e.g., at least 70%, 75%, 80%, 85%, 90%, 95%, 96%, 97%, 98%, or 99% sequence identity), and at least 5 codons of the nucleotide sequence of the recombinant alkyl transferase gene may vary from the naturally-occurring nucleotide sequence (e.g., at least 10, 15, 20, 25, 30, 35, 40, 45, 50, 60, 70, 80, 90, or 100 codons).

An alkyl transferase gene encodes an alkyl transferase protein. An alkyl transferase protein may be a protein expressed by a gram-positive species of Firmicute or a gram-negative species such as Proteobacterium. A recombinant alkyl transferase gene may encode a naturally-occurring alkyl transferase protein even if the recombinant reductase gene is not a naturally-occurring reductase gene. For example, a recombinant alkyl transferase gene may vary from a naturally-occurring alkyl transferase gene because the recombinant alkyl transferase gene is codon-optimized for expression in a specific cell. The codon-optimized, recombinant alkyl transferase gene and the naturally-occurring alkyl transferase gene may nevertheless encode the same naturally-occurring alkyl transferase protein.

An alkyl transferase gene may encode an alkyl transferase protein, and the alkyl transferase protein may be substantially identical to any one of the foregoing enzymes, but the recombinant alkyl transferase gene may vary from the naturally-occurring gene that encodes the enzyme. The recombinant alkyl transferase gene may vary from the naturally-occurring gene because the recombinant alkyl transferase gene may be codon-optimized for expression in a specific phylum, class, order, family, genus, species, or strain of cell.

The sequences of naturally-occurring alkyl transferase proteins are set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161. A recombinant alkyl transferase gene may or may not encode a protein comprising 100% sequence identity with the amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161. For example, a recombinant alkyl transferase gene may encode a protein having 100% sequence identity with a biologically-active portion of an amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161.

A recombinant alkyl transferase gene may encode an alkyl transferase protein having, having at least, or having at most 65%, 66%, 67%, 68%, 69%, 70%, 71%, 72%, 73%, 74%, 75%, 7⁶%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161, or a biologically-active portion thereof. A recombinant alkyl transferase gene may encode an alkyl transferase protein having about, at least about, or at most about 0.1%, 0.5%, 1%, 2%, 3%, 4%, 5%, 10%, 25%, 30%, 35%, 40%, 45%, 50%, 55%, 60%, 65%, 70%, 75%, 80%, 85%, 86%, 87%, 88%, 89%, 90%, 9l %, 92%, 93%, 94%, 95%, 96%, 97%, 98%, 99%, 99.5%, 99.6%, 99.7%, 99.8%, 99.9%, 100%, 100.1%, 100.2%, 100.3%, 100.4%, 100.5%, 100.6%, 100.7%, 100.8%, 100.9%, 101%, 105%, 110%, 115%, 120%, 125%, 130%, 135%, 140%, 145%, 150%, 160%, 170%, 180%, 190%, 200%, 220%, 240%, 260%; 280%, 300%, 320%, 340%, 360%, 380%, or 400% alkyl transferase activity relative to a protein comprising the amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161. A recombinant alkyl transferase gene may encode a protein having, having at least, or having at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% 94%, 95%, 96%, 97%, 98%, 99%, or 100% sequence identity with 10, 20, 30, 40, 50, 60, 70, 80, 90, 100, 110, 120, 130, 140; 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous amino acids starting at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206; 207, 208, 209, 210, 211, 212, 213, 214, 215, 216, 217, 218, 219, 220, 221, 222, 223, 224, 225, 226, 227, 228, 229; 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 of the amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161.

Substrates for the alkyl transferase protein may include Malonyl-CoA, 2-methylbutyryl-CoA, isovaleryl-CoA, or isobutyryl-CoA, which can then be elongated by reacting with more malonyl-CoA's to make the fatty acid. The initial substrate of the alkyl transferase determines whether the resulting fatty acid is linear (malonyl-CoA), contains a methyl branch at the alpha carbon to the terminal end (isovaleryl-CoA, isobutyryl-CoA), or creates an ethyl branch off the alpha carbon to the terminal end (2-methylbutyryl-CoA, isobutyryl-CoA). SAM methyltransferase/reductase can then react on a completed fatty acid or fatty-ACP. The terminally branched fatty acid/fatty-ACP carbon backbone may be 12, 13, 14, 15, 16, 17, 18, 19, or 20 carbons long, or any range derivable therein.

In some embodiments, the alkyl transferase gene encodes an alkyl transferase protein that includes a FAB binding domain. In some embodiments, the FAB binding domain has at least 80%, 85%, 90%, 91%, 92%, 93%, 94%, 95%, 96%, 97%, 98%, 99%, 99.9%, or 100% sequence identity to amino acids of the binding domain of SEQ ID NO:137 or to a corresponding portion of eFabH from Streptomyces glaucescens.

In some embodiments, the alkyl transferase gene encodes an alkyl transferase protein that has specific amino acids unchanged from the amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161.

### Methods of Producing Alkylated Fatty Acids

Various aspects of the present disclosure relate to methods of producing alkylated fatty acids having a methyl substitution at a carbon atom along the interior of the fatty acid chain (e.g., 7, 8, 9, 10, 11, 12) and one or more alkyl substitutions at a terminal carbon along the fatty acid chain. The methyl substitution at a carbon atom along the interior of the fatty acid chain is performed using a methyl transferase and reductase, and the alkyl substitution at a terminal carbon of the fatty acid is performed using an alkyl transferase. Internal methylation and terminal alkylation may be performed in the same bioreactor or in separate bioreactors (in series). In at least one embodiment, terminal alkylation is performed followed by internal methylation of the fatty acid product(s) of the terminal alkylation.

A method may include methylating a fatty acid with a methyl transferase and reductase by incubating a cell or plurality of cells as described herein, supra, with media. The media may optionally be supplemented with an unbranched, unsaturated fatty acid, such as oleic acid, that serves as a substrate for internal methylation and/or terminal alkylation. Additionally or alternatively, methylating a fatty acid at an internal unsaturated carbon of the fatty acid may be performed by supplementing the media with a fatty acid having a methyl or ethyl substitution at a terminal carbon atom along the fatty acid chain (such as a reaction product of the alkylation product of an alkyl transferase as described herein). The media may optionally be supplemented with methionine or s-adenosyl methionine, which may similarly serve as a substrate. Thus, the method may include contacting a cell or plurality of cells with oleic acid, methionine, or both. The method may include incubating a cell or plurality of cells as described herein, supra, in a bioreactor. The method may comprise recovering lipids from the cells and/or from the culture medium, such as by extraction with an organic solvent.

A method may include degumming the cell or plurality of cells, e.g., to remove proteins. The method may include transesterification or esterification of the lipids of the cells. An alcohol such as methanol or ethanol may be used for transesterification or esterification, e.g., thereby producing a fatty acid methyl ester or fatty acid ethyl ester.

A method includes alkylating a fatty acid at a terminal carbon of the fatty acid using an alkyl transferase by incubating a cell or plurality of cells as described herein, supra, with media. The media may optionally be supplemented with an unbranched, unsaturated fatty acid, such as oleic acid, that serves as a substrate for terminal alkylation. Additionally or alternatively, alkylating a fatty acid at a terminal carbon of the fatty acid may be performed by supplementing the media with a fatty acid having a methyl substitution at a carbon atom along the interior of the fatty acid chain (e.g., 7, 8, 9, 10, 11, 12) (such as a reaction product of the methylated product of a methyl transferase/reductase).

The media may optionally be supplemented with a Coenzyme-A (such as malonyl-CoA, 2-methylbutyryl-CoA, acetyl-CoA, or isovaleryl-CoA). Thus, the method may include contacting a cell or plurality of cells with one or more of ACP, β-mercaptoethanol, NADPH, NADH, urea, glycerol, methionine, thiamine, β-alanine, ampicillin, individual proteins (e.g., bFabH1, bFabH2, etc.), or combination(s) thereof. The method may include incubating a cell or plurality of cells as described herein, supra, in a bioreactor. The method may comprise recovering lipids from the cells and/or from the culture medium, such as by extraction with an organic solvent.

A method may include degumming the cell or plurality of cells, e.g., to remove proteins. The method may include transesterification or esterification of the lipids of the cells. An alcohol such as methanol or ethanol may be used for transesterification or esterification, e.g., thereby producing a fatty acid methyl ester or fatty acid ethyl ester. The method may include hydrolysis of the lipids of the cells to form alkylated free fatty acids (i.e., alkylated fatty acids having a carboxylic acid/carboxyl moiety).

### Bio-Production Reactors (Bioreactors) and Systems

Fermentation systems utilizing methods and/or compositions are also within the scope of the present disclosure.

Any of the microorganisms as described and/or referred to herein may be introduced into an industrial bioreactor (also referred to as a “bio-production system”) where the microorganisms convert a carbon source into a fatty acid or fatty acid derived product in a commercially viable operation. The bio-production system includes the introduction of such a microorganism into a bioreactor vessel, with a carbon source substrate and bio-production media suitable for growing the microorganism, and maintaining the bio-production system within a suitable temperature range (and dissolved oxygen concentration range if the reaction is aerobic or microaerobic) for a suitable time to obtain a desired conversion of a portion of the substrate molecules to a selected chemical product. Industrial bio-production systems and their operation are well-known to those skilled in the arts of chemical engineering and bioprocess engineering.

Bio-productions may be performed under aerobic, microaerobic, or anaerobic conditions, with or without agitation. The operation of cultures and populations of microorganisms to achieve aerobic, microaerobic, and anaerobic conditions are known in the art, and dissolved oxygen levels of a liquid culture comprising a nutrient media and such microorganism populations may be monitored to maintain or confirm a desired aerobic, microaerobic or anaerobic condition.

Any of the microorganisms as described and/or referred to herein may be introduced into an industrial bio-production system where the microorganisms convert a carbon source into a selected chemical product in a commercially viable operation. The bio-production system includes the introduction of such a microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to the selected chemical product.

In various embodiments, components of a medium are provided to a microorganism, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing an embodiment of the biosynthetic pathway(s) taught herein, and the substrates may be combined to form fatty acids substituted with an internal methyl substituent(s) and/or terminal alkyl substituent(s).

Further to types of industrial bio-production, various embodiments of the present disclosure may employ a batch type of industrial bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired microorganism or microorganisms, and bio-production is permitted to occur without adding anything to the system. Typically, however, a “batch” type of bio-production event is batch with respect to the addition of substrate and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate.

A variation on the standard batch system is the fed-batch system. Fed-batch bio-production processes are also suitable for methods of the present disclosure and include a typical batch system with the exception that the nutrients, including the substrate, are added in increments as the bio-production progresses. Fed-batch systems are useful when catabolite repression is apt to inhibit the metabolism of the cells and where it is desirable to have limited amounts of substrate in the media. Measurement of the actual nutrient concentration in fed-batch systems may be measured directly, such as by sample analysis at different times, or estimated on the basis of the changes of measurable factors such as pH, dissolved oxygen and the partial pressure of waste gases such as CO2. Batch and fed-batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. Ollis, McGraw Hill, New York, 1986, herein incorporated by reference for general instruction on bio-production.

Although embodiments of the present disclosure may be performed in batch mode, or in fed-batch mode, it is contemplated that embodiments the present disclosure would be adaptable to continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include a chemostat, wherein fresh media is fed to the vessel while simultaneously removing an equal rate of the vessel contents. The limitation of this approach is that cells are lost and high cell density generally is not achievable. In fact, typically one can obtain much higher cell density with a fed-batch process. Another continuous bioreactor utilizes perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Methods of modulating nutrients and growth factors for continuous bio-production processes as well as techniques for maximizing the rate of product formation are well known in the art of industrial microbiology and a variety of methods are detailed by Brock, supra.

It is contemplated that embodiments of the present disclosure may be practiced using either batch, fed-batch, or continuous processes and that any known mode of bio-production would be suitable. It is contemplated that cells may be immobilized on an inert scaffold as whole cell catalysts and subjected to suitable bio-production conditions for chemical product bio-production, or be cultured in liquid media in a vessel, such as a culture vessel. Thus, embodiments used in such processes, and in bio-production systems using these processes, include a population of microorganisms (e.g., cells (recombinant or unmodified)) of the present disclosure, a culture system comprising such population in a media comprising nutrients for the population, and methods of making a selected chemical product.

Embodiments of the present disclosure include methods of making a selected chemical product in a bio-production system, some of which methods may include obtaining a fatty acid derived product after such bio-production event. For example, a method of making a fatty acid or fatty acid derived product may comprise: providing to a culture vessel a media comprising suitable nutrients; providing to the culture vessel a cell such that the cell produces an alkylated fatty acid having a methyl substitution at a carbon atom along the interior of the fatty acid chain (e.g., 7, 8, 9, 10, 11, 12) and one or more alkyl substitutions at a terminal carbon along the fatty acid chain; and maintaining the culture vessel under suitable conditions for the cell to produce the alkylated fatty acid.

It is within the scope of the present disclosure to produce, and to utilize in bio-production methods and systems, including industrial bio-production systems for production of a fatty acid, a recombinant microorganism genetically engineered to modify one or more aspects effective to increase fatty acid bio-production by at least 20 percent over control microorganism lacking the one or more modifications.

In various embodiments, embodiments are directed to a system for bio-production of an alkylated fatty acid, said system comprising: a fermentation tank suitable for cell culture; a line for discharging contents from the fermentation tank to an extraction and/or separation vessel; and an extraction and/or separation vessel (e.g., a settling tank) suitable for removal of the fatty acid product from cell culture waste. In various embodiments, the system includes one or more pre-fermentation tanks, distillation columns, centrifuge vessels, settling tanks, back extraction columns, mixing vessels, or combinations thereof.

### Fatty Acid Compositions

Various aspects of the present disclosure relate to compositions produced by processes of the present disclosure. A composition may be an oil composition comprised of about or at least about 75%, 80%, 85%, 90%, 95%, or 99% fatty acids.

The composition may comprise alkylated fatty acids having a methyl substitution at a carbon atom along the interior of the fatty acid chain (e.g., 7, 8, 9, 10, 11, 12) and one or more alkyl substitutions at a terminal carbon along the fatty acid chain. The alkylated fatty acid may be a carboxylic acid (e.g., 10,17-dimethylstearic acid; 3-hydroxy-10,17-dimethyloctadecanoic acid; 10,17-dimethylnonadecanoic acid; 3-hydroxy-10,17-dimethylnonadecanoic acid; 10,15-dimethylhexadecanoic acid; 10,15-dimethylheptadecanoic acid), carboxylate (e.g., 10,17-dimethyloctadecanoate; 10,17-dimethylnonadecanoate; 10,15-dimethylhexadecanoate, 10,15-dimethylheptadecanoate), ester (e.g., diacylglycerol, triacylglycerol, phospholipid), thioester (e.g., 10,17-dimethylstearyl CoA, 10,17-dimethylpalmityl CoA, 12,17-dimethyloleoyl CoA, 13,17-dimethyloleoyl CoA, 10,17-dimethyl-octadec-12-enoyl CoA), or amide. The exomethylene-substituted lipid may be a carboxylic acid (e.g., 10-methylenestearic acid, 10-methylenepalmitic acid, 12-methyleneoleic acid, 13-methyleneoleic acid, 10-methylene-octadec-12-enoic acid), carboxylate (e.g., 10-methylenestearate, 10-methylenepalmitate, 12-methyleneoleate, 13-methyleneoleate, 10-methylene-octadec-12-enoate), ester (e.g., methyl 13-methyl-9-methylenetetradecanoate, methyl 7-methylenedodecanoate, methyl 11-methyl-7-methylenedodecanoate, methyl 11-methyl-7-methylenetridecanoate, diacylglycerol, triacylglycerol, phospholipid), thioester (e.g., 10-methylenestearyl CoA, 10-methylenepalmityl CoA, 12-methyleneoleoyl CoA, 13-methyleneoleoyl CoA, 10-methylene-octadec-12-enoyl CoA), amide, 10-methyl lipids, 10-methylene lipids, or terminally alkylated or alkenylated versions thereof.

In some aspects, the composition is produced by cultivating a culture comprising any of the cells described herein and recovering the oil composition from the cell culture. The cells in the culture may contain any of the methyltransferase genes, reductase genes, and/or alkyl transferase genes described herein. The culture medium and conditions can be chosen based on the species of the cell to be cultured and can be optimized to provide for maximal production of the desired lipid profile.

Various methods are known for recovering a composition from a culture of cells. For example, lipids, lipid derivatives, and hydrocarbons can be extracted with a hydrophobic solvent such as hexane. Lipids and lipid derivatives can also be extracted using liquefaction, oil liquefaction, and supercritical CO₂ extraction. The recovery process may include harvesting cultured cells, such as by filtration or centrifugation, lysing cells to create a lysate, and extracting the lipid/hydrocarbon components using a hydrophobic solvent. Recovering a composition from a culture of cells can additionally or alternatively be performed using one or more settling tanks.

In addition to accumulating within cells, the lipids described herein may be secreted by the cells. In that case, a process for recovering the lipid may not involve creating a lysate from the cells, but collecting the secreted lipid from the culture medium. Thus, the compositions described herein may be made by culturing a cell that secretes one of the lipids described herein, such as a linear fatty acid with a chain length of 12-20 carbons with a methyl branch at the 7, 8, 9, 10, or 11, 12 position and/or a methyl or ethyl branch at a terminal carbon (e.g., the 16 or 17 position).

In some embodiments, the oil composition comprises about, at least about, or at most about 1%, 2%, 3%, 4%, 5%, 6%, 7%, 8%, 9%, 10%, 11%, 12%, 11%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47% 48%, 49%, 50%, 51% 52%, 53% 54% 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight of an alkylated fatty acid of the present disclosure. In some embodiments, 10-methyl,17-methyl fatty acids comprise about, at least about, or at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 87%, 83%, 84%, 85%, 86%, 87%, 88%, 89%, 90%, 91%, 92%, 93%, 94%, 95%, 96%, 97%, 98%, or 99% by weight of the fatty acids in the composition, or any range derivable therein.

### Esterification

In some embodiments, a fatty acid (such as an alkylated fatty acid) can be coupled with one or more alcohols to form a fatty acid ester. For example, an esterification can be performed to transform carboxylic acids into fatty acid esters. For example, when the reaction is carried out with 10,17-dimethylstearic acid with methanol to form methyl 10,17-dimethyloctadecanoate.

Fatty acid esters of the present disclosure can be used as starting materials to form alpha-olefins or may be used as lube basestocks.

Fatty acid esters of the present disclosure can include methyl 7-methyldodecanoate; methyl 7,11-dimethyldodecanoate; methyl 7,11-dimethyltridecanoate; methyl 9-methyltetradecanoate; methyl9,13-dimethyltetradecanoate; methyl9,13-dimethylpentadecanoate; methyl 10,17-dimethyloctadecanoate; methyl 3-hydroxy-10,17-dimethyloctadecanoate; methyl 10,17-dimethylnonadecanoate; methyl3-hydroxy-10,17-dimethylnonadecanoate; methyl 10,15-dimethylhexadecanoate; methyl 10,15-dimethylheptadecanoate; ethyl 10,17-dimethyloctadecanoate; ethyl 3-hydroxy-10,17-dimethyloctadecanoate; ethyl 10,17-dimethylnonadecanoate; ethyl 3-hydroxy-10,17-dimethylnonadecanoate; ethyl 10,15-dimethylhexadecanoate; ethyl 10,15-dimethylheptadecanoate; propyl 10,17-dimethyloctadecanoate; propyl 3-hydroxy-10,17-dimethyloctadecanoate; propyl 10,17-dimethylnonadecanoate; propyl 3-hydroxy-10,17-dimethylnonadecanoate; propyl 10,15-dimethylhexadecanoate; propyl 10,15-dimethylheptadecanoate; or mixture(s) thereof.

Fatty acid esters of the present disclosure can be produced using any suitable reaction conditions for esterification of carboxylic acids or transesterification of esters. For example, an alkylated fatty acid of the present disclosure can be introduced to an alcohol (e.g., methanol, ethanol, and/or propanol, etc.) followed by addition of a sodium alkoxide and incubating the mixture at any suitable temperature (e.g., about 23° C.). In at least one embodiment, a sodium alkoxide is sodium methoxide, sodium ethoxide, sodium propoxide, or mixture(s) thereof. The mixture can be incubated for any suitable time, such as from about 10 minutes to about 10 hours, such as from about 1 hour to about 5 hours, such as from about 2.5 hours to about 3.5 hours. The reaction can be quenched with an acid solution (e.g., 2 N hydrochloric acid), and the fatty acid esters can be extracted using any suitable non-polar solvent (such as hexane(s)). The fatty acid ester products can be dried under vacuum or inert gas (e.g., nitrogen).

In at least one embodiment, the kinematic viscosity at 100° C. of a fatty acid ester can be less than about 10 cSt, such as less than about 6 cSt, such as less than about 4.5 cSt, such as less than about 3.2 cSt, such as from about 2.8 cSt to about 4.5 cSt.

In at least one embodiment, the kinematic viscosity at 40° C. of a fatty acid ester can be less than about 25 cSt, such as less than about 15 cSt.

In at least one embodiment, the pour point of a fatty acid ester can be below about −30° C., such as below about −40° C., such as below about −50° C., such as below about −60° C., such as below about −70° C., such as below about −80° C.

In at least one embodiment, the Noack volatility of a fatty acid ester can be less than about 19 wt %, such as less than about 14 wt %, such as less than about 12 wt %, such as less than about 10 wt %, such as less than about 9.0 wt %, such as less than about 8.5 wt %, such as less than about 8.0 wt %, such as less than about 7.5 wt %.

In at least one embodiment, the viscosity index of a fatty acid ester can be more than about 120, such as more than about 121, such as more than about 125, such as more than about 130, such as more than about 135, such as more than about 136.

In at least one embodiment, the cold crank simulator value (CCS) at −35° C. of a fatty acid ester may be not more than about 1200 cP, such as not more than about 1000 cP, such as not more than about 900 cP.

In at least one embodiment, a fatty acid ester can have a Brookfield viscosity at 40° C. of less than about 3000 cP, such as less than about 2000 cP, such as less than about 1500 cP.

In at least one embodiment, a fatty acid ester can have a rotating pressure vessel oxidation test (RPVOT) of about 70 min or more, such as about 80 min or more.

In at least one embodiment, a fatty acid ester can have a kinematic viscosity at 100° C. of not more than about 3.2 cSt and a Noack volatility of not more than about 19 wt %. In at least one embodiment, the alkane product can have a kinematic viscosity at 100° C. of not more than about 3.6 cSt and a Noack volatility of not more than about 12.5 wt %.

### Alkane Product Formation

In some embodiments, a fatty acid (such as an alkylated fatty acid) can be formed into an alkane product using one or more biological pathways. For example, a fatty acid (such as an alkylated fatty acid) can be introduced to a reductase (such as an AAR protein (SEQ ID NO. 162)) and NADPH to form an aldehyde intermediate fatty acid (such as an aldehyde intermediate alkylated fatty acid). The aldehyde intermediate can be treated with an (1) oxygen source and (2) a decarbonylase. In at least one embodiment, a decarbonylase is selected from CYP4G protein (SEQ ID NO. 163), ADO protein (SEQ ID NO. 164), CER1 protein (SEQ ID NO. 165), or combination(s) thereof. The alkane product may be formed in one or more bioreactors, similar to as described above for reductase, methyl transferase, and terminal fatty acid alkylation.

A reductase gene (e.g., an unmodified reductase gene or recombinant reductase gene) may comprise a naturally-occurring nucleotide sequence set forth in SEQ ID NO:166.

A reductase gene may be derived from any host cell suitable for expression of a reductase gene, such as fungal, bacterial, plant, animal, or yeast species, such as Synechococcus elongates.

A decarbonylase gene (e.g., an unmodified decarbonylase gene or recombinant decarbonylase gene) may comprise a naturally-occurring nucleotide sequence set forth in SEQ ID NO:167.

A decarbonylase gene may be derived from any host cell suitable for expression of a decarbonylase gene, such as fungal, bacterial, plant, animal, or yeast species, such as Arabidopsis thaliana or Drosophila melanogaster.

A reductase and/or decarbonylase gene may be native or the reductase and/or decarbonylase gene may be recombinant because it is operably-linked to a promoter other than the naturally-occurring promoter of the reductase gene or decarbonylase gene, respectively. Such genes may be useful to drive transcription in a particular species of cell. A recombinant reductase and/or decarbonylase gene may be recombinant because it contains one or more nucleotide substitutions relative to a naturally-occurring reductase gene or decarbonylase gene, respectively. Such genes may be useful to increase the translation efficiency of the reductase gene's mRNA transcript or decarbonylase's mRNA transcript in a particular species of cell.

A nucleic acid may comprise a reductase gene and/or a decarbonylase gene and a promoter, wherein the gene and promoter are operably-linked. The gene and promoter may be derived from different species. A cell may comprise the nucleic acid, and the promoter may be capable of driving transcription in the cell. A cell may comprise a reductase gene and/or a decarbonylase gene, and may be operably-linked to a promoter capable of driving transcription of the gene in the cell. The cell may be a species of yeast, and the promoter may be a yeast promoter. The cell may be a species of bacteria, and the promoter may be a bacterial promoter. The cell may be a species of algae, and the promoter may be an algae promoter. The cell may be a species of plant, and the promoter may be a plant promoter.

A reductase gene and/or a decarbonylase gene may be operably-linked to a promoter that cannot drive transcription in the cell from which the gene originated. For example, the promoter may not be capable of binding an RNA polymerase of the cell from which a reductase gene and/or a decarbonylase gene originated. In some embodiments, the promoter cannot bind a prokaryotic RNA polymerase and/or initiate transcription mediated by a prokaryotic RNA polymerase. In some embodiments, a reductase gene and/or a decarbonylase gene is operably-linked to a promoter that cannot drive transcription in the cell from which the protein encoded by the gene originated. For example, the promoter may not be capable of binding an RNA polymerase of a cell that naturally expresses a reductase and/or a decarbonylase enzyme encoded by a reductase gene or a decarbonylase gene, respectively.

A promoter may be an inducible promoter or a constitutive promoter. A promoter may be any one of the promoters described above.

A reductase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in the naturally-occurring nucleotide sequences set forth in SEQ ID NO:166. A reductase gene may comprise a nucleotide sequence with, with at least, with at most 65%, 70%, 75%, 80%, 85%, 90%, 95%, 96%, 97%, 98%, or 99% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:166. A reductase gene may or may not have 100% sequence identity with any one of the nucleotide sequences set forth in nucleotide sequences set forth in SEQ ID NO: 166. A reductase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO: 166. A reductase gene may comprise a nucleotide sequence with, with at least, or with at most 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, 99%, or 100% sequence identity with the nucleotide sequence set forth in SEQ ID NO: 166, and the reductase gene may encode a reductase protein with at least about 95%, 96%, 97%, 98%, 99%, or 100% sequence identity with the amino acid sequence set forth in SEQ ID NO:162. For example, the protein encoded by SEQ ID NO:166 does not have 100% sequence identity with the amino acid sequence set forth in SEQ ID NO:162.

In some embodiments, the reductase gene encodes a reductase protein that has specific amino acids unchanged from the amino acid sequence set forth in SEQ ID NO:162.

A recombinant reductase gene may vary from a naturally-occurring reductase gene because the recombinant reductase gene may be codon-optimized for expression in a eukaryotic cell, such as a plant cell, algae cell, or yeast cell. A cell may comprise a recombinant reductase gene, wherein the recombinant reductase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant reductase gene may vary from a naturally-occurring reductase gene or may be unchanged from a naturally-occurring reductase gene. For example, a recombinant reductase gene may comprise a nucleotide sequence with at least 65% sequence identity with the naturally-occurring nucleotide sequence set forth in SEQ ID NO:166, (e.g., at least 70%, 75%, 80%, 85%, 90%, 95%, 96%, 97%, 98%, or 99% sequence identity), and at least 5 codons of the nucleotide sequence of the recombinant reductase gene may vary from the naturally-occurring nucleotide sequence (e.g., at least 10, 15, 20, 25, 30, 35, 40, 45, 50, 60, 70, 80, 90, or 100 codons).

The sequences of naturally-occurring reductase proteins are set forth in SEQ ID NO:162. A recombinant reductase gene may or may not encode a protein comprising 100% sequence identity with the amino acid sequence set forth in SEQ ID NO:162. For example, a recombinant reductase gene may encode a protein having 100% sequence identity with a biologically-active portion of an amino acid sequence set forth in SEQ ID NO:162.

Substrates for the reductase protein may include any fatty acid from 12 to 20 carbons long with a methyl or methylene substitution in the 7, 8, 9, 10, 11, or 12 position. The fatty acid substrate may be 12, 13, 14, 15, 16, 17, 18, 19, or 20 carbons long, or any range derivable therein. Additionally or alternatively, substrates for the reductase protein can be a reaction product of the methylase, internal reductase, and/or terminal alkyl transferase described above. For example, a substrate can be a methylated fatty acid from 12 to 20 carbons long with a methyl substitution in the 7, 8, 9, 10, 11, or 12 position and a methyl or ethyl substitution at a terminal carbon.

The fatty acid that has been treated with a reductase can form an aldehyde-containing fatty acid derivative (e.g., an aldehyde is present in the fatty acid molecule where a carboxylic acid moiety was present before treatment with the reductase). The aldehyde-containing fatty acid derivative can be treated with (1) an oxygen source and (2) a decarbonylase in the same bioreactor or a different bioreactor as that used to form the aldehyde-containing fatty acid derivative. The oxygen source can be any suitable oxygen source, such as 02.

A decarbonylase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in the naturally-occurring nucleotide sequences set forth in SEQ ID NO:167. A decarbonylase gene may comprise a nucleotide sequence with, with at least, with at most 65%, 70%, 75%, 80%, 85%, 90%, 95%, 966, 976, 986, or 996 sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:167. A decarbonylase gene may or may not have 100% sequence identity with any one of the nucleotide sequences set forth in nucleotide sequences set forth in SEQ ID NO:167. A decarbonylase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO:167. A decarbonylase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, 99%, or 100% sequence identity with the nucleotide sequence set forth in SEQ ID NO: 167, and the decarbonylase gene may encode a decarbonylase protein with at least about 95%, 96%, 97%, 98%, 99%, or 100% sequence identity with the amino acid sequence set forth in SEQ ID NO:163, SEQ ID NO:164, or SEQ ID NO:165. For example, the protein encoded by SEQ ID NO:167 does not have 100% sequence identity with the amino acid sequence set forth in SEQ ID NO:165.

In some embodiments, the decarbonylase gene encodes a decarbonylase protein that has specific amino acids unchanged from the amino acid sequence set forth in SEQ ID NO:163, SEQ ID NO:164, or SEQ ID NO:165.

A recombinant decarbonylase gene may vary from a naturally-occurring decarbonylase gene because the recombinant decarbonylase gene may be codon-optimized for expression in a eukaryotic cell, such as a plant cell, algae cell, or yeast cell. A cell may comprise a recombinant decarbonylase gene, wherein the recombinant decarbonylase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant decarbonylase gene may vary from a naturally-occurring decarbonylase gene or may be unchanged from a naturally-occurring decarbonylase gene. For example, a recombinant decarbonylase gene may comprise a nucleotide sequence with at least 65% sequence identity with the naturally-occurring nucleotide sequence set forth in SEQ ID NO:167, (e.g., at least 70%, 75%, 80%, 85%, 90%, 95%, 96%, 97%, 98%, or 99% sequence identity), and at least 5 codons of the nucleotide sequence of the recombinant decarbonylase gene may vary from the naturally-occurring nucleotide sequence (e.g., at least 10, 15, 20, 25, 30, 35, 40, 45, 50, 60, 70, 80, 90, or 100 codons).

The sequences of naturally-occurring decarbonylase proteins are set forth in SEQ ID NO:163, SEQ ID NO:164, and SEQ ID NO:165. A recombinant decarbonylase gene may or may not encode a protein comprising 100% sequence identity with the amino acid sequence set forth in SEQ ID NO:163, SEQ ID NO:164, or SEQ ID NO:165. For example, a recombinant decarbonylase gene may encode a protein having 100% sequence identity with a biologically-active portion of an amino acid sequence set forth in SEQ ID NO:163, SEQ ID NO:164, or SEQ ID NO:165.

Fatty acid products of the decarbonylase process are alkanes (alkane products, e.g., decarbonylated fatty acid derivatives).

An alkane can have:

a methyl substituent;

(1) an ethyl substituent or (2) an additional methyl substituent, wherein the ethyl substituent or the additional methyl substituent is located at a carbon atom alpha to the terminal carbon atom of a fatty acid; and/or

optionally an alcohol substituent.

Alkane products of the present disclosure can include 6-methylundecane; 2,6-dimethylundecane; 3,7-dimethyldodecane; 6-methyltridecane; 2,6-dimethyltridecane; 3,7-dimethyltetradecane; 2,9-dimethylheptadecane; 2,7-dimethylpentadecane; 3,8-dimethylhexadecane; or mixture(s) thereof.

### Alkenylated (Alkene) Product Formation

In some embodiments, a fatty acid (such as an alkylated fatty acid) can be formed into an alkenylated (alkene) product using one or more biological pathways. For example, a fatty acid (such as an alkylated fatty acid) can be introduced to a decarboxylase (e.g., a decarboxylase that is an alpha olefinase, e.g. a decarboxylase configured to form alkylated fatty acid alpha olefins). A decarboxylase can be a P450 fatty acid decarboxylase from Macrococcus caseolyticus, Jeotgalicoccus, or Synechococcus (e.g., sp. Strain PCC 7002) Species. In at least one embodiment, a decarboxylase is selected from OleTje protein (SEQ ID NO. 168), OleTmc protein (SEQ ID NO. 169), Ols protein (SEQ ID NO. 170), UndA protein (SEQ ID NO. 171), UndB protein (SEQ ID NO. 172), and combination(s) thereof. The alkenylated (alkene) product may be formed in one or more bioreactors, similar to as described above for reductase, methyl transferase, and terminal alkylation.

A decarboxylase gene (e.g., an unmodified decarboxylase gene or recombinant decarboxylase gene) may comprise a naturally-occurring nucleotide sequence set forth in SEQ ID NO:173 (encodes OleTje protein), SEQ ID NO:174 (encodes OleTmc protein), SEQ ID NO:175 (encodes Ols protein), SEQ ID NO:176 (encodes UndB protein and UndA protein).

A decarboxylase gene may be derived from any host cell suitable for expression of a decarboxylase gene, such as fungal, bacterial, plant, animal, or yeast species, such as Alicycloba cillus acidocaldarius, Staphylococcus massiliensis, Saccharomyces cerevisiae, Macrococcus caseolyticus, Pseudomonas protegens, or Jeotgalicoccus Species.

A decarboxylase gene may be native or the decarboxylase gene may be recombinant because it is operably-linked to a promoter other than the naturally-occurring promoter of the decarboxylase gene. Such genes may be useful to drive transcription in a particular species of cell. A recombinant decarboxylase gene may be recombinant because it contains one or more nucleotide substitutions relative to a naturally-occurring decarboxylase gene. Such genes may be useful to increase the translation efficiency of the reductase gene's mRNA transcript or decarboxylase's mRNA transcript in a particular species of cell.

A nucleic acid may comprise a decarboxylase gene and a promoter, wherein the gene and promoter are operably-linked. The gene and promoter may be derived from different species. A cell may comprise the nucleic acid, and the promoter may be capable of driving transcription in the cell. A cell may comprise a decarboxylase gene, and may be operably-linked to a promoter capable of driving transcription of the gene in the cell. The cell may be a species of yeast, and the promoter may be a yeast promoter. The cell may be a species of bacteria, and the promoter may be a bacterial promoter. The cell may be a species of algae, and the promoter may be an algae promoter. The cell may be a species of plant, and the promoter may be a plant promoter.

A decarboxylase gene may be operably-linked to a promoter that cannot drive transcription in the cell from which the gene originated. For example, the promoter may not be capable of binding an RNA polymerase of the cell from which a decarboxylase gene originated. In some embodiments, the promoter cannot bind a prokaryotic RNA polymerase and/or initiate transcription mediated by a prokaryotic RNA polymerase. In some embodiments, a decarboxylase gene is operably-linked to a promoter that cannot drive transcription in the cell from which the protein encoded by the gene originated. For example, the promoter may not be capable of binding an RNA polymerase of a cell that naturally expresses a decarboxylase enzyme encoded by a decarboxylase gene.

A promoter may be an inducible promoter or a constitutive promoter. A promoter may be any one of the promoters described above.

A decarboxylase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence set forth in the naturally-occurring nucleotide sequences set forth in SEQ ID NO:173, SEQ ID NO:174, SEQ ID NO:175, or SEQ ID NO:176. A decarboxylase gene may comprise a nucleotide sequence with, with at least, with at most 65%, 70%, 75%, 80%, 85%, 90%, 95%, 96%, 97%, 98%, or 99% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs starting at nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, or 1200 of the nucleotide sequence set forth in SEQ ID NO:173, SEQ ID NO:174, SEQ ID NO:175, or SEQ ID NO:176. A decarboxylase gene may or may not have 100% sequence identity with any one of the nucleotide sequences set forth in nucleotide sequences set forth in SEQ ID NO:173, SEQ ID NO:174, SEQ ID NO:175, or SEQ ID NO:176. A decarboxylase gene may or may not have 100% sequence identity with 150, 200, 250, 300, 350, 400, 450, 500, 550, 600, 650, 700, 750, 800, 850, 900, 950, 1000, 1050, 1100, 1150, 1200, 1250, or 1300 contiguous base pairs of the nucleotide sequence set forth in SEQ ID NO:173, SEQ ID NO:174, SEQ ID NO:175, or SEQ ID NO:176. A decarboxylase gene may comprise a nucleotide sequence with, with at least, or with at most 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, 99%, or 100% sequence identity with the nucleotide sequence set forth in SEQ ID NO:173, SEQ ID NO:174, SEQ ID NO:175, or SEQ ID NO:176, and the decarboxylase gene may encode a decarboxylase protein with at least about 95%, 96%, 97%, 98%, 99%, or 100% sequence identity with the amino acid sequence set forth in SEQ ID NO:168, SEQ ID NO:169, SEQ ID NO:170, SEQ ID NO:171, or SEQ ID NO:172. For example, the protein encoded by SEQ ID NO:173 does not have 100% sequence identity with the amino acid sequence set forth in SEQ ID NO:168.

In some embodiments, the decarboxylase gene encodes a decarboxylase protein that has specific amino acids unchanged from the amino acid sequence set forth in SEQ ID NO:168, SEQ ID NO:169, SEQ ID NO:170, SEQ ID NO:171, or SEQ ID NO:172.

A recombinant decarboxylase gene may vary from a naturally-occurring decarboxylase gene because the recombinant decarboxylase gene may be codon-optimized for expression in a eukaryotic cell, such as a plant cell, algae cell, or yeast cell. A cell may comprise a recombinant decarboxylase gene, wherein the recombinant decarboxylase gene is codon-optimized for the cell.

Exactly, at least, or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 codons of a recombinant decarboxylase gene may vary from a naturally-occurring decarboxylase gene or may be unchanged from a naturally-occurring decarboxylase gene. For example, a recombinant decarboxylase gene may comprise a nucleotide sequence with at least 65% sequence identity with the naturally-occurring nucleotide sequence set forth in SEQ ID NO:173, SEQ ID NO:174, SEQ ID NO:175, or SEQ ID NO:176, (e.g., at least 70%, 75%, 80%, 85%, 90%, 95%, 96%, 97%, 98%, or 99% sequence identity), and at least 5 codons of the nucleotide sequence of the recombinant decarboxylase gene may vary from the naturally-occurring nucleotide sequence (e.g., at least 10, 15, 20, 25, 30, 35, 40, 45, 50, 60, 70, 80, 90, or 100 codons).

The sequences of naturally-occurring decarboxylase proteins are set forth in SEQ ID NO:168, SEQ ID NO:169, SEQ ID NO:170, SEQ ID NO:171, and SEQ ID NO:172. A recombinant decarboxylase gene may or may not encode a protein comprising 100% sequence identity with the amino acid sequence set forth in SEQ ID NO:168, SEQ ID NO:169, SEQ ID NO:170, SEQ ID NO:171, or SEQ ID NO:172. For example, a recombinant decarboxylase gene may encode a protein having 100% sequence identity with a biologically-active portion of an amino acid sequence set forth in SEQ ID NO:168, SEQ ID NO:169, SEQ ID NO:170, SEQ ID NO:171, or SEQ ID NO:172.

Substrates for the decarboxylase protein may include any fatty acid from 12 to 20 carbons long with a methyl or methylene substitution in the 7, 8, 9, 10, 11, or 12 position. The fatty acid substrate may be 12, 13, 14, 15, 16, 17, 18, 19, or 20 carbons long, or any range derivable therein. Additionally or alternatively, substrates for the decarboxylase protein can be a reaction product of the methylase, internal reductase, and/or terminal alkyl transferase described above. For example, a substrate can be a methylated fatty acid from 12 to 20 carbons long with a methyl substitution in the 7, 8, 9, 10, 11, or 12 position and a methyl or ethyl substitution at a terminal carbon.

The fatty acid that has been treated with a decarboxylase can form an alkene-containing fatty acid derivative (alkenylated products, e.g., an alkene is present in the fatty acid molecule where a carboxylic acid moiety was present before treatment with the decarboxylase).

Alkene products can have:
an olefin moiety;
a methyl substituent;
(1) an ethyl substituent or (2) an additional methyl substituent, wherein
the ethyl substituent or the additional methyl substituent is located at a carbon atom alpha to the terminal carbon atom of the fatty acid; and
optionally an alcohol substituent.

The alkene can have:
the methyl substituent located at a 11 carbon atom,
(1) the ethyl substituent or (2) the additional methyl substituent located at a carbon atom selected from the group consisting of 15, 16, 17, or 18, and/or
the olefin located at a 1 carbon atom.

Alkene products of the present disclosure can include 6-methylundec-1-ene; 6,10-dimethylundec-1-ene; 6,10-dimethyldodec-1-ene; 8-methyltridec-1-ene; 8,12-dimethyltridec-1-ene; 8,12-dimethyltetradec-1-ene; 9,16-dimethylheptadec-1-ene; 9,14-dimethylpentadec-1-ene; 9,14-dimethylhexadec-1-ene; 9,16-dimethylheptadec-1-ene; or mixture(s) thereof.

### EMBODIMENTS LISTING

The present disclosure provides, among others, the following embodiments, each of which may be considered as optionally including any alternate embodiments.

Clause 1. A process comprising:

introducing a terminal alkyl transferase and a fatty acid into a bioreactor;

introducing an internal methyl transferase and optionally internal methyl reductase into the bioreactor or a second bioreactor; and

obtaining an alkylated fatty acid having a methyl substituent located at an internal carbon atom of the fatty acid and a terminal methyl substituent or terminal ethyl substituent located at a carbon atom alpha to the terminal carbon atom of the fatty acid.

Clause 2. The process of Clause 1, wherein the alkylated fatty acid has a terminal methyl or ethyl substituent.

Clause 3. The process of Clauses 1 or 2, wherein the terminal alkyl transferase is a □-ketoacyl-acyl carrier protein synthase.

Clause 4. The process of any of Clauses 1 to 3, wherein the □-ketoacyl-acyl carrier protein synthase has 95% or greater sequence identity to an amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161.

Clause 5. The process of any of Clauses 1 to 4, wherein the alkylated fatty acid has a terminal ethyl substituent.

Clause 6. The process of any of Clauses 1 to 5, wherein introducing the terminal alkyl transferase into the bioreactor comprises introducing an alkyl transferase gene to the bioreactor, wherein the alkyl transferase gene expresses the terminal alkyl transferase.

Clause 7. The process of any of Clauses 1 to 6, wherein the alkyl transferase gene is configured to encode the terminal alkyl transferase protein of a gram-negative species of Proteobacterium or gram-positive species of Firmicute.

Clause 8. The process of any of Clauses 1 to 7, wherein alkyl transferase gene is selected from the group consisting of: (1) a FabH gene having greater than 95% sequence identity to the nucleic acid sequence of SEQ ID NO. 142, (2) an eFabH gene having greater than 95% sequence identity to the nucleic acid sequence of SEQ ID NO. 146, (3) a bFabH1 gene having greater than 95% sequence identity to the nucleic acid sequence of SEQ ID NO. 140, (4) a bFabH2 gene having greater than 95% sequence identity to the nucleic acid sequence of SEQ ID NO. 144, and (6) combination(s) thereof.

Clause 9. The process of any of Clauses 1 to 8, wherein introducing the terminal alkyl transferase into the bioreactor comprises introducing a cell suitable for expression of a terminal alkyl transferase gene, the cell selected from the group consisting of Bacillus, Haemophilus, Vibrio harvevi, Rhodobacter, Escherichia, Staphylococci, Streptomycete, and combination(s) thereof.

Clause 10. The process of any of Clauses 1 to 9, wherein introducing the methyl transferase and optionally the internal methyl reductase into the bioreactor comprises introducing a cell configured to express the methyl transferase gene and optionally the internal methyl reductase gene, the cell selected from the group consisting of Mycobacteria, Corynebacteria, Nocardia, Streptomyces, Rhodococcus, and combination(s) thereof.

Clause 11. The process of any of Clauses 1 to 10, wherein the methyl transferase has 95% or greater sequence identity to an amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125.

Clause 12. The process of any of Clauses 1 to 11, wherein the internal methyl reductase has 95% or greater sequence identity to an amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO 115, SEQ ID NO: 119, or SEQ ID NO 123.

Clause 13. The process of any of Clauses 1 to 12, wherein the fatty acid is selected from the group consisting of oleic acid, myristoleic acid, palmitoleic acid, and combination(s) thereof.

Clause 14. The process of any of Clauses 1 to 13, further comprising introducing, into the bioreactor, methionine, s-adenosyl methionine, a Coenzyme-A, an acyl carrier protein, Q-mercaptoethanol, NADPH, NADH, urea, glycerol, methionine, thiamine, Q-alanine, ampicillin, or combination(s) thereof.

Clause 15. The process of any of Clauses 1 to 14, wherein obtaining the alkylated fatty acid comprises extracting the alkylated fatty acid from the bioreactor using an organic solvent.

Clause 16. The process of any of Clauses 1 to 15, wherein obtaining the alkylated fatty acid comprises introducing a bioreactor effluent to a settling tank and decanting the alkylated fatty acid from the settling tank.

Clause 17. The process of any of Clauses 1 to 16, further comprising:

removing a first effluent from the bioreactor;

introducing the first effluent to a settling tank;

removing a second effluent from the settling tank;

introducing the second effluent to the second bioreactor; and

removing a third effluent from the second bioreactor,

wherein obtaining the alkylated fatty acid comprises:

introducing the third effluent to a settling tank; and

removing a fourth effluent from the settling tank, the fourth effluent comprising the alkylated fatty acid.

Clause 18. The process of any of Clauses 1 to 17, further comprising:

removing a first effluent from the second bioreactor;

introducing the first effluent to a settling tank;

removing a second effluent from the settling tank;

introducing the second effluent to the first bioreactor; and

removing a third effluent from the first bioreactor,

wherein obtaining the alkylated fatty acid comprises:

introducing the third effluent to a settling tank; and

removing a fourth effluent from the settling tank, the fourth effluent comprising the alkylated fatty acid.

Clause 19. The process of any of Clauses 1 to 18, wherein the process comprises introducing the internal methyl transferase into the bioreactor, the process further comprising:

removing a first effluent from the bioreactor,

wherein obtaining the alkylated fatty acid comprises:

introducing the first effluent to a settling tank; and

removing a second effluent from the settling tank, the second effluent comprising the alkylated fatty acid.

Clause 20. The process of any of Clauses 1 to 19, wherein the alkylated fatty acid comprises a methyl branch at the 7, 8, 9, 10, 11, or 12 position.

Clause 21. The process of any of Clauses 1 to 20, further comprising introducing into the bioreactor a TmsC protein having 95% or greater sequence identity to an amino acid sequence set forth in SEQ ID NO:66, SEQ ID NO:68, SEQ ID NO:70, SEQ ID NO:72, SEQ ID NO:74, SEQ ID NO:76, SEQ ID NO:129, SEQ ID NO:131, SEQ ID NO:133, or SEQ ID NO:135.

Clause 22. The process of any of Clauses 1 to 21, wherein the alkylated fatty acid is selected from the group consisting of 7,11-dimethyldodecanoic acid; 7,11-dimethyltridecanoic acid; 9,13-dimethyltetradecanoic acid; 9,13-dimethylpentadecanoic acid; 10,17-dimethylstearic acid; 3-hydroxy-10,17-dimethyloctadecanoic acid; 10,17-dimethylnonadecanoic acid; 3-hydroxy-10,17-dimethylnonadecanoic acid; 10,15-dimethylhexadecanoic acid; 10,15-dimethylheptadecanoic acid; and combination(s) thereof.

Clause 23. The process of any of Clauses 1 to 22, wherein the alkylated fatty acid comprises a 10-methyl,17-methyl fatty acid.

Clause 24. A fatty acid ester having:

a methyl substituent;

(1) an ethyl substituent or (2) an additional methyl substituent, wherein the ethyl substituent or the additional methyl substituent is located at a carbon atom alpha to the terminal carbon atom of the fatty acid; and

optionally an alcohol substituent.

Clause 25. The fatty acid ester of Clause 24, wherein:

the methyl substituent is located at carbon number 11, and

(1) the ethyl substituent or (2) the additional methyl substituent is located at a carbon atom selected from the group consisting of carbon number 13, 14, 15, 16, 17, 18, and 19.

Clause 26. The fatty acid ester of Clauses 24 or 25, wherein the fatty acid ester is selected from the group consisting of methyl 7,11-dimethyldodecanoate; methyl 7,11-dimethyltridecanoate; methyl9,13-dimethyltetradecanoate; methyl9,13-dimethylpentadecanoate; methyl 10,17-dimethyloctadecanoate; methyl 3-hydroxy-10,17-dimethyloctadecanoate; methyl 10,17-dimethylnonadecanoate; methyl 3-hydroxy-10,17-dimethylnonadecanoate; methyl 10,15-dimethylhexadecanoate; methyl 10,15-dimethylheptadecanoate; ethyl 10,17-dimethyloctadecanoate; ethyl 3-hydroxy-10,17-dimethyloctadecanoate; ethyl 10,17-dimethylnonadecanoate; ethyl 3-hydroxy-10,17-dimethylnonadecanoate; ethyl 10,15-dimethylhexadecanoate; ethyl 10,15-dimethylheptadecanoate; propyl 10,17-dimethyloctadecanoate; propyl 3-hydroxy-10,17-dimethyloctadecanoate; propyl 10,17-dimethylnonadecanoate; propyl 3-hydroxy-10,17-dimethylnonadecanoate; propyl 10,15-dimethylhexadecanoate; propyl 10,15-dimethylheptadecanoate; and combination(s) thereof.

Clause 27. The fatty acid ester of any of Clauses 24 to 26, wherein the fatty acid ester has one or more of the following properties:

a kinematic viscosity at 100° C. of less than 4.5 cSt;

a kinematic viscosity at 40° C. of less than 15 cSt;

a pour point of below −50° C.;

a Noack volatility of less than 14 wt %; and

a viscosity index of more than 120.

Clause 28. A lubricant comprising the fatty acid ester of any of Clauses 24 to 27.

Clause 29. A fatty acid derivative having:

an olefin moiety;

a methyl substituent;

(1) an ethyl substituent or (2) an additional methyl substituent, wherein the ethyl substituent or the additional methyl substituent is located at a carbon atom alpha to the terminal carbon atom of the fatty acid; and

optionally an alcohol substituent.

Clause 30. The fatty acid derivative of Clause 29, wherein:

the methyl substituent is located at carbon number 11,

(1) the ethyl substituent or (2) the additional methyl substituent is located at a carbon atom selected from the group consisting of carbon number 13, 14, 15, 16, 17, 18, and 19, and

the olefin is located at a 1 carbon atom.

Clause 31. The fatty acid derivative of Clauses 29 or 30, wherein the fatty acid is selected from the group consisting of 6-methylundec-1-ene; 6,10-dimethylundec-1-ene; 6,10-dimethyldodec-1-ene; 8-methyltridec-1-ene; 8,12-dimethyltridec-1-ene; 8,12-dimethyltetradec-1-ene; 9,16-dimethylheptadec-1-ene; 9,14-dimethylpentadec-1-ene; 9,14-dimethylhexadec-1-ene;

9,16-dimethylheptadec-1-ene; and combination(s) thereof.

Clause 32. A lubricant comprising the fatty acid of any of Clauses 29 to 31.

Clause 33. A fatty acid derivative having:

a methyl substituent;

(1) an ethyl substituent or (2) an additional methyl substituent, wherein the ethyl substituent or the additional methyl substituent is located at a carbon atom alpha to the terminal carbon atom of the fatty acid; and

optionally an alcohol substituent.

Clause 34. The fatty acid derivative of Clause 33, wherein:

the methyl substituent is located at carbon number 11.

Clause 35. The fatty acid derivative of Clauses 33 or 34, wherein the fatty acid is selected from the group consisting of 6-methylundecane; 2,6-dimethylundecane; 3,7-dimethyldodecane; 6-methyltridecane; 2,6-dimethyltridecane; 3,7-dimethyltetradecane; 2,9-dimethylheptadecane; 2,7-dimethylpentadecane; 3,8-dimethylhexadecane; and combination(s) thereof.

Clause 36. A lubricant comprising the fatty acid derivative of any of Clauses 33 to 35.

Overall, processes of the present disclosure can provide longer chain, multiply-alkylated alkanes. It has been discovered that methylation toward the middle of a fatty acid molecule (in addition to alkylation at a terminus of the fatty acid) is advantageous for cetane value and cold flow properties (likely because it is breaking up the waxy structure). For example, an alkane product of the present disclosure can have a high cetane value.

Furthermore, processes of the present disclosure can be beneficial because biological addition of methyl side chains eliminates the need to catalytically isomerize linear alkanes to obtain a branched structure, thus improving yield and removing the carbon-intensive and energy-intensive catalytic reforming process in the production of biodiesels and other basestocks. The one or more methyl branches also provide useful physical properties to the alkanes.

Branched-chain fatty acids can have other varying properties when compared to straight-chain fatty acids of the same molecular weight (i.e., isomers), such as considerably lower melting points which can in turn provide lower pour points when made into industrial chemicals. These additional benefits allow the branched-chain fatty acids to confer substantially lower volatility and vapor pressure and improved stability against oxidation and rancidity. These properties make branched-chain fatty acids particularly suited as components for industrial lubricants or fuel additives.

Alkane products and ester products of the present disclosure can be formed at high yield.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the present disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

What is claimed is:

1. A process comprising:
introducing a terminal alkyl transferase and a fatty acid into a bioreactor;
introducing an internal methyl transferase and optionally an internal methyl reductase into the bioreactor or a second bioreactor; and
obtaining an alkylated fatty acid having a methyl substituent located at an internal carbon atom of the fatty acid and a terminal methyl substituent or terminal ethyl substituent located at a carbon atom alpha to the terminal carbon atom of the fatty acid.

2. The process of claim 1, wherein the alkylated fatty acid has a terminal methyl substituent.

3. The process of claim 1, wherein the terminal alkyl transferase is a β-ketoacyl-acyl carrier protein synthase.

4. The process of claim 2, wherein the β-ketoacyl-acyl carrier protein synthase has 95% or greater sequence identity to an amino acid sequence set forth in SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, or SEQ ID NO:161.

5. The process of claim 1, wherein the alkylated fatty acid has a terminal ethyl substituent.

6. The process of claim 1, wherein introducing the terminal alkyl transferase into the bioreactor comprises introducing an alkyl transferase gene to the bioreactor, wherein the alkyl transferase gene expresses the terminal alkyl transferase.

7. The process of claim 6, wherein the alkyl transferase gene is configured to encode the terminal alkyl transferase protein of a Proteobacterium or species of Firmicute.

8. The process of claim 6, wherein alkyl transferase gene is selected from the group consisting of: (1) a FabH gene having greater than 95% sequence identity to the nucleic acid sequence of SEQ ID NO. 142, (2) an eFabH gene having greater than 95% sequence identity to the nucleic acid sequence of SEQ ID NO. 146, (3) a bFabH1 gene having greater than 95% sequence identity to the nucleic acid sequence of SEQ ID NO. 140, (4) a bFabH2 gene having greater than 95% sequence identity to the nucleic acid sequence of SEQ ID NO. 144, and (6) combination(s) thereof.

9. The process of claim 1, wherein introducing the terminal alkyl transferase into the bioreactor comprises introducing a cell suitable for expression of a terminal alkyl transferase gene, the cell selected from the group consisting of Bacillus, Haemophilus, Vibrio harvevi, Rhodobacter, Escherichia, Staphylococci, Streptomycete, and combination(s) thereof.

10. The process of claim 1, wherein introducing the methyl transferase into the bioreactor comprises introducing a cell configured to express the methyl transferase gene, the cell selected from the group consisting of Bacillus, Haemophilus, Vibrio harvevi, Rhodobacter, Escherichia, Staphylococci, Streptomycete, Saccharomyces cerevisiae, Pichia Pastoris, Corynebacteria, and combination(s) thereof.

11. The process of claim 10, further comprising introducing the internal methyl reductase into the bioreactor by introducing a cell configured to express the internal methyl reductase gene, the cell selected from the group consisting of Bacillus, Haemophilus, Vibrio harvevi, Rhodobacter, Escherichia, Staphylococci, Streptomycete, Escherichia, Saccharomyces, Pichia, Corynebacteria and combination(s) thereof.

12. The process of claim 10, wherein the methyl transferase has 95% or greater sequence identity to an amino acid sequence set forth in SEQ ID NO:2, SEQ ID No:4, SEQ ID No:6, SEQ ID No:8, SEQ ID No:10, SEQ ID No:12, SEQ ID No:14, SEQ ID No:16, SEQ ID No:18, SEQ ID No:20, SEQ ID No:22, SEQ ID No:24, SEQ ID No:26, SEQ ID No:28, SEQ ID No:30, SEQ ID No:32, SEQ ID No: 117, or SEQ ID No:125.

13. The process of claim 11, wherein the internal methyl reductase has 95% or greater sequence identity to an amino acid sequence set forth in SEQ ID NO:34, SEQ ID NO:36, SEQ ID NO:38, SEQ ID NO:40, SEQ ID NO:42, SEQ ID NO:44, SEQ NO:46, SEQ ID NO:48, SEQ ID NO:50, SEQ NO:52, SEQ ID NO:54, SEQ ID NO:56, SEQ ID NO:58, SEQ ID NO:60, SEQ ID NO:62, SEQ ID NO:64, SEQ ID NO:101, SEQ ID NO:107, SEQ ID NO 115, SEQ ID NO: 119, or SEQ ID NO 123.

14. The process of claim 1, wherein the fatty acid is selected from the group consisting of oleic acid, myristoleic acid, palmitoleic acid, and combination(s) thereof.

15. The process of claim 1, further comprising introducing, into the bioreactor, methionine, s-adenosyl methionine, a Coenzyme-A, an acyl carrier protein, β-mercaptoethanol, NADPH, NADH, urea, glycerol, methionine, thiamine, β-alanine, ampicillin, or combination(s) thereof.

16. The process of claim 1, wherein obtaining the alkylated fatty acid comprises extracting the alkylated fatty acid from the bioreactor using an organic solvent.

17. The process of claim 1, wherein obtaining the alkylated fatty acid comprises introducing a bioreactor effluent to a centrifuge or settling tank and decanting the alkylated fatty acid from the settling tank.

18. The process of claim 1, further comprising:
removing a first effluent from the bioreactor;
introducing the first effluent to a settling tank;
removing a second effluent from the settling tank;
introducing the second effluent to the second bioreactor; and
removing a third effluent from the second bioreactor,
wherein obtaining the alkylated fatty acid comprises:
introducing the third effluent to a settling tank; and
removing a fourth effluent from the settling tank, the fourth effluent comprising the alkylated fatty acid.

19. The process of claim 1, further comprising:
removing a first effluent from the second bioreactor;
introducing the first effluent to a settling tank;
removing a second effluent from the settling tank;
introducing the second effluent to the first bioreactor; and
removing a third effluent from the first bioreactor,
wherein obtaining the alkylated fatty acid comprises:
introducing the third effluent to a settling tank; and
removing a fourth effluent from the settling tank, the fourth effluent comprising the alkylated fatty acid.

20. The process of claim 1, wherein the process comprises introducing the internal methyl transferase into the bioreactor, the process further comprising:
removing a first effluent from the bioreactor,
wherein obtaining the alkylated fatty acid comprises:
introducing the first effluent to a settling tank; and
removing a second effluent from the settling tank, the second effluent comprising the alkylated fatty acid.

21. The process of claim 1, wherein the alkylated fatty acid comprises a methyl branch at the 7, 8, 9, 10, 11, or 12 position.

22. The process of claim 1, further comprising introducing into the bioreactor a TmsC protein having 95% or greater sequence identity to an amino acid sequence set forth in SEQ ID NO:66, SEQ ID NO:68, SEQ ID NO:70, SEQ ID NO:72, SEQ ID NO:74, SEQ ID NO:76, SEQ ID NO:129, SEQ ID NO:131, SEQ ID NO:133, or SEQ ID NO:135.

23. The process of claim 1, wherein the alkylated fatty acid is selected from the group consisting of 7,11-dimethyldodecanoic acid; 7,11-dimethyltridecanoic acid; 9,13-dimethyltetradecanoic acid; 9,13-dimethylpentadecanoic acid; 10,17-dimethylstearic acid; 3-hydroxy-10,17-dimethyloctadecanoic acid; 10,17-dimethylnonadecanoic acid; 3-hydroxy-10,17-dimethylnonadecanoic acid; 10,15-dimethylhexadecanoic acid; 10,15-dimethylheptadecanoic acid; and combination(s) thereof.

24. The process of claim 1, wherein the alkylated fatty acid comprises a 10-methyl,17-methyl fatty acid.

25. A fatty acid ester having:
a methyl substituent;
(1) an ethyl substituent or (2) an additional methyl substituent, wherein the ethyl substituent or the additional methyl substituent is located at a carbon atom alpha to the terminal carbon atomof the fatty acid; and
optionally an alcohol substituent.

26. The fatty acid ester of claim 25, wherein:
the methyl substituent is located at carbon number 11, and
(1) the ethyl substituent or (2) the additional methyl substituent is located at a carbon atom selected from the group consisting of carbon number 13, 14, 15, 16, 17, 18, and 19.

27. The fatty acid ester of claim 25, wherein the fatty acid ester is selected from the group consisting of methyl 7,11-dimethyldodecanoate; methyl 9,13-dimethyltetradecanoate; methyl 9,13-dimethylpentadecanoate; methyl 7,11-dimethyltridecanoate; methyl 10,17-dimethyloctadecanoate; methyl 3-hydroxy-10,17-dimethyloctadecanoate; methyl 10,17-dimethylnonadecanoate; methyl 3-hydroxy-10,17-dimethylnonadecanoate; methyl 10,15-dimethylhexadecanoate; methyl 10,15-dimethylheptadecanoate; ethyl 10,17-dimethyloctadecanoate; ethyl 3-hydroxy-10,17-dimethyloctadecanoate; ethyl 10,17-dimethylnonadecanoate; ethyl 3-hydroxy-10,17-dimethylnonadecanoate; ethyl 10,15-dimethylhexadecanoate; ethyl 10,15-dimethylheptadecanoate; propyl 10,17-dimethyloctadecanoate; propyl 3-hydroxy-10,17-dimethyloctadecanoate; propyl 10,17-dimethylnonadecanoate; propyl 3-hydroxy-10,17-dimethylnonadecanoate; propyl 10,15-dimethylhexadecanoate; propyl 10,15-dimethylheptadecanoate; and combination(s) thereof.

28. The process of claim 25, wherein the fatty acid ester has one or more of the following properties:
a kinematic viscosity at 100° C. of less than 4.5 cSt;
a kinematic viscosity at 40° C. of less than 15 cSt;
a pour point of below −50° C.;
a Noack volatility of less than 14 wt %; and
a viscosity index of more than 120.

29. A lubricant comprising the fatty acid ester of claim 25.
